SEQUENCE LISTING (4 sequences)

SEQ ID NO: 1 - DNA, 111 bp, Artificial Sequence
  Description of Artificial Sequence Synthetic polynucleotide
atgtaataattataggcgatggagttcgccataaacgctgcttagctaatgactcctaccagtatcactactggtaggagtctatttttttgagcaagctatttaaagagg

SEQ ID NO: 2 - DNA, 159 bp, Bacillus subtilis
aaaaataatgttgtccttttaaataagatctgataaaatgtgaactaaatgtaataattataggcgatggagttcgccataaacgctgcttagctaatgactcctaccagtatcactactggtaggagtctatttttttgagcaagctatttaaagagg

SEQ ID NO: 3 - DNA, 10303 bp, Artificial Sequence
  Description of Artificial Sequence Synthetic polynucleotide
aattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctatcaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcccgacctcgagctggatacttcccgtccgccagggggacatgccggcgatgctgaaggtcgcgcgcattcccgatgaagaggccggttaccgcctgtttgaggatatagtaatctttctaaatagctttggattggaggagtatggccactaatactaagttcagctaataaaaaaatttgctaaagaactccagaaaagtaagcacctgttattgcaataaaattagcctaattgagagaagtttctatagaatttttcatatacttaacgagtgctttcacctttgaatatagtccttcccacttatcatcacactctccccgatagccttttctagctatatccagtaaagttacatgctctttaggtaaaagaggtatagcccattctgcagcgacatctttcgaggtaatttcaccagtagtcactgtttgccacattcgagctagggttaaaattacattacgctcatcaccttttatcccctcaattagttctggcaaagaatccttaattgctcttcgaatatctgtcaaaggtacggagacaagtatacttgaagaatcaggaccaaatagagaaatactattctttcttgcttgtgctaaaacaatagccaaatcaggatcatagcttggttcctgaatttgtccattctcaaattcacccctgagccactcaccgtatataaattctctttttggaggatattgccaagggacaacttcactcctatttataaccgtaacttcaagtggtctaacagaatccgtatttccaatctttcctgatatagtcattagtctttctgttagtttttttcgagttaattgaggtaaactatgattcacgacgactagaacatctacatcgctgttaatgcgtaaaccaccatttactgctgaaccaaatagatatactccaactattgaacttccaaataaatcttttacgatttttaatgtttgaatcgcttgatttggtatttttccgttaatcaaattgctcatgatttcacctcgttgattatgttcatataaagtttatattgatactcaatttacttaccctagattggacatatacttaaattactgttcaataaagctgaccgttagcgtttaagtacatcctttcacaatttgtctacagattaataattattctttattatacagatcgatcctctagacctaggccttaagatctgatcatatgcatccgcgggcccgggttaacgcgtaatccatggatcaagagacaggatgaggatcgtttcgcatgattgaacaagatggattgcacgcaggttctccggtgccctgaatgaactgcagaaagagctggtagttggcgcactgttcgaagaactgccgatgtccagtaagattcttactatgctggttgaaccggatgctggtaaagctacttgggttgctgcttctacttatggtaccgatacaactactggtgaggaagttaaaggagctcttaaagaaatccacttcagtaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgactaagaaaatgccgtcaaatccgctcgccatgacttcactaacgatgcctttgaaaatcttcaagttcttttctactaattcaaggcgtgtctcaccaggtttttggtttgctccggcgcaaatgcagacaatatcagcatccttgcagggtatgtttctctttgatgtctttttgtttgtgaagtatttcacatttatattgtgcaacacttcacaaacttttgcaagagaaaagttttgtctgatttatgaacaaaaaagaaaccatcattgatggtttctttcggtaagtcccgtctagccttgccctcaatggggaagagaaccgcttaagcccgagtcattatataaaccatttagcacgtaatcaaagccaggctgattctgaccgggcacttgggcgctgccattattaaaaatcacttttgcgttggttgtatccgtgtccgcaggcagcgtcagcgtgtaaattccgtctgcatttttagtcattggttttccaggccaagatccggtcaattcaattactcggctcccatcatgtttatagatataagcatttacctggctccaatgattcggattttgatagccgatggttttggccgacgctggatctcttttaacaaaactgtatttctcggtcctcgttacaccatcactgttcgttccttttaacatgatggtgtatgttttgccaaattggatctccttttccgattgtgaattgatctccatccttaaacgcctgtcgtctggtccattattgatttgataaacggcttttgttgtattcgcatctgcacgcaaggtaatcgtcagttgatcattgaaagaatgtgttacacctgttttgtaattctcaaggaaaacatgaggcgcttttgcaatatcatcaggataaagcacagctacagacctggcattgatcgtgcctgtcagtttaccatcgttcacttgaaatgaacccgctccagctttattgtcatacctgccatcaggcaattttgttgccgtattgatagagacagaggatgaacctgcatttgccagcacaacgccatgtgagccgcgctgattcataaatatctggttgtttccattcgggttcgagagttcctcaggctgtccagccatcacattgtgaaatctattgaccgcagtgatagcctgatcttcaaataaagcactcccgcgatcgcctatttggcttttccccgggaacctcacaccatttccgcctccctcaggtctggaaaagaaaagaggcgtactgcctgaacgagaagctatcaccgcccagcctaaacggatatcatcatcgctcatccatgtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgccgatgataagctgtcaaacatgagaattcaaaaataatgttgtccttttaaataagatctgataaaatgtgaactaaatgtaataattataggcgatggagttcgccataaacgctgcttagctaatgactcctaccagtatcactactggtaggagtctatttttttgagcaagctatttaaagaggggatccccagcttgttgatacactaatgcttttatatagggaaaaggtggtgaactactgtggaagttactgacgtaagattacgggtcgaccgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaatttcagctgagcgccggtcgctaccattaccagttggtctggtgtcaaaaataataataaccgggcaggccatgtctgcccgtatttcgcgtaaggaaatccattatgtactatcgatcagaccagtttttaatttgtgtgtttccatgtgtccagtttggaatactcttaacctcattggaaatcgcggcataatcactggtggtatgattgatgaccgcgtcaacaatgacctttatgccatattcttcagcggctgcacacatttctttaaattcttgttcagtacctaagtaacggttgccaatttgatacgatgtcggctgatacagccagtaccagttcgacatgcttttatctccttgattcccttcctttacttggttaatcggagatgtctgaatggctgtatatcctgcatcatgaatatccttcatattgtgttttaacgtattgaacgaccaattccatgcatgaagaatggttccgcttttgatcgacggtgctgtaagctcattcgatttgttcgccgtttcagcactcgcagccgccggtcctgccagaaccaaatgaaacagcaataaaaatccagcgaataacggcagtaaagaggttttgaatcgttttgcaaacattcttgacactccttatttgattttttgaagacttacttcggagtcaaaaatccctcttacttcattcttccgcttcctcctttcaaaccgatgtgaagactggagaattttgtt

SEQ ID NO: 4 - DNA, 12943 bp, Artificial Sequence
  Description of Artificial Sequence Synthetic polynucleotide
agtagttcaccaccttttccctatataaaagcattagtgtatcaacaagctggggatcctagaagcttatcgaattctcatgtttgacagcttatcatcggcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaaggtttttatattacagctccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagacgacaggcgtttaaggatggagatcaattcacaatcggaaaaggagatccaatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattgagggcaaggctagacgggacttaccgaaagaaaccatcaatgatggtttcttttttgttcataaatcagacaaaacttttctcttgcaaaagtttgtgaagtgttgcacaatataaatgtgaaatacttcacaaacaaaaagacatcaaagagaaacataccctgcaaggatgctgatattgtctgcatttgcgccggagcaaaccaaaaacctggtgagacacgccttgaattagtagaaaagaacttgaagattttcaaaggcatcgttagtgaagtcatggcgagcggatttgacggcattttcttagtcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctactgaagtggatttctttaagagctcctttaacttcctcaccagtagttgtatcggtaccataagtagaagcagcaacccaagtagctttaccagcatccggttcaaccagcatagtaagaatcttactggacatcggcagttcttcgaacagtgcgccaactaccagctctttctgcagttcattcagggcaccggagaacctgcgtgcaatccatcttgttcaatcatgcgaaacgatcctcatcctgtctcttgatccatggattacgcgttaacccgggcccgcggatgcatatgatcagatcttaaggcctaggtctagaggatcgatctgtataataaagaataattattaatctgtagacaaattgtgaaaggatgtacttaaacgctaacggtcagctttattgaacagtaatttaagtatatgtccaatctagggtaagtaaattgagtatcaatataaactttatatgaacataatcaacgaggtgaaatcatgagcaatttgattaacggaaaaataccaaatcaagcgattcaaacattaaaaatcgtaaaagatttatttggaagttcaatagttggagtatatctatttggttcagcagtaaatggtggtttacgcattaacagcgatgtagatgttctagtcgtcgtgaatcatagtttacctcaattaactcgaaaaaaactaacagaaagactaatgactatatcaggaaagattggaaatacggattctgttagaccacttgaagttacggttataaataggagtgaagttgtcccttggcaatatcctccaaaaagagaatttatatacggtgagtggctcaggggtgaatttgagaatggacaaattcaggaaccaagctatgatcctgatttggctattgttttagcacaagcaagaaagaatagtatttctctatttggtcctgattcttcaagtatacttgtctccgtacctttgacagatattcgaagagcaattaaggattctttgccagaactaattgaggggataaaaggtgatgagcgtaatgtaattttaaccctagctcgaatgtggcaaacagtgactactggtgaaattacctcgaaagatgtcgctgcagaatgggctatacctcttttacctaaagagcatgtaactttactggatatagctagaaaaggctatcggggagagtgtgatgataagtgggaaggactatattcaaaggtgaaagcactcgttaagtatatgaaaaattctatagaaacttctctcaattaggctaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatagtacataatggatttccttacgcgaaatacgggcagacatggcctgcccggttattaaaaaataatgttgtccttttaaataagatctgataaaatgtgaactaaatgtaataattataggcgatggagttcgccataaacgctgcttagctaatgactcctaccagtatcactactggtaggagtctatttttttgagcaagctatttaaagaggtactagtactgcagtccggcaaaaaagggcaaggtgtcctagtaaggtcgacaggaggactctctatgaaatttggaaactttttgcttacataccaacctccccaattttctcaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagtacgccaacttgaagatgtgaatttattggatcaaatgtcaaaaggacgatttcggtttggtatttgccgagggctttacaacaaggactttcgcgtattcggcacagatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagcttcgacgactgagtgggctgctcaatttggcctaccgatgatattaagttggattataaatactaacgaaaagaaagcacaacttgagctttataatgaagtggctcaagaatatgggcacgatattcataatatcgaccattgcttatcatatataacatctgtagatcatgactcaattaaagcgaaagagatttgccggaaatttctggggcattggtatgattcttatgtgaatgctacgactatttttgatgattcagaccaaacaagaggttatgatttcaataaagggcagtggcgtgactttgtattaaaaggacataaagatactaatcgccgtattgattacagttacgaaatcaatcccgtgggaacgccgcaggaatgtattgacataattcaaaaagacattgatgctacaggaatatcaaatatttgttgtggatttgaagctaatggaacagtagacgaaattattgcttccatgaagctcttccagtctgatgtcatgccatttcttaaagaaaaacaacgttcgctattatattagctaaggaggtaaagaaatgaaatttggattgttcttccttaacttcatcaattcaacaactgttcaagaacaaagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctgtcgccatagcggaggaagcttgcttattggatcagttaagtgaagggagatttattttagggtttagtgattgcgaaaaaaaagatgaaatgcatttttttaatcgcccggttgaatatcaacagcaactatttgaagagtgttatgaaatcattaacgatgctttaacaacaggctattgtaatccagataacgatttttatagcttccctaaaatatctgtaaatccccatgcttatacgccaggcggacctcggaaatatgtaacagcaaccagtcatcatattgttgagtgggcggccaaaaaaggtattcctctcatctttaagtgggatgattctaatgatgttagatatgaatatgctgaaagatataaagccgttgcggataaatatgacgttgacctatcagagatagaccatcagttaatgatattagttaactataacgaagatagtaataaagctaaacaagagacgcgtgcatttattagtgattatgttcttgaaatgcaccctaatgaaaatttcgaaaataaacttgaagaaataattgcagaaaacgctgtcggaaattatacggagtgtataactgcggctaagttggcaattgaaaagtgtggtgcgaaaagtgtattgctgtcctttgaaccaatgaatgatttgatgagccaaaaaaatgtaatcaatattgttgatgataatattaagaagtaccacatggaatatacctaataggtaccaggaggaaggcaaatatgactaaaaaaatttcattcattattaacggccaggttgaaatctttcccgaaagtgatgatttagtgcaatccattaattttggtgataatagtgtttacctgccaatattgaatgactctcatgtaaaaaacattattgattgtaatggaaataacgaattacggttgcataacattgtcaattttctctatacggtagggcaaagatggaaaaatgaagaatactcaagacgcaggacatacattcgtgacttaaaaaaatatatgggatattcagaagaaatggctaagctagaggccaattggatatctatgattttatgttctaaaggcggcctttatgatgttgtagaaaatgaacttggttctcgccatatcatggatgaatggctacctcaggatgaaagttatgttcgggcttttccgaaaggtaaatctgtacatctgttggcaggtaatgttccattatctgggatcatgtctatattacgcgcaattttaactaagaatcagtgtattataaaaacatcgtcaaccgatccttttaccgctaatgcattagcgttaagttttattgatgtagaccctaatcatccgataacgcgctctttatctgttatatattggccccaccaaggtgatacatcactcgcaaaagaaattatgcgacatgcggatgttattgtcgcttggggagggccagatgcgattaattgggcggtagagcatgcgccatcttatgctgatgtgattaaatttggttctaaaaagagtctttgcattatcgataatcctgttgatttgacgtccgcagcgacaggtgcggctcatgatgtttgtttttacgatcagcgagcttgtttttctgcccaaaacatatattacatgggaaatcattatgaggaatttaagttagcgttgatagaaaaacttaatctatatgcgcatatattaccgaatgccaaaaaagattttgatgaaaaggcggcctattctttagttcaaaaagaaagcttgtttgctggattaaaagtagaggtggatattcatcaacgttggatgattattgagtcaaatgcaggtgtggaatttaatcaaccacttggcagatgtgtgtaccttcatcacgtcgataatattgagcaaatattgccttatgttcaaaaaaataagacgcaaaccatatctatttttccttgggagtcatcatttaaatatcgagatgcgttagcattaaaaggtgcggaaaggattgtagaagcaggaatgaataacatatttcgagttggtggatctcatgacggaatgagaccgttgcaacgattagtgacatatatttctcatgaaaggccatctaactatacggctaaggatgttgcggttgaaatagaacagactcgattcctggaagaagataagttccttgtatttgtcccataatggaggtaaaagtatggaaaatgaatcaaaatataaaaccatcgaccacgttatttgtgttgaaggaaataaaaaaattcatgtttgggaaacgctgccagaagaaaacagcccaaagagaaagaatgccattattattgcgtctggttttgcccgcaggatggatcattttgctggtctggcggaatatttatcgcggaatggatttcatgtgatccgctatgattcgcttcaccacgttggattgagttcagggacaattgatgaatttacaatgtctataggaaagcagagcttgttagcagtggttgattggttaactacacgaaaaataaataacttcggtatgttggcttcaagcttatctgcgcggatagcttatgcaagcctatctgaaatcaatgcttcgtttttaatcaccgcagtcggtgttgttaacttaagatattctcttgaaagagctttagggtttgattatctcagtctacccattaatgaattgccggataatcttgattttgaaggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaaagaatcctgaggaggaaaacaggtatgacttcatatgttgataaacaagaaattacagcaagctcagaaattgatgatttgattttttcgagcgatccattagtgtggtcttacgacgagcaggaaaaaatcagaaagaaacttgtgcttgatgcatttcgtaatcattataaacattgtcgagaatatcgtcactactgtcaggcacacaaagtagatgacaatattacggaaattgatgacatacctgtattcccaacatcggtttttaagtttactcgcttattaacttctcaggaaaacgagattgaaagttggtttaccagtagcggcacgaatggtttaaaaagtcaggtggcgcgtgacagattaagtattgagagactcttaggctctgtgagttatggcatgaaatatgttggtagttggtttgatcatcaaatagaattagtcaatttgggaccagatagatttaatgctcataatatttggtttaaatatgttatgagtttggtggaattgttatatcctacgacatttaccgtaacagaagaacgaatagattttgttaaaacattgaatagtcttgaacgaataaaaaatcaagggaaagatctttgtcttattggttcgccatactttatttatttactctgccattatatgaaagataaaaaaatctcattttctggagataaaagcctttatatcataaccggaggcggctggaaaagttacgaaaaagaatctctgaaacgtgatgatttcaatcatcttttatttgatactttcaatctcagtgatattagtcagatccgagatatatttaatcaagttgaactcaacacttgtttctttgaggatgaaatgcagcgtaaacatgttccgccgtgggtatatgcgcgagcgcttgatcctgaaacgttgaaacctgtacctgatggaacgccggggttgatgagttatatggatgcgtcagcaaccagttatccagcatttattgttaccgatgatgtcgggataattagcagagaatatggtaagtatcccggcgtgctcgttgaaattttacgtcgcgtcaatacgaggacgcagaaagggtgtgctttaagcttaaccgaagcgtttgatagttga

(19) United States
(12) Patent Application Publication
(10) Pub. No.: US 2018/0265833 A1
(43) Pub. Date: Sep. 20, 2018

(54) MODULAR CHEMICAL SENSOR PLATFORM

(71) Applicants: John COPPETA, Windham, NH (US); John FUMO, Kailua Kona, HI (US); Amanda BILLING-SIUTI, Framingham, MA (US); Jason W. HOLDER, Swampscott, MA (US); THE CHARLES STARK DRAPER LABORATORY, INC., Cambridge, MA (US)

(72) Inventors: Jason W. Holder, Swampscott, MA (US); Robert A. Larsen, Somerville, MA (US); John Fumo, Kailua, Kona, HI (US); John Coppeta, Windham, NH (US); Ricky Soong, Rockville, MD (US); Amanda N. Billings-Siuti, Framingham, MA (US)

(21) Appl. No.: 15/756,784

(22) PCT Filed: Sep. 2, 2016
(86) PCT No.: PCT/US16/50131

§ 371 Date: Mar. 1, 2018

Related U.S. Application Data
(60) Provisional application No. 62/213,861, filed on Sep. 3, 2015.

(51) Int. Cl. C12M 1/34; C12M 1/02

(52) U.S. Cl. CPC C12M 41/32; C12M 27/00

(57) ABSTRACT

According to various aspects and embodiments, an integrated sensor system is provided. In one example, such an integrated sensor system includes at least one unit cell, an analysis device, and a controller. The at least one unit cell may include an activation chamber containing an activation fluid, a biological chamber containing a biological component, a first membrane disposed in between the activation chamber and the biological chamber, an assay chamber configured to receive a sample, and a second membrane disposed in between the assay chamber and the biological chamber.

### CROSS-REFERENCE TO RELATED APPLICATIONS

This application claims the benefit of priority under 35 U.S.C. § 119(e) to U.S. Provisional Application No. 62/213,861 filed on Sep. 3, 2015 and titled “MODULAR CHEMICAL SENSOR PLATFORM,” which is herein incorporated by reference in its entirety for all purposes.

### SEQUENCE LISTING

The instant application contains a Sequence Listing which has been filed electronically in ASCII format and is hereby incorporated by reference in its entirety. Said ASCII copy, created on Sep. 1, 2016, is named D2050-7046WO_SL.txt and is 31,459 bytes in size.

### FIELD OF TECHNOLOGY

One or more aspects of the disclosure relate generally to chemical sensors, and more particularly to engineered biological sensor systems and methods.

### SUMMARY

Aspects and embodiments are directed generally to integrated biological sensor systems and methods for tunable detection of specific analytes. According to some embodiments, a method for monitoring is provided. The method may comprise pressurizing a pressure chamber to apply a first pressure to an activation fluid contained in an activation chamber, pressurizing the activation fluid to apply a second pressure to a first membrane, the second pressure sufficient to rupture the first membrane and introduce the activation fluid to a biological chamber through the ruptured first membrane, combining in the biological chamber the activation fluid and a dried biological component contained in the biological chamber to form a reconstituted biological component, and pressurizing the biological chamber to apply a third pressure to a second membrane, the third pressure sufficient to rupture the second membrane and causing an entry of the reconstituted biological component to an assay chamber containing a sample such that the reconstituted biological component contacts the sample.

In some embodiments, the method further comprises determining a presence or an absence of at least one analyte of interest present in the sample using at least a portion of the reconstituted biological component that has contacted the sample, and sending a signal to a receiving device responsive to determining the presence or the absence of the at least one analyte of interest. According to another aspect, the method may further comprise measuring at least one optical characteristic of the portion of the reconstituted biological component that has contacted the sample. According to another aspect, the method may further comprise measuring at least one electrical characteristic of the portion of the reconstituted biological component that has contacted the sample.

In some embodiments, the optical characteristic is at least one of fluorescence, luminescence, and an absorption parameter. According to some aspects, determining the presence or the absence of the at least one analyte of interest is based on a fluorescence. According to another aspect, determining the presence or the absence of the at least one analyte of interest is based on an electric current output.

In some embodiments, the at least one analyte of interest includes fluoride and uranyl ions.

In some embodiments, the method further comprises receiving an activation signal to pressurize the pressurization chamber.

In some embodiments, pressurizing the pressure chamber comprises generating one or more gases within the pressure chamber. According to at least one embodiment, the pressure chamber comprises an aqueous solution, an anode, and a cathode, and generating the one or more gases comprises applying an electric current between the anode and the cathode so as to cause electrolysis of the aqueous solution. In some embodiments, the gas deforms an expandable membrane positioned between the pressure chamber and the activation chamber such that the activation fluid is pressurized by the deformed expandable membrane.

In some embodiments, the activation fluid is at least one of a culture media and a reaction media.

In some embodiments, the method further comprises freeze drying the biological component to generate the dried biological component, and pre-loading the biological chamber with the dried biological component.

In some embodiments, the dried biological component is at least one of an engineered biotic and an engineered abiotic. According to some embodiments, the engineered biotic is a genetically engineered microbe. According to some embodiments, the genetically engineered microbe is a bacterium. According to another embodiment, the engineered abiotic is an engineered DNA molecule. According to some embodiments, the engineered DNA molecule is configured to detect at least one metal ion.

In some embodiments, the reconstituted biological component comprises a molecule that has binding affinity for the analyte of interest.

In some embodiments, the method further comprises collecting the sample.

In accordance with one or more embodiments, an integrated sensor system is provided. The integrated sensor system may comprise at least one unit cell, the at least one unit cell including: an activation chamber containing an activation fluid, a biological chamber containing a dried biological component, a first membrane disposed in between the activation chamber and the biological chamber, an assay chamber configured to receive a sample, and a second membrane disposed in between the assay chamber and the biological chamber, an analysis device in communication with the assay chamber and configured to transmit an output signal based on detection of at least one characteristic of a biological component in contact with the sample, and a controller in communication with the analysis device and configured to receive the output signal.

In some embodiments, the first membrane is configured to be ruptured by a first predetermined pressure exerted by the activation fluid. In some embodiments, the second membrane is configured to be ruptured by a second predetermined pressure exerted by a reconstituted biological component.

In some embodiments, the integrated sensor system further comprises a pressure chamber, and an expandable membrane disposed between the pressure chamber and the activation chamber and configured to deform such that the expandable membrane applies pressure to the activation fluid.

In some embodiments, the pressure chamber further includes at least one electrode. According to another embodiment, the controller is configured to send an activation signal to the pressure chamber such that a voltage is applied the at least one electrode. In some embodiments, the pressure chamber contains a pressurization fluid. According to another embodiment, the pressurization fluid is an aqueous solution and an electric current is passed across the at least one electrode and passes through the aqueous solution to generate a gas. In some embodiments, the integrated sensor system further comprises a power supply coupled to the at least one electrode. In some embodiments, biological component comprising the dried biological component is configured to detect at least one analyte and to exhibit the at least one characteristic in the presence of the analyte.

In some embodiments, the unit cell further comprises a housing that at least partially encapsulates the activation chamber and the biological chamber. According to another embodiment, the housing is constructed from a light-transmissive material.

In some embodiments, the at least one characteristic is an optical characteristic. According to a further embodiment, the optical characteristic is at least one of fluorescence, luminescence, and an absorption parameter.

In some embodiments, the analysis device is a fluorimeter configured to measure fluorescence.

In some embodiments, the housing includes a patterned electrode configured to detect an electric current output by the portion of reconstituted biological component that contacted the sample. According to another embodiment, the analysis device is configured to measure the electric current.

In some embodiments, the analyte is a chemical presented as a vapor or an aerosol.

In some embodiments, the integrated sensor system comprises multiple unit cells.

In some embodiments, the reconstituted biological component is formed from at least a portion of the activation fluid entering through the first membrane and reconstituting at least a portion of the dried biological component contained in the biological chamber.

In some embodiments, the integrated sensor system is configured to operate in extreme conditions.

In some embodiments, the integrated sensor system is a modular platform and the at least one unit cell, the analysis device, and the controller function as modular elements.

In accordance with one or more embodiments, a method of facilitating an integrated sensor system for monitoring one or more target analytes is provided. The method may comprise providing an integrated sensor system, the integrated sensor system comprising at least one unit cell, the at least one unit cell including: an activation chamber containing an activation fluid, a biological chamber containing a dried biological component, a first membrane disposed in between the activation chamber and the biological chamber, an assay chamber configured to receive a sample, and a second membrane disposed in between the assay chamber and the biological chamber, an analysis device in communication with the assay chamber and configured to transmit an output signal based on detection of at least one characteristic of a biological component in contact with the sample, and a controller in communication with the analysis device and configured to receive the output signal, and providing instructions for activating the integrated sensors system.

In certain aspects, the present disclosure provides a nucleic acid (e.g., DNA or RNA) comprising: (a) a riboswitch (e.g., a fluoride-binding riboswitch, e.g., the riboswitch of SEQ ID NO: 1), (b) a reporter-encoding sequence operatively linked to the riboswitch such that the riboswitch controls activity, e.g., translation, of the reporter-encoding sequence, and (c) one or more (e.g., two) insertion sequences that direct insertion into a crcB locus in a genome. In embodiments, the insertion sequence is homologous (e.g., identical) to a corresponding crcB sequence in a target genome, e.g., in the genome of a bacterium capable of sporulation. In embodiments, the nucleic acid comprises DNA and a promoter directing expression of the riboswitch and reporter-encoding sequence.

In some aspects, the present disclosure provides a microorganism comprising: (a) a fluorine sensor, e.g., riboswitch, and (b) a crcB deficiency, e.g., deletion. In embodiments, the riboswitch is operatively linked to a reporter-encoding sequence such that the riboswitch controls activity, e.g., translation, of the reporter-encoding sequence. In embodiments, activity of the reporter-encoding sequence is more sensitive (e.g., by at least 2, 5, 10, 20, 50, 100, 200, 500, or 1000-fold) to fluorine than a reporter-encoding sequence in an otherwise similar microorganism having wild-type crcB.

Still other aspects, embodiments, and advantages of these example aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Embodiments disclosed herein may be combined with other embodiments, and references to “an embodiment,” “an example,” “some embodiments,” “some examples,” “an alternate embodiment,” “various embodiments,” “one embodiment,” “at least one embodiment,” “this and other embodiments,” “certain embodiments,” or the like are not necessarily mutually exclusive and are intended to indicate that a particular feature, structure, or characteristic described may be included in at least one embodiment. The appearances of such terms herein are not necessarily all referring to the same embodiment.

### DESCRIPTION OF THE DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1 illustrates a cross-sectional side view of one example of a unit cell in accordance with one or more aspects of the disclosure;

FIG. 2A illustrates a perspective view of one example of an integrated sensor system in accordance with one or more aspects of the disclosure;

FIG. 2B illustrates a cross-sectional side view of a portion of the integrated sensor system of FIG. 2A;

FIG. 3A and FIG. 3B are flow charts illustrating one example of a method according to one or more aspects of the disclosure;

FIG. 4A illustrates at least one act in the method of FIGS. 3A and 3B;

FIG. 4B illustrates at least one act in the method of FIGS. 3A and 3B;

FIG. 4C illustrates at least one act in the method of FIGS. 3A and 3B;

FIG. 4D illustrates an act in the method of FIGS. 3A and 3B;

FIG. 5 illustrates a schematic of an example of a process in accordance with one or more aspects of the disclosure;

FIG. 6 illustrates a schematic of another example of a sensor system in accordance with one or more aspects of the disclosure;

FIG. 7A is a graph illustrating a growth curve at a first temperature according to an experiment conducted in accordance with one or more aspects of the disclosure;

FIG. 7B is a graph illustrating a growth curve at a second temperature according to an experiment conducted in accordance with one or more aspects of the disclosure;

FIG. 7C is a graph illustrating a growth curve at a third temperature according to an experiment conducted in accordance with one or more aspects of the disclosure;

FIG. 7D is a graph illustrating a growth curve at a fourth temperature according to an experiment conducted in accordance with one or more aspects of the disclosure;

FIG. 8 is a chart illustrating the results of different culture mediums according to an experiment conducted in accordance with one or more aspects of the disclosure;

FIG. 9A is a first graph indicating arbitrary fluorescence units (AFU) at a first temperature according to an experiment conducted in accordance with one or more aspects of the disclosure;

FIG. 9B is a second graph plotting AFU at a second temperature according to an experiment conducted in accordance with one or more aspects of the disclosure;

FIG. 10A is a graph illustrating the results of luminescent reporting of fluoride in B. subtilis according to an experiment conducted in accordance with one or more aspects of the disclosure;

FIG. 10B includes photographs taken of engineered B. subtilis causing an increase in absorbance in response to different concentrations of fluoride according to an experiment conducted in accordance with one or more aspects of the disclosure; and

FIG. 10C includes photographs taken of engineered B. subtilis, with an additional deletion of the crcB gene, causing an increase in absorbance in response to different concentrations of fluoride according to an experiment conducted in accordance with one or more aspects of the disclosure.

### DETAILED DESCRIPTION

Traditional biological sensors are benchtop systems that address varied chemical and biological detection applications where sensor size and power consumption are largely unconstrained. Handheld or smaller scale sensing methods typically include solid-state devices and genetically-engineered biological sensors. More specifically, microelectromechanical systems (MEMS) that are based on functionalized microarrays, such as antibody coated SAW devices or micro-cantilever systems, exemplify solid-state devices. Genetically-engineered biosensors that employ bacteria as sensing elements can include whole-cell microsensors that can use fluorescent and bioluminescent cells coated on charge coupled device (CCD) chips.

Typical biological sensor systems and methods suffer from limited sensitivity and poor selectivity, which can lead to, for example, a high false positive rate. In addition, these systems are engineered to detect a narrow range of analytes, with detection of additional analytes only possible by re-engineering the entire sensor, and reconfiguring all the individual components. Further, the power consumption of typical biosensors is high, leading to a large package size to accommodate a battery to sustain weeks-long mission-life. Further, no genetically-engineered biosensor has yet been deployed in the environment as a persistent, unattended detector of target chemicals in the local, surrounding air.

The disclosure is directed to systems and methods of detecting analytes using an integrated sensor system, otherwise referred to herein as simply a “sensor system.” According to one or more aspects, the integrated sensor system may function as a biosensor. As used herein, the term “integrated,” when used in reference to the sensor system, refers to one or more components that may be incorporated into a single system and function cooperatively to achieve a specific result (e.g., to detect and generate a signal in response to one or more analytes). For example, according to at least one embodiment, and as discussed further below, the integrated sensor system includes a mechanical component, an electrical component, and a biological component. In some instances, one or more components may be incorporated into a single structure.

In accordance with some embodiments, the sensor systems may use one or more biological components that respond to target analytes, such as toxic substances, at a much lower concentration than humans can detect; thereby giving a warning of the presence of the toxic substances. For example, the sensor system may be used in any one or more different applications, including environmental monitoring, trace gas detection, water treatment facilities, production facilities, etc. In some embodiments, the sensor system may be used for security, such as homeland security, for the detection of narcotics, explosives, and other chemical substances. In other embodiments, the sensor system may be used in point-of-care diagnostics, for instance, for pathogen detection and epidemiological surveillance. In certain embodiments, the sensor system may be used to detect allergens, pollutants, agents of chemical and biological warfare, and agents that provoke a physiological response. The sensor systems disclosed herein may be suitable for a wide variety of applications, including ISR (Intelligence, Surveillance, and Reconnaissance) operations and systems.

According to some embodiments, the integrated sensor system may be placed in at least one of in-line, at-line, in-vivo, and in-vitro locations. For example, an in-line sensor system can be placed within a production line to monitor one or more variables associated with a continuous production line, and in certain instances can be automated, becoming another step in the process line. For instance, an in-line sensor system may be used in water purification processes. An at-line sensor system may be used in a production line where a sample can be taken, tested, and a decision can then be made as to whether production should continue to occur. A related example of an at-line sensor system is the monitoring of lactose in a dairy processing plant. An in-vivo sensor system may be one that is configured to function within the body, and in certain instances must be biocompatible and capable of interacting with the body during the intended period of the sensor system's use. In contrast, an in-vitro sensor system is intended to function outside the body, such as in a test tube, culture dish, or elsewhere outside of a living organism. The in-vitro sensor system may use a biological element, such as an enzyme, that is capable of recognizing or signaling a biochemical change in a solution. A transducer may then be used to convert the biochemical signal to a quantifiable signal. Other examples of in-vitro applications include gas, such as CO or CO₂, monitoring and detection.

According to various aspects, the sensor systems disclosed herein are capable of creating and/or maintaining a micro-environment for the biological component, such as microbes, thereby enabling long term storage in harsh environmental conditions, including arid deserts. The biological component can be activated on demand using an activation mechanism and used for detecting one or more target analytes. The biological component may comprise one or more engineered “reporter” probes that exhibit one or more optical or electrical characteristics in the presence of a target analyte. For instance, the biological component may fluoresce in the presence of a target analyte, such as a uranyl and/or fluoride ions. In some embodiments, a biological component may fluoresce different colors in the presence of different target analytes. For example, a bacterium may fluoresce at a first wavelength (e.g., green light) in the presence of a first target analyte, and the bacterium may fluoresce at a second wavelength (e.g., blue light) in the presence of a second target analyte. In other embodiments, one strain or type of bacteria may fluoresce one color in the presence of the first target analyte, and another strain or type of bacteria may fluoresce the same color or a different color in the presence of the second target analyte.

According to various aspects, the integrated sensor system may be an analytical device used for the detection of one or more analytes. In certain embodiments, the integrated sensor system includes a biological component, also referred to herein as a biological composition. The biological component may be a biologically sensitive element that interacts with a target analyte. The integrated sensor system may also include an analysis device for measuring one or more characteristics of a biological component that has interacted or otherwise come into contact with a sample and transmitting the results, and a controller configured to communicate with and control one or more other components of the system.

The integrated sensor systems and methods disclosed herein offer several advantages over typical biosensor systems. For example, the integrated sensor system may be a modular platform where one or more components, such as the biological component and the analysis device, function as modular elements. For example, the biological component and the analysis device may have one or more features or characteristics that can be changed or otherwise be reconfigured, but still function together within a larger unit. For instance, in some applications, the biological component may be configured to detect one or more different analytes. Likewise, the analysis device may be configured in one application to measure one or more characteristics, such as one or more optical characteristics, and in other applications, the analysis device may be configured to measure different characteristics, such as different optical characteristics or different characteristics altogether, such as electrical characteristics. According to another example, the biological component may include bacteria configured to sense more or different analytes, and/or may be configured to sense a lower or higher concentration of an analyte. The modular platform allows the overall system to offer considerable flexibility in arrangements so as to address specific criteria and constraints. However, some commonality is preserved so that each configuration of the sensor system does not have to be completely re-engineered.

The modular aspect of the sensor system allows for flexibility in constructing or otherwise configuring each of the components of the system. For example, the biological component may comprise one or more different elements that are configured to detect different target analytes. For instance, the biological component may comprise one or more biotic elements that are each configured to detect one or more target analytes. As used herein, the term “biotic” refers to a living component, such as a living organism, including bacteria and yeast. For instance, the biological component may comprise a type of bacteria possessing genetic elements that can sense a target analyte, e.g., one or more functional groups of a target analyte. In other embodiments, discrimination of target signatures can be accomplished through the cooperation of many different bacteria, each with a specialized sensing function, but can collectively coordinate with one another, such as through cell-cell communication, to perform sensing in a multiplexed way. According to another example, the biological component may comprise one or more abiotic elements that are each configured to detect one or more target analytes. As used herein, the term “abiotic” refers to a non-living component, such as a protein or nucleic acid, e.g., DNAzyme. According to a further example, the biological component may comprise both a biotic and an abiotic, where the biotic component is configured to detect one analyte and the abiotic component is configured to detect a different analyte than the biotic component.

According to some embodiments, the integrated sensor system may be configured to be remotely operated and may be suitable for use in external environments, including harsh environments with extreme conditions (deserts, coasts, high altitudes, extreme temperature conditions, sunlight, rain, wind, etc., or any environment away from the lab). As used herein, the term “extreme conditions” refers to an environment that includes any one or more conditions that include high temperature fluctuations, such as arid desert conditions where the diurnal temperature variation can be about 35° C., desert heat temperatures (e.g., 50° C.), high altitude, dust storms, etc. For instance, according to some embodiments, the integrated sensor system is configured to operate in an arid environment. According to some embodiments, the integrated sensor system is configured to operate under temperatures that fluctuate from about 4° C. to about 52° C. The integrated sensor system, including one or more components of the system, may be configured to operate for a desired application in any environment. In some instances, a biological component comprising B. subtilis may be used in applications where temperatures exceed 35 C. For example, depending on the amount of material loaded into the sensor system, B. subtilis may be capable of surviving at a temperature of 60° C. for over 26 days. In accordance with some embodiments, the disclosed sensor systems require low power to operate, and in certain instances may include the ability to be at least partially self-powered or entirely self-powered. Other advantages include the ability for the sensor system to be highly selective and/or highly sensitive, the ability to be small in size, and to have low manufacturing costs.

According to certain embodiments, the sensor system may be substantially self-powered, and in certain instances, may be entirely self-powered. For example, the sensor system may integrate a solar cell that functions to operate the sensor system. Output signals generated by the sensor system may need little or no additional power. For instance, according to some embodiments, the signals may be radio frequency (RF) and/or optical.

### Unit Cell

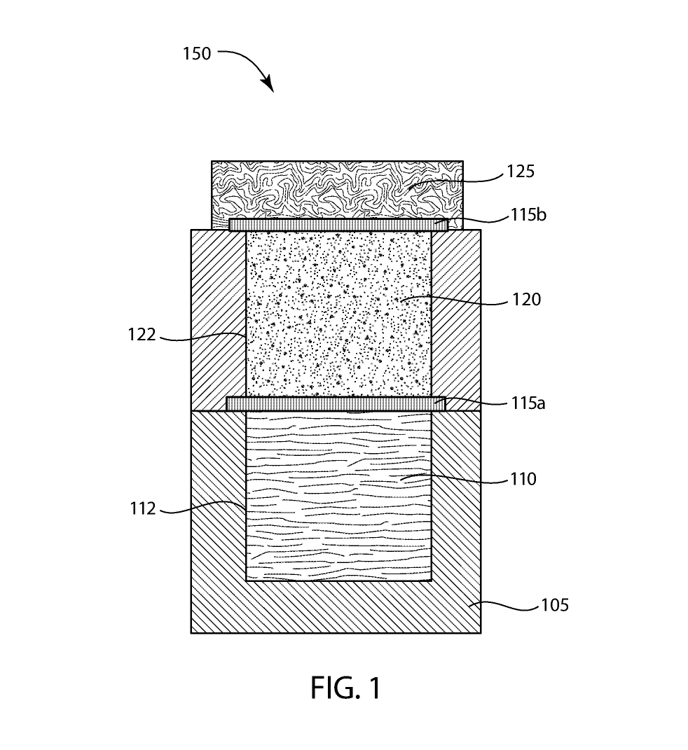

An exemplary embodiment of a unit cell 150 that may be used in an integrated sensor system is shown in FIG. 1. The unit cell 150 comprises an activation chamber 112, a biological chamber 122, an assay chamber 125, a first membrane 115a disposed between the activation chamber 112 and the biological chamber 122, and a second membrane 115b disposed between the biological chamber 122 and the assay chamber 125. Although the description below describes a unit cell 150 that includes an assay chamber 125, in some embodiments, the assay chamber 125 may not be included in or is otherwise separate from the unit cell 150. For example, an integrated sensor system may be configured such that multiple unit cells 150 are in fluid communication with or may otherwise service a single assay chamber 125. This allows for different biological components to be used for analyzing different samples and/or the same sample. Furthermore, according to some embodiments, the unit cell may include a pressure chamber, as discussed in further detail below. The pressure chamber may function to facilitate activation of activation fluid 110 disposed in the activation chamber 112.

The biological chamber 122 may comprise a biological component 120. In some embodiments, the biological component 120 may be a dried biological component. For instance, the biological component 120 may comprise freeze-dried cell based biological material, such as microbial cells, protozoal cells, animal cells, or plant cells. In some embodiments, the biological component 120 may comprise lyophilized bacterial cells or lyophilized nucleic acid enzymes. For instance, the dried biological component may be a DNA molecule, such as a DNAzyme.

According to various aspects, drying the biological component may extend the usable life of the biological component and allow the materials to be stored for prolonged periods of time. Drying the biological component may enable long term viability and functionality of both the biological component and the sensors. In some instances, drying the biological component allows the material to better withstand harsh environmental conditions, such as high temperatures, by minimizing undesirable molecular transformations caused by the external environment.

The activation chamber 112 may contain an activation fluid 110. The activation fluid 110 may be configured to reconstitute the dried biological component. For instance, the activation fluid 110 may reconstitute dried biological material, including bacteria, such that the bacteria are viable and retain cell integrity and cellular functionality after reconstitution. According to another example, the activation fluid may reconstitute DNA material such that it is capable of reacting in the presence of a target analyte. As used herein, the term “reconstitute” refers to the process of converting the dried biological component to a solution or suspension state or form through the addition of an activation fluid. Reconstitution refers to a process of dissolving or otherwise rehydrating dried biological components, such as lyophilized bacteria or DNAzymes, in a diluent such that the biological component is at least partially dispersed in the reconstituted biological component or composition. In a reconstituted biological component, especially a larger biological component such as a bacterium, some settling can occur.

In some embodiments, the activation fluid comprises a culture medium (or, in plural, culture media). According to some embodiments, the culture medium is an aqueous solution comprising one or more components for the dried biological component (such as bacteria), such that the biological component re-establishes metabolic activity and/or growth. In embodiments where the biological component comprises a spore, the culture medium can be medium suitable for germination, e.g., the medium comprises a germinant such as L-Alanine, e.g., at 1 mM.

According to some embodiments, the culture media comprises water. In some embodiments, the culture media comprises an isotonic solution. According to at least one embodiment, the culture media comprises agar. In some embodiments, the agar may be defined agar, and in other embodiments, the agar may be undefined agar, as understood by one skilled in the art. Selective growth compounds may also be added to the culture media. For example, the selective growth compounds may comprise antibiotics, carbon source(s), salts, and minerals. According to various aspects, an energy source may be included or otherwise incorporated with the culture media. The energy source may be selected based at least in part on the characteristics of the biological component. For example, in instances where the biological component is bacteria, the energy source may be selected based, at least in part, on whether the bacteria is heterotrophic or autotrophic.

In accordance with one or more embodiments, the culture media may contain one or more of a carbon and energy source, such as glucose, sucrose, tryptone, peptone, casein, or starch, an electron acceptor, such as nitrate, oxygen, fumarate, or pyruvate, a nitrogen source, such as ammonium salts, urea, yeast extract, casein, peptone, glutamate, glutamine, isoleucine, or other amino acids, an osmoprotectant (osmolyte), such as glycine betaine, or trehalose, a spore germination agent, such as L-alanine, L-valine, L-asparagine, glucose, fructose, potassium, calcium dipicolinic acid, or inosine, and/or one or more trace metals required for growth, such as zinc, magnesium, calcium, iron, manganese, boron, vanadium, cobalt, copper, selenium, molybdenum, or cobalt.

The culture media may be configured in such a way as to ensure that the biological component is viable for a certain period of time. For instance, the amount and the components used in the culture media may be proportioned to ensure that one or more organisms comprising the biological component material are viable for a length of time sufficient to detect a target analyte and exhibit one or more responsive characteristics.

According to some embodiments, the culture media or reaction media may include a hydrogel. The hydrogel may be a natural hydrogel or a synthetic hydrogel. According to other embodiments, the culture media or reaction media comprises a film. For instance, the culture media may comprise a biofilm, that includes electrospun polyvinyl alcohol, polyethylene oxide (PEO)-blend hydrogels, polyvinyl pyrrolidone, and Pluronic F127 dimethacrylate [FDMA or PEO₉₉-polypropylene oxide (PPO)₆₇-PEO₉₉ DMA]. In some embodiments, the FDMA may be crosslinked. In some embodiments, the biofilm may include FDMA/PEO-blend solutions. In some aspects, the FDMA/PEO-blend solution may have a weight ratio of 13:1. In other aspects, the FDMA/PEO-blend solution may have a weight ratio of 13:3.

According to some embodiments, the activation fluid 110 is a reaction medium. As used herein, the term “reaction medium” (or, in plural, “reaction media”) refers to a solution in which a reaction is performed. For instance, the reaction media may be a reaction buffer for a biological component 120, such as a DNAzyme. The reaction buffer may be an aqueous solution that is slightly acidic and may include one or more buffering agents and/or salts. For instance, according to some embodiments, a reaction medium may include one or more of a buffer to control pH (non-limiting examples including phosphate, bicarbonate, 3-(N-morpholino)propanesulfonic acid (MOPS), 4-(2-hydroxyethyl)-1-piperazineethanesulfonic acid (HEPES), etc.), and a salt that may function to provide suitable ionic strength (non-limiting examples including sodium chloride, potassium chloride or lithium chloride). According to various aspects, the reaction buffer allows the DNAzyme to become “activated” such that it is capable of detecting one or more target analytes. In some embodiments, the reaction media is an enzyme solution. For instance, the enzyme solution may comprise one or more cofactors for the enzyme (non-limiting examples including magnesium, calcium, thiamine, and pyridoxal phosphate), one or more additional substrates for the detection reaction (non-limiting examples including adenosine triphosphate, DNA, RNA, proteins, peptides, or a substrate for a colorimetric reaction), and a lyoprotectant, which functions to protect from possible damage during the lyophilization process.

In accordance with some embodiments, during operation of the sensor system the activation fluid 110 is pressurized with enough pressure such that the first membrane 115a is ruptured. The biological chamber 122 containing the inactive biological component may be positioned adjacent to the activation chamber 112 with the first membrane 115a disposed in between the activation chamber 112 and the biological chamber 122. The first membrane 115a may be in fluid communication with the activation fluid 112 and configured to obstruct flow of the activation fluid 110 until it is ruptured, whereby it functions as a passageway for the activation fluid 110 to enter the biological chamber 122. The biological chamber 122 may therefore be hermetically sealed until the first membrane 115a is ruptured.

In certain embodiments, the first membrane 115a may be constructed or otherwise formed from an inorganic material that ruptures at a predetermined pressure. Non-limiting examples of inorganic materials include, ceramics, glasses, or metals, or mixtures thereof. In some embodiments, the first membrane 115a material comprises silicon (Si). For instances, in some embodiments, the first membrane 115a material is silicon nitride (SiN). Other non-limiting examples of materials that may be used as the first membrane 115a include silicon oxide (SiO₂), aluminum oxide (Al₂O₃), or tetraethyl orthosilicate (TEOS). According to other embodiments, the first membrane 115a may be constructed from an organic material, such as a polymer material that ruptures at a predetermined pressure. The membrane material may also be inert to both the activation fluid 112 and the biological component 120.

The first membrane 115a may have a thickness that is configured to rupture when pressure within the activation chamber exceeds a predetermined level. The thickness of the first membrane 115a may depend on the membrane material, the size and shape of the membrane, and the predetermined rupture pressure. The first membrane 115a may therefore be nanometers to millimeters in thickness. In instances where pressure is generated using electrolysis (discussed further below), the pressures may exceed 100 atm, and may even exceed several hundred atmospheres. The range of pressures at which the first membrane 115a may be configured to rupture at may therefore be any one of these pressures. In some embodiments, the first membrane 115a may be configured to rupture at pressures that range from 1 psi to 300 atm. According to one embodiment, the first membrane 115a may have a thickness of about 50 nm and rupture at a pressure of about 10 psi.

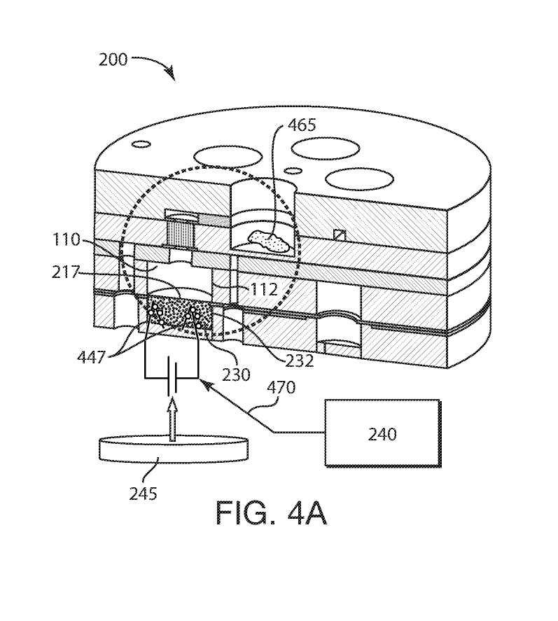

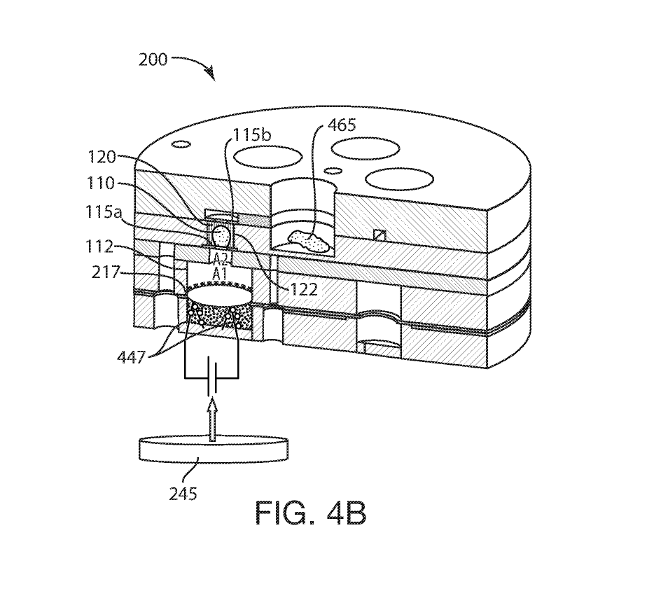

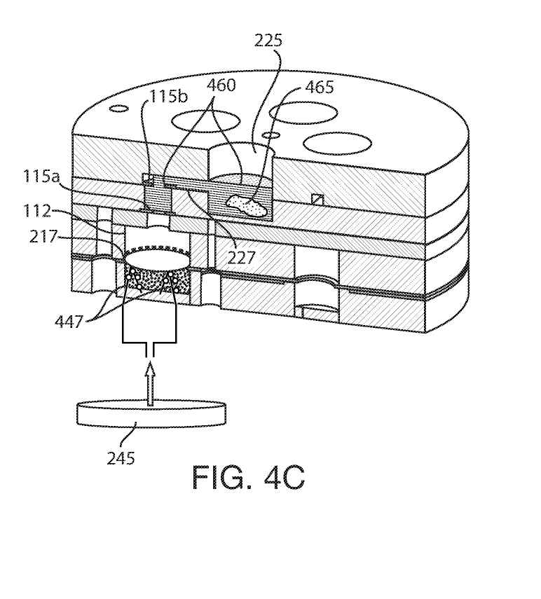

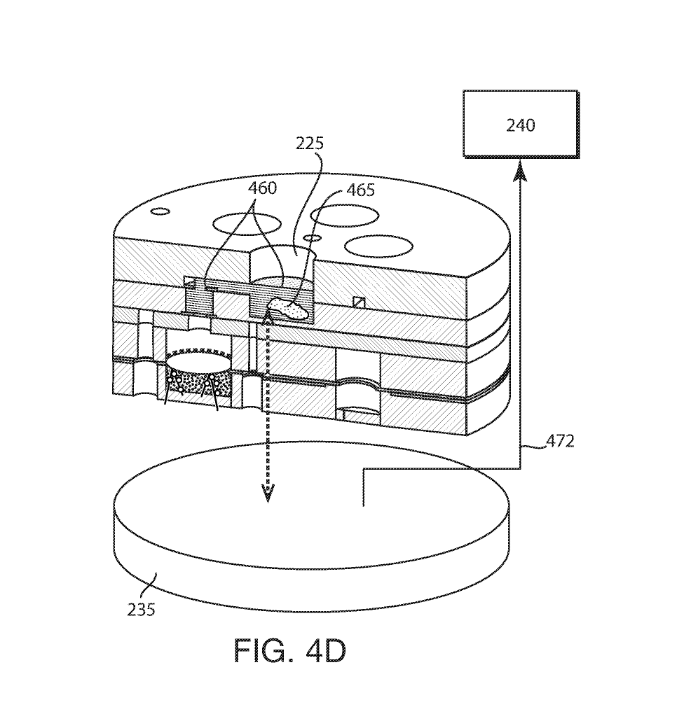

Once the activation fluid 110 has entered the biological chamber 122, it combines with the biological component 120 disposed in the biological chamber 122 to form a reconstituted biological component (i.e., see 260 in FIG. 4C). The reconstituted biological component may be pressurized so as to rupture the second membrane 115b. According to some embodiments, the second membrane 115b may be configured in substantially the same way as the first membrane 115a. For instance, the second membrane 115b may be constructed from the same materials as the first membrane 115a and configured such that the second membrane 115b ruptures at the same predetermined pressure as the first membrane 115a. In other embodiments, the second membrane 115b may be constructed from different polymer materials and/or thicknesses such that the second membrane 115b ruptures at a different pressure than the first membrane 115a.

The assay chamber 125, also referred to herein as a sample trap or sample chamber, is configured to receive a sample from the external environment. For instance, the unit cell 150 may be integrated into a larger sensor system (discussed in further detail below) and placed in the vicinity of an area of interest. The area of interest may be, for example, a facility or site suspected of manufacturing or assembling weapons of mass destruction (WMD). Airborne molecules produced by such a site may enter the assay chamber 125 as a sample, where they come into contact with reconstituted biological component that is configured to detect one or more target analytes that may be present in the sample. The assay chamber 125 may be configured as a permeable enclosure that allows ingress of a sample, such as an airborne molecule. The assay chamber 125 may also be constructed to retain reconstituted biological component that enters from the biological chamber 122 through the ruptured second membrane 115b. In this example, the assay chamber 125 is positioned adjacent to the biological chamber 122 such that the assay chamber 125, the second membrane 115b, and the biological chamber 122 (and the activation chamber 112) lie along a common axis. In other embodiments discussed below, the assay chamber 125 may not be on a common axis with the biological chamber 122 or the activation chamber 112. The assay chamber 125 may be in fluid communication with the biological chamber 122 via one or more channels or fluid flowpaths.

In accordance with at least one embodiment, the unit cell 150 may also comprise a housing 105 that is configured to at least partially encapsulate the unit cell 150. In some instances, the housing 105 at least partially encapsulates the activation chamber 112 and the biological chamber 122. According to some embodiments, the housing may be constructed from a light-transmissive material, such as a transparent polymer. In one embodiment, the light-transmissive material facilitates the detection and measurement of optical characteristics associated with reconstituted biological component that contacts the sample, as discussed in further detail below. For instance, the light-transmissive material may facilitate measurements associated with at least one of fluorescence, luminescence, and an absorption parameter of the reconstituted biological sample.

According to some embodiments, the housing may include at least one electrode, such as a patterned electrode. In certain embodiments, the electrode may be configured to detect an electric current output by reconstituted biological component that contacts the sample, as discussed in further detail below.

The housing 105 may also be configured so as to hermetically seal the biological chamber 122 and/or the activation chamber 112. This may allow the sensor system to be deployed and left for long periods of time in harsh environmental conditions and yet still maintain the viability and functionality of the biological component 120 and the activation fluid 110.

In accordance with some embodiments, the housing may also incorporate or include one or more other functions. For instance, the housing 105 may comprise one or more electronic components configured to receive and transmit signals from a controller. According to another example, the housing 105 may further comprise a source of power, such as a battery. According to one example, the housing 105 may include a battery-powered solid-state readout circuit. In some instances the housing 105 may comprise a computer chip that functions as a processor.

### Integrated Sensor System

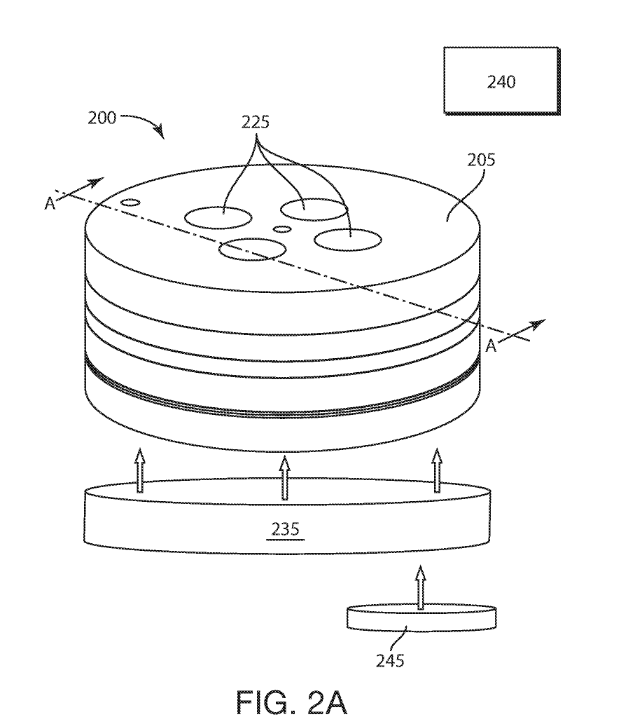

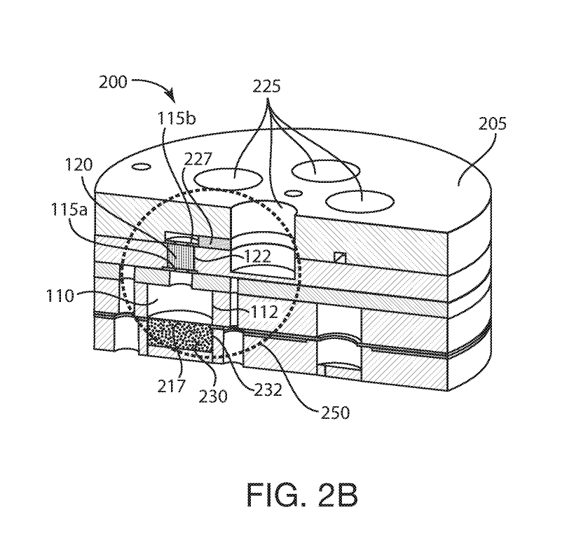

In accordance with at least one embodiment, an example of an integrated sensor system 200 is shown in FIGS. 2A and 2B. The sensor system of FIGS. 2A and 2B includes one or more unit cells 250, an analysis device 235, a controller 240, and a power source 245. FIG. 2B is a cross-section of a portion of the integrated sensor system 200 of FIG. 2A and includes a cross-sectional view of one example unit cell 250 within a housing 205 that may be included in the integrated sensor system 200. For purposes of convenience, the analysis device, 235, the controller, 240, and the power source 245 are not shown in FIG. 2B.

The sensor system 200 may also include a housing 205 that encloses the one or more unit cells 250. The housing 205 may be constructed and provide the functionalities as described above in reference to housing 105 of FIG. 1. The housing 205 may be constructed from a material that is capable of providing a hermetic environment to at least one of the pressure chamber, activation chamber 112, and biological chamber 122. The housing 205 may be a polymer material, non-limiting examples including polycarbonate, polypropylene, polystyrene, polymethyl methacrylate, polyethylene terephthalate, etc. According to other embodiments, the housing 205 may be constructed from one or more of a glass, a ceramic, or a metal. For instance, the housing 205 may be constructed from a metal material such as steel or aluminum. Electric components such as electrodes and wiring may be insulated from the housing. The housing 205 may also be constructed from materials capable of withstanding extreme conditions, as described above. As discussed in more detail below, the housing 205 may also include at least a portion of the analysis device 235, the power source 245, and in certain instances, the controller 240.

As shown in FIG. 2B, the unit cell 250 includes an activation chamber 112 comprising activation fluid 110, a biological chamber 122 comprising a biological component 120, a first membrane 115a, and a second membrane 115b that are each provided as described above in reference to FIG. 1. In the example shown in FIG. 2B, the unit cell 250 also includes a pressure chamber 232 and an expandable membrane 217 disposed in between the activation chamber 112 and the pressure chamber 232. According to at least one embodiment, and as discussed below, the pressure chamber 232 may be configured as an enclosed space and may be configured to apply pressure to the activation fluid 110 in the activation chamber 112. For instance, the pressure chamber 232 may be pressurized so as to deform the expandable membrane 217 and apply pressure to the activation fluid 110.

According to some embodiments, the expandable membrane 217 may be constructed from a polymer material that is configured to withstand pressure exerted by the pressurization fluid 230. This pressure may be sufficient to allow the expandable membrane 217 to pressurize the activation fluid 110 such that the first membrane 115a ruptures. The expandable membrane 217 is configured to expand and not rupture and therefore may be constructed from a material that has a high modulus of elasticity. The expandable membrane 217 may be constructed from an elastomeric material that is configured to expand. For instance, the expandable membrane 217 may be an elastomeric polymer material. Non-limiting examples of such materials include polyurethane, ethylene vinyl acetate, polyethylene, polyester, and flexible polyolefins. In some embodiments, the expandable membrane 217 may be a metal material. For instance, the metal material may be constructed from an expandable metal material and include one or more corrugations to direct the direction of expansion. The metal material may be any metal material capable of being configured to function as the expandable membrane, such as metal alloys, aluminum, steel, etc. In instances where polymer material is used for the first and second membranes 115a and 115b, the expandable membrane 217 may be configured to be thicker and constructed from materials that are stronger than the first and second membranes 115a and 115b. The thickness of the expandable membrane 217 may depend on the application, the size and shape of the membrane, the desired pressures for operating the sensor, and the material used to form the membrane. In some embodiments, the expandable membrane 217 may have a thickness of less than 10 mils, but it is to be appreciated that thicker expandable membranes are also within the scope of this disclosure. According to one embodiment, the expandable membrane has a thickness of about 1 mil.

The integrated sensor system 200 also includes an assay chamber 225. The assay chamber 225 may be configured to receive a sample and may be constructed so as to provide similar functionality as the assay chamber 125 discussed above in reference to FIG. 1. In this instance, the assay chamber 225 is separated from the second membrane 115b and the biological chamber 122 by a channel 227 that functions to deliver the reconstituted biological component to the assay chamber 225. Thus, the biological chamber 122, the second membrane 115b, and the assay chamber 225 are not on a common axis, in contrast to the configuration shown in FIG. 1.

In accordance with various aspects, one or more unit cells may be included in the integrated sensor system. For instance, FIGS. 2A and 2B include four unit cells, although it is to be appreciated that other configurations are within the scope of this disclosure. For example, the sensor system may include one unit cell, or hundreds of unit cells. The number of unit cells may depend on the application, the size of the unit cell, and the desired size of the device, and therefore allows for an infinite number of arrangements. The inclusion of multiple unit cells allows for sequential, separate, activation of each unit cell included in the structure over a period of time. The biological component included in each unit cell is preserved until activated by the activation fluid. Each unit cell can therefore be activated at sequentially different times, which extends the working life of the device. In some embodiments, the unit cells may be activated such that their respective detection periods overlap. This may be useful in applications where continuous monitoring is desired. In other embodiments, the unit cells may be activated such that the detection periods are discrete and separate from one another. This type of monitoring scheme may apply in instances where isolated periods of monitoring are desired, e.g., after a suspected release of a target analyte.

Referring back to FIG. 2A, the integrated sensor system 200 may also include a power source 245. In some embodiments, the power source 245 may be a low power supply. For instance, in some embodiments, the power source 245 may be a “button cell” or “coin cell” as understood by those skilled in the art. In some embodiments, the power source 245 may be configured to supply power as needed for a predetermined length of time, such as several weeks, to one or more components of the integrated sensor system 200. In some embodiments, one or more components of the sensor system may require less than 1 mW of power. The power source 245 may be incorporated into the housing 205 of the integrated sensor system 200. For instance, the power source 245 may be positioned on a bottom surface of the housing 205, such that it can be removed and replaced. In some embodiments, the power source 245 is incorporated into a surface or body of the analysis device 235. In this instance, the power source 245 may provide power to other components of the integrated sensor system 200, i.e., to apply a voltage to circuitry associated with the pressure chamber, as well as providing power to the analysis device 235.

As shown in FIG. 2B, the analysis device 235 may be coupled to the integrated sensor system 200 and may be in communication with the assay chamber 225. The analysis device 235 is configured to detect and/or measure at least one characteristic of the biological component that contacts a sample present in the assay chamber 225. The at least one characteristic may indicate the presence and/or quantity of the target analyte of interest.

Depending on the characteristic being examined, the analysis device 235 may be positioned and configured in different ways in relation to the unit cell. FIG. 2A indicates one embodiment where at least a portion of the analysis device 235 may be attached to a bottom surface of the integrated sensor system 200. In other embodiments, the analysis device 235 may be positioned on a top or bottom surface of the system 200, or may be integrated into the housing 205 itself. The analysis device 235 may be positioned anywhere within the integrated sensor system 200 suitable for purposes of performing the analysis functions as described in the methods and systems disclosed herein. The analysis device 235 may also comprise separate components that are positioned within the integrated sensor system 200. For instance, one or more sensors for detecting characteristics of the biological component may be positioned within the assay chamber and electrical or network circuitry may be coupled to the sensors to power and/or transmit data to and from the sensors. A processor may also be included in the analysis device 235 that is coupled to the sensors and electrical or network circuitry. In accordance with some embodiments, the analysis device or housing 205 of the sensor system may include a battery-powered solid-state readout circuit.

The analysis device 235 may be configured to transmit an output signal based on the detection and/or measurement conducted on the biological component. In some embodiments, the output signal may convey information such as a positive detection of one or more target analytes. In some embodiments, the output signal may convey information such as the amount or quantity of target analyte detected. In some instances, the integrated sensor system may be configured to detect and/or measure more than one target analyte.

In accordance with some embodiments, the analysis device 235 may be configured to detect and/or and measure at least one optical characteristic of biological component that contacts the sample. As used herein, the term “optical characteristic” refers to how the biological component affects the spectral properties of electromagnetic radiation, such as the absorbance, fluorescence, phosphorescence, and emission of electromagnetic radiation, including ultraviolet, visible, and/or infrared radiation. For instance, the optical characteristic may be at least one of fluorescence, luminescence, and an absorption parameter.

According to one embodiment, the analysis device 235 may be configured as or otherwise function as a fluorometer for conducting fluorometric analysis of the biological component. Fluorescence may be detected and measured using an emission based absorbance measurement. The fluorometer measures fluorescence by supplying an excitation source, detecting the resulting emission, and then converting the emission into an electrical signal proportional to fluorescence. The fluorescent signal is proportional to analyte concentration. According to some embodiments, the fluorometer may incorporate a light source, such as an LED light source, and an optical spectrum analyzer that functions to measure optical power as a function of wavelength. Incoming light passes through an optical filter, e.g., a wavelength-tunable optical filter that functions to select the optimal emission wavelength, which is different from the excitation wavelength. A photodetector may then convert the optical signal to an electrical current proportional to the incident optical power. In some embodiments, the photodetector comprises a photodiode. This electrical current may then converted to a voltage by an amplifier and digitized. An output signal signifying the detection and strength of the fluorometric signal may then be output by the analysis device 235 to the controller 240, for example, through a wireless transmission link. In some embodiments, the analysis device 235 includes a separate controller. For instance, a fluorometer may include a microcontroller that functions to activate, measure, and send a signal to the controller 240.

In accordance with some embodiments, one or more fluorometers may be configured to be integrated within the sensor device. For instance, a sensor device having a size less than 5 cm³ may include several assay chambers that each includes their own fluorometer device. In some embodiments, the fluorometer may be a separate modular unit that is integrated into the sensor as substantially one piece. In other embodiments, one or more components of the fluorometer may be integrated into the sensor device separately but still communicate with one another. For instance, the light source may be located in one location such as a sidewall of the assay chamber or elsewhere in the housing, and the controller may be located in another location in the sensor device and connected to the light source via circuitry and/or data connectors, such as USB connectors.

According to some embodiments, the analysis device may be configured to detect and measure light emitted from the biological sample. The luminescent light emitted from the biological sample may be measured by the analysis device, and generate an output signal representative of the amount of emitted light. For instance, the analysis device may function as a photometer or a photodetector that is configured to collect and measure light (e.g., lumens) emitted from the biological sample to determine the type and/or amount of target analyte is in the sample. For example, the presence of light emitted by the biological component may indicate the detection of a target analyte, and the intensity of the emitted light emitted may be correlated with the amount or concentration of target analyte in the sample.

According to one or more embodiments, a reaction of the biological component with a target analyte will affect the absorption properties of the biological component. For instance, the reaction may cause the biological component to emit light at one or more characteristic visible wavelengths of light. In some embodiments, the analysis device may be configured to conduct colorimetric analysis of the biological component for purposes of detecting one or more target analytes. In some embodiments, the analysis device may be a photometer. In some instances, the analysis device may be a spectrophotometer and measure both absorption properties (i.e. wavelength), as well as the amount of light (e.g., luminescence, or the intensity at the emitted wavelength) emitted from the biological component.

According to some embodiments, the analysis device may be configured to detect and measure one or more of fluorescence, luminescence, and absorption properties. For instance, one or more sensors of the analysis device may be configured to detect and measure the amount of light and the wavelength of light output by the biological sample. Depending on the type of photons emitted from the biological sample, the analysis device may conduct a fluorometric analysis. According to some embodiments, the light emitted by the biological composition may be measured with and without excitation to infer fluorescence. The analysis device may therefore also include one or more sources of light that function as an excitation light source. In some embodiments, the excitation light source may be a single wavelength excitation light source. In some embodiments, the analysis device may be configured with one or more emission filters to filter the emitted light and transmit one or more wavelengths of light. In embodiments the light is transmitted to a detector that then measures one or more properties of the transmitted light. In some instances, the analysis device may include both excitation light source(s) and emission filters.

According to some embodiments, the analysis device is in fluid communication with the assay chamber. For instance, a channel (not shown) may provide fluid communication between the assay chamber and one or more components of the analysis device such that a liquid sample containing the biological component passes through the channel to the analysis device to be analyzed. According to some embodiments, the sample may be fluidly coupled to the analysis device through the process of diffusion. In accordance with certain embodiments, a microfluidic channel may allow the sample to be fluidly coupled to the analysis device. The flow of the sample may be achieved by capillary action and/or diffusion, which may eliminate the need for pumps or other fluid flow devices. In other embodiments, the assay chamber may be integrated with the analysis device. For instance, one or more sensors associated with the analysis device may be disposed on the walls of the assay chamber.

In accordance with some embodiments, the analysis device may be configured to detect and/or measure an electric current output by a biological component that contacts the sample. In some embodiments, electric current emitted by the biological component may indicate the presence of one or more target analytes, and the amount of current may be correlated with the concentration or amount of target analyte that is present in the sample. For example, the analysis device may comprise a multimeter configured to measure the electric output of the biological component.

To implement a measurement for electric current, the integrated sensor system may include at least one electrode that is in communication with the analysis device. According to some embodiments, the housing of the integrated sensor system may include at least one electrode. For example, at least one electrode may be positioned or otherwise located in the vicinity of the assay chamber, such as a bottom surface of the assay chamber. The at least one electrode may include patterned electrodes or traces. For instance, one or more surfaces of the assay chamber may include a patterned electrode. Biological component that emits electric current in the presence of one or more target analytes may contact the at least one electrode and thereby complete an electric circuit, which can be detected and measured by the analysis device.

According to some embodiments, the analysis device may be configured to detect and/or measure both optical and electrical characteristics. In accordance with at least one embodiment, the analysis device may be equipped with one or more sensors that measure spectral properties of the biological component, as well as electrodes to measure electrical properties. In some embodiments, the integrated sensor system, such as the housing, may be possess both light transmissive properties and include a patterned electrode. For instance, sidewalls of the assay chamber may be constructed from light-transmissive material and the bottom surface of the assay chamber 125 may include a patterned electrode. In other embodiments, a channel may siphon off a portion of the biological component that has contacted the sample and transfer this portion to a chamber located in the analysis device. This chamber may be configured to measure at least one optical and/or electrical properties of the biological component.

According to some embodiments, the analysis device may be characterized as a low power device. For instance, the analysis device may require less than about 1 mW to operate. In accordance with some embodiments, the analysis device may be coupled to and powered by power source 245. In other embodiments, the analysis device may incorporate a separate source of power. For instance, a low power device, such as a “coin cell” may be integrated with or otherwise coupled to the analysis device to provide power to the device.

In some embodiments, the analysis device may be constructed from a light-transmissive material, as described above. The analysis device may also be small in size. For instance, in some embodiments, the analysis device may be less than 5 in³ in size.

The controller 240, also referred to herein as a receiving device, is configured to receive the output signal transmitted by the analysis device 235. The controller may be a computer, as known in the art. According to certain embodiments, the controller 240 may be positioned externally or remotely from the housing 205. In some embodiments, portions of the controller 240 may be positioned in more than one location remotely from the housing 205. For example, the housing may include the unit cells, the analysis device, and the power source, and may be deposited in one location where monitoring of one or more target analytes is desired, such as in a desert region. One component or portion of the controller may remotely send an activation signal to the housing and another component or portion of the controller may receive the transmitted output signal from the analysis device in the housing. For instance, a controller positioned at a first remote location, such as a ground-based control center may send an activation signal, and a controller positioned at a second remote location, such as an airplane, may receive the transmitted output signal.

The activation and output signals may be transported wirelessly through radio frequency signals to and from the controller. For instance, the analysis device may comprise, for example, a radio frequency identification (RFID) tag and the controller may comprise a reader for receiving and processing signals from the RFID tag. The reader may be configured to measure a signal from the RFID tag and at least one additional parameter from which a signal is derived. The RFID tag may be passive, semi-passive, or active. In some embodiments, the controller may comprise one or more amplifiers, filters, and multiplexers. According to some embodiments, the controller may comprise one or more analog-to-digital converters, linearizers, and compressors.

Referring back to FIG. 2B, in this example, each unit cell 250 is associated with a single assay chamber 225. According to other embodiments, multiple unit cells may be associated with a single assay chamber. For instance, in some embodiments, multiple unit cells may be activated to introduce separate reconstituted biological components into the assay chamber. This may be useful in applications where one biological component is configured to detect one target analyte in the sample, and another biological component is configured to detect a different target analyte in the sample. Multiple biological chambers may also be useful when the respective biological components are not compatible with one another or require different activation fluids to become reconstituted.

The integrated sensor systems disclosed herein may be configured to be of any size or shape suitable for a particular application. In certain instances, the sensor system may be less than 5 cm³ in size. According to some embodiments, the sensor system may be less than 2.5 cm³ in size. In some embodiments, the sensor system may be approximately 1 cm³ in size. According to other examples, the sensor system may be micron-sized or smaller. The small size of the sensor system may make it easier to conceal and to leave during longer periods of deployment.

In accordance with certain aspects, the integrated sensor system is configured as a modular platform. For instance, at least one of the unit cell, the analysis device, and the controller may function as modular elements. The housing may be configured to accept any one of a number of different, interchangeable unit cells or different, interchangeable analysis devices. For example, an application that calls for one particular type of target analyte may require a unit cell equipped with a first type of biological component and a first type of analysis device. These elements can be inserted into the housing and the system can then be deployed. A different application may require a different unit cell and a different analysis device. In some instances, the housing can be retrieved and new, unused unit cells can be included in the housing for a separate deployment. The analysis device may be swapped out or used over and over again. Furthermore, one application may require an internal controller located within the housing, whereas another application requires an external controller. In some embodiments, the components of the unit cell can be swapped out. For instance, a different bacterial composition and activation fluid may be implemented for a particular application by simply replacing the biological chamber and activation chamber. In some instances, the pressure chamber may also be swapped out. For example, different biological components or activation fluids may require a different amount of pressure applied to the first and second membranes. As will be appreciated, many different configurations can be executed based on a specific application.

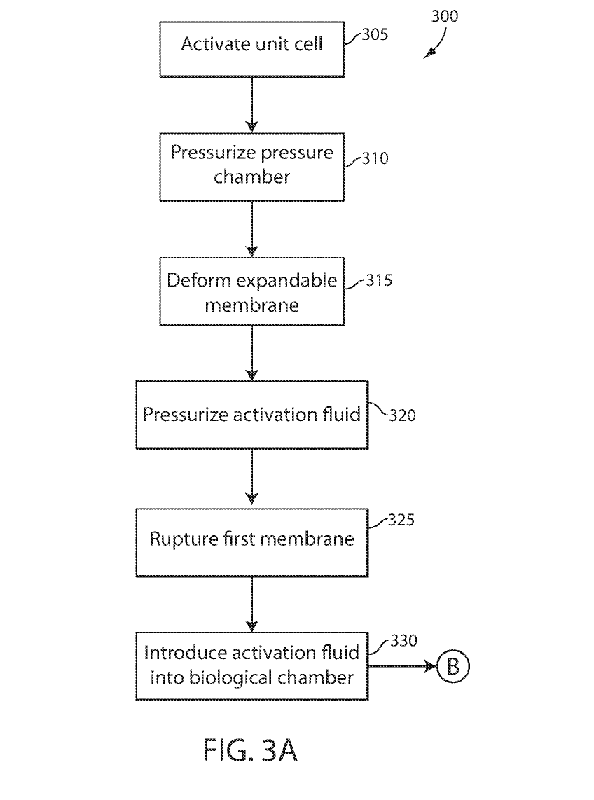

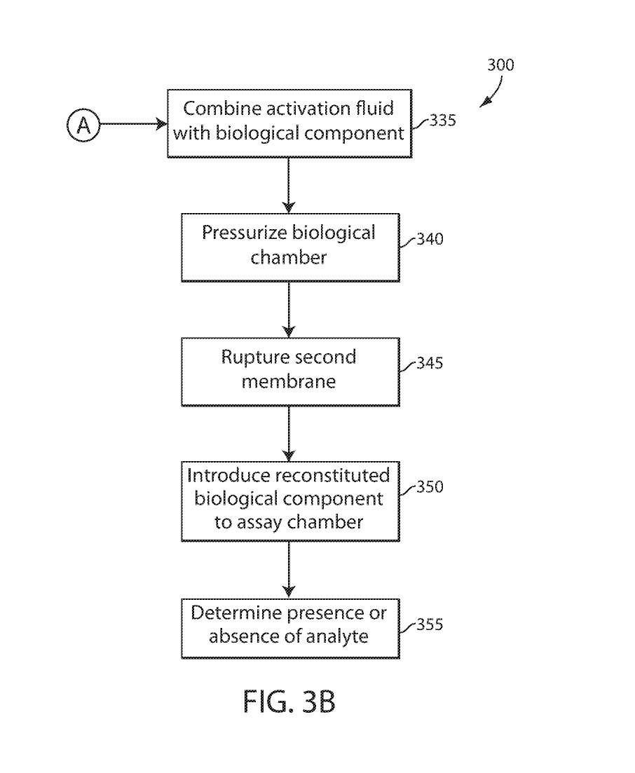

FIGS. 3A and 3B illustrate, in a flow diagram, one example of a method 300 for monitoring in accordance with one or more aspects of the disclosure. Method 300 is described below in reference to FIGS. 4A-4D, which shows operation of the integration sensor system of FIGS. 2A and 2B.

A first act 305 includes activating a unit cell of the integrated sensor system. As used herein, the term “activate” refers to an associated control command that is generated to prompt one or more components of the integrated sensor system to activate an associated functionality. FIG. 4A illustrates one example of activation in accordance with one embodiment. For purposes of convenience, the analysis device 235 is not shown in FIG. 4A.

According to one embodiment, the pressure chamber 232 includes at least one electrode 447 and the power source 245 may be coupled to the at least one electrode 447. For instance, the pressure chamber 232 may include at least one of an anode and a cathode. The controller 240 may send an activation signal 470 to the integrated sensor system such that a voltage is applied to the electrode 447 from the power source 245 and an electric current is passed across the electrode and passes through the pressurization fluid 230. In some embodiments, the pressurization fluid 230 is an aqueous solution, such as water or an alkaline water solution such as sodium hydroxide or potassium hydroxide. The electric current that passes through the aqueous solution may generate one or more gases, including oxygen and hydrogen. The one or more generated gases pressurize the pressurization fluid, causing it to pressurize the expandable membrane 217.

In some embodiments, the pressure chamber 232 may be configured as an electrolytic or electrochemical cell for conducting electrolysis. In some embodiments, the pressurization chamber comprises an anode and a cathode, and hydrogen may be produced at the cathode and oxygen may be produced at the anode when electrical current is passed between the anode and the cathode. For instance, the half reactions for typical alkaline aqueous electrolysis is expressed below by Equations 1A and 1B:

Cathode: 4H₂O+4e⁻→4OH⁻+H₂ Equation 1A:

Anode: 4OH⁻→2H₂O+O₂+4e⁻ Equation 1B:

Equation 2 reflects the overall reaction of traditional alkaline water splitting:

H₂O→H₂+½O₂ Equation 3:

The theoretical minimum thermodynamic potential for water electrolysis at STP is 1.23 Volts. According to some embodiments, the power source 245 as described above may be used to provide power to the electrodes for conducting electrolysis. For instance, in some embodiments, a button cell battery may be used. As will be understood, the type and size of power source used to conduct electrolysis may depend on the application. In some instances, a larger capacity battery may be necessary.

The pressurization chamber 232 may also be pressurized by gases through other methods. For instance, a cartridge containing pressurized gas, such as CO₂, may be used to pressurize the pressurization fluid, or may be released into the pressure chamber 232 to function as the pressurization fluid itself. The activation signal 470 may function to open a valve on the gas cartridge, thereby allowing the pressurized gas to enter the pressure chamber 232.

The controller 240 may send the activation signal 470 based on a variety of different factors. As shown in FIG. 4A, the presence of a sample 465 that has entered the assay chamber 225 may be detected by the controller 240 and thereby generate the activation signal 470. In other embodiments, the activation signal 470 may be sent on a predetermined period of time, such as on a daily or weekly basis. In other embodiments, the activation signal 470 may be sent based on user input received by the controller. For instance, a user, such as a systems manager, may request that the controller generate the activation signal 470. The user may input the request based on any one of a number of different factors, such as intelligence or other monitoring protocols.

In some embodiments, the sample 465 may enter the assay chamber 225 via gravitational forces. For instance, airborne particles may enter the assay chamber 225 through gravity and rest on a bottom surface or platform of the chamber. In other embodiments, a user may place a sample into the assay chamber 225 directly, for example, using a swab. In some instances, the user may also manually generate the activation signal 470. The assay chamber 225 may include one or more features, such as a filter for filtering out unwanted particles besides the desired sample, or a vapor trap for collecting and/or holding the sample. In some embodiments, the sensor may include one or more components that assist in taking air samples. For instance, a fan, a blower, or other air moving device may be implemented into the sensor system for purposes of obtaining samples. Air circulated by the fan from the external environment may be used for sampling purposes.

Once the unit cell is activated, at act 310, pressure builds in the pressure chamber 232 to pressurize the pressure chamber 232. The pressure chamber 232 is an enclosed structure, and may be pressurized by one or more gases generated from an electrolysis reaction, as described above, or the gas may be introduced into the pressure chamber 232 from a gas cartridge that contains one or more pressurized gases. The increase in pressure within the pressure chamber 232 causes the expandable membrane 217 to deform (act 315). The expandable membrane 217 deforms such that it expands into the activation chamber 112 and applies pressure to the activation fluid 110 at act 320. At act 325, the first membrane 115a is ruptured by pressure exerted by the activation fluid. In some instances, a portion of the first membrane 115a may rupture. The first membrane 115a may be constructed such that a weaker, more easily ruptured material may construct an inner portion of the membrane and the outer circumference or outer area of the membrane may be constructed from a stronger material that is not designed to rupture. This type of construction may allow for a tighter seal and more hermetic configuration. In other embodiments, larger portions of the membrane or the entire membrane may rupture.

According to some embodiments, the activation chamber 112 may comprise a first portion having a first diameter, and a second portion having a second diameter, and the first diameter may be larger than the second diameter. For example, referring to FIG. 4B, a first portion of the activation chamber (labeled “A1”) may have a larger diameter than a second portion (labeled “A2”). The second portion of the activation chamber 112 may be positioned adjacent to the biological chamber 122 and the first membrane 115a. The smaller diameter of the second portion may function to increase pressure exerted onto the first membrane 115a. This configuration may allow for the pressure required to rupture the first membrane 115a to be less than a pressure that would be required to rupture the first membrane if the diameter of the activation chamber was the same throughout the chamber.

Once the first membrane 115a ruptures, at act 330 activation fluid 110 may be introduced to the biological chamber 122, as shown in FIG. 4B. At least a portion of the activation fluid 110 may be introduced to the biological chamber 122. The activation fluid 110 may be at least one of a culture medium and a reaction medium as described above. For example, the biological component 120 may be a biotic and the activation fluid 110 may be a culture medium for the metabolic activity and/or growth of the biotic. In other examples, the biological component 120 may be an abiotic and the activation fluid 110 may be a reaction medium, such as a buffer solution, that re-suspends the abiotic. According to certain embodiments, the activation fluid 110 may function as both a culture medium and a reaction medium, and may therefore be capable of reconstituting both biotic and abiotic biological components. For instance, one or more components of the activation fluid 110 may be used by biotic and abiotic biological components. In some embodiments, the activation fluid 110 may include one or more components that function to reconstitute both microbes, such as bacteria, and DNA molecules, such as DNAzymes.

According to some embodiments, the walls of the biological chamber may be “coated” with dried biological component. For instance, the dried biological component may be sprayed or deposited onto the walls of the biological chamber. In at least one embodiment, the volume of the biological chamber may be at least partially filled with the biological component. In some embodiments, the dried biological components may be deposited into the biological chamber as a loose powder or compressed pellet. In other embodiments, the biological component is introduced into the biological chamber in a liquid state, and then undergoes a drying process while in the biological chamber.

According to some embodiments, two different biological components may be included in the biological chamber. For instance, a combination of both a lyophilized bacteria and a lyophilized DNAzyme may be included in the biological chamber.

In accordance with some embodiments, multiple biological components may be included in the biological chamber. Depending on the application, the number of biological components included in the biological chamber may be limited by one or more factors. For example, in some embodiments, the types of signal outputs may limit the number and types of biological components that can be included in a single biological chamber. For instance, in some instances, a maximum number of fluorescent signal outputs and/or a maximum number of luminescent signal outputs may determine how many biological components can be included in the biological chamber. According to some applications, multiple biological components may utilize the same signal output, and therefore the number of biological components included in the biological chamber may not necessarily be limited. This may be useful in applications where the identity, e.g., chemical species of the target analyte is not of concern since multiple species will exhibit the property of the target analyte. For instance, several target analytes that exhibit explosive behavior may cause several different biological components to fluoresce or luminesce at a characteristic wavelength.

From act 330, the process continues to act 335, which is shown in FIG. 3B and includes combining the activation fluid 110 with the biological component 120 contained in the biological chamber 122. The combined activation fluid 110 and biological component 120 form reconstituted biological component 460 (see FIG. 4C). The amount of activation fluid 110 introduced to the biological chamber 122 may be a volumetric amount sufficient to reconstitute the biological component such that it functions to detect a target analyte and emit or otherwise express or indicate a response based on the detection. The activation chamber 112 may be configured to provide a sufficient amount of activation fluid 110 to reconstitute the biological chamber 122. In some instances, the activation chamber 112 may be configured to provide a portion of the activation fluid 110 to the biological chamber 122. The biological chamber 122 may be configured to provide an adequate amount of biological component 120 to positively detect one or more target analytes and to allow for the activation fluid 110 to properly reconstitute the biological component 120. In some embodiments, the activation chamber 112 is sized to be larger than the biological chamber 122. The volumes of each of the chambers in the sensor system may vary depending on the application and the size of the overall device.

According to some embodiments, the activation fluid may hydrate the dried biological component for a period of time such that the biological component is reconstituted to a degree sufficient to react with the target analyte. In some instances, full reconstitution may occur in the biological chamber, but in other instances partial reconstitution may occur in the biological chamber and the remainder of the reconstitution process may occur in the assay chamber. In some instances, reconstitution may take only a few seconds or minutes, whereas in some applications the process may take several hours.

At act 340, the biological chamber is pressurized. According to one embodiment, the pressure generated at step 310 in the pressure chamber is sufficient to pressurize not only the activation fluid 110, but also the reconstituted biological component 460. In other embodiments, the pressure chamber 132 continues to pressurize after the first membrane 115a is ruptured, causing the expandable membrane 217 to continue to expand and pressurize the activation fluid 110, which continues to enter into the biological chamber 122 and combine with the biological component 120 to form reconstituted biological component 160. Either one of these scenarios may cause pressure to continue to build within the biological chamber 122 until the second membrane 115b ruptures.

At act 345, the second membrane 115b is ruptured by the reconstituted biological component 460, as shown in FIG. 4C. According to some embodiments, the second membrane 115b may be constructed in a similar manner as described above in reference to the first membrane 115a. For instance, a center portion of the second membrane 115b may be constructed from weaker material that is designed to rupture, whereas an outer region attached to the sidewalls or that extends into the housing is constructed from a stronger material that may be designed to seal or is otherwise configured to not rupture. In some embodiments, the first and second membranes 115a and 115b are configured to be substantially the same and therefore are configured to rupture under the same amount of exerted pressure. In other embodiments, the first membrane 115a may rupture at a different pressure than the second membrane 115b. For instance, the first membrane 115a may rupture at a higher or lower pressure than the second membrane 115b.

At act 350, the reconstituted biological component 460 is introduced to the assay chamber 350, as shown in FIG. 4C, through the ruptured second membrane 115b. In some embodiments, the reconstituted biological component 460 is introduced directly into the assay chamber 225. In other embodiments, the reconstituted biological component 460 may be introduced to the assay chamber 225 through a channel 227, as shown in FIG. 4C.

Once the reconstituted biological component 460 enters the assay chamber 225, it contacts the sample 465. The presence of a target analyte in the sample 465 may trigger the reconstituted biological component 460 to exhibit electrical or optical characteristics, which can be detected and analyzed by the analysis device in act 235. The analysis device 235 is therefore in communication with the reconstituted biological component 460 that contacts the sample, as indicated by the dashed line in FIG. 4D. For instance, as described above, the reconstituted biological component 460 may pass through a channel (not shown) to the analysis device, where the sample is measured and analyzed. In other embodiments, the assay chamber 225 contains one or more components of the analysis device 235, such as sensors and/or electrical circuitry that are in communication with a processor that controls and interprets data collected from the sensors and/or electrical circuitry.

The analysis device 235 may send an output signal 472 responsive to determining the presence or absence of the target analyte to a receiving device, such as the controller 240. The output signal 472 may be transmitted to the controller 240 immediately after analysis is performed, or the data may be stored and transmitted when requested by the controller 240 at a later time. For example, the reaction between the biological component and the target analyte may require a certain amount of time before an optical or electrical characteristic is exhibited by the biological component. In some embodiments, this reaction may happen nearly instantaneously, whereas in other embodiments, the reaction may take several minutes or hours. For instance, in some embodiments, bacteria may exhibit optical characteristics after a couple of hours. In other embodiments, DNAzymes may exhibit optical characteristics after several minutes. Therefore, the output signal 472 may be delayed for a period of time to allow the biological component to properly react. The output signal 472 may be transmitted through wireless communication methods. For instance, the output signal 472 may be transmitted via RF to an overhead aircraft that functions as a controller 240.

According to some embodiments, one or more of the chambers of the integrated sensor system 200, such as the biological chamber 122, may be configured as a microcavity. For instance, one or more of the chambers may be a cavity, channel, or other volumetric space that is sized to be less than about 500 microliters in total volume, although it is to be appreciated that larger and smaller volumes for the chambers are within the scope of this disclosure. According to one embodiment, the activation chamber 112 is sized to be less than 500 μL in volume and the biological chamber 122 is sized to be less than 10 μL in volume. However, as will be appreciated, smaller and larger volumes for the chambers are within the scope of this disclosure. For instance, in one embodiment, the activation chamber 112 has a size of about 150 μL, and the biological chamber 122 has a size of about 5 μL. In this instance, about 100 μL of the activation fluid may be introduced into the biological chamber 122.

In some embodiments, the chambers and/or conduits of the sensor system may be configured to facilitate capillary action, which may be utilized as a source of fluid flow during operation of the sensor. For instance, the A2 portion of the activation chamber 112, the biological chamber 122, or the channel 227 may be sized to promote or invoke capillary action of either the activation fluid 110 as it flow into and through the biological chamber, and/or the reconstituted biological component 460 as it flows into the assay chamber 225.

### Biological Component

According to some embodiments, the integrated sensor system comprises a biological component 120. In accordance with some embodiments, the biological component 120 may be a dried biological component. As used herein, the term “dried” refers to biological material that retains an amount of residual moisture that allows for the biological component 120 to be stored in the integrated sensor system for a desired period of time and remain viable upon reconstitution. In some embodiments, the biological component may retain less than 10% residual moisture, and in some instances, may retain less than 5% residual moisture by weight. For instance, a dried biological component that is a DNA molecule, such as DNAzyme, may retain less than 5% residual moisture. For dried biological component that is bacteria, e.g., bacterial spores, the residual moisture content may be much higher. In certain instances, drying may render the biological component to an “inactive” state. As explained further below, the biological component can be “reactivated” using an activation fluid. One example of a drying process includes freeze-drying the biological component. Freeze-drying typically involves rapidly reducing the temperature of the aqueous-containing biological component followed by a dewatering process conducted under reduced pressures. In certain instances, the biological component may be dried using air-drying or spray-drying techniques. The dried biological component may be stored for varying lengths of time depending on their composition.

The amount of biological component included in the biological chamber may vary depending on several factors, including the application, the type of biological component, and the configuration of the sensor device itself. In some embodiments, several milligrams of biological component may be included in the biological chamber. In some instances about 10 mg of material may be used, but it will be appreciated that other quantities, including smaller and larger quantities, are within the scope of this disclosure.

According to some embodiments, the biological component 120 is biotic, as described above. For instance, the biological component 120 may be a bacteria or yeast. According to some embodiments, the biological component comprises a virus. The virus may be any single virus, e.g., a bacteriophage infecting a bacteria. In some embodiments, the bacteriophage may infect and lyse its host bacteria. In some embodiments, the bacteriophage may insert into the bacterial chromosome. According to another embodiment, the biological component comprises fungi. For example, the genetically engineered material may comprise mold.

The biological component, such as bacteria, e.g., bacterial spores, may be dried using a freeze-drying technique. In embodiments, the dried biological component is a “dormant” form of the biological component. In the dried state, no metabolic or reproductive activity occurs, but genetic material and information is preserved. The dried biological component 120 may remain viable for extended periods of time in, depending on one or more factors, including the type of biological component, the concentration of biological component, and external factors, such as the environment. For instance, the biological component 120 may be left in the dried state for periods of time exceeding one month, and in some instances may be left in the dried state for several months, and may even remain viable after many years. The storage conditions may affect the amount of time that the biological component may be left in the dried state. For instance, dry and stable conditions may extend the amount of time that the biological component may be stored in the dried state. In some instances, lyophilized bacteria may remain viable for many years, whereas lyophilized DNA molecules, such as DNAzymes, may remain viable for many months. The type of application may also affect the length of storage time. For instance, lower detectable limits for a target analyte may allow for longer storage periods.

In some embodiments, the biological component is bacteria that undergoes a freeze-drying or lyophilization process that renders the bacteria into a dried state that allows the bacteria to be deployable into a biosensor. According to some embodiments, bacterial spores are formed and then subjected to the drying process. The spores may then be used in the biological chamber and used as the biological composition.

In some embodiments, bacteria are subjected to sporulation conditions, as understood by those skilled in the art. For instance, the process may first include growing the bacteria in a vegetative state and transferring the bacteria to sporulation medium which promotes formation of a spore, e.g., an endospore. Once the spores are re-introduced to an activation fluid such as a culture medium that supports their metabolic activity and/or growth, the spores re-establish metabolic activity and/or cell division.

The dried or lyophilized bacteria do not require food and are smaller in size than their pre-dried form.

In some embodiments, the bacterial spores can be enriched, e.g., purified away from their vegetative counterparts, which allows for a higher viability per unit volume than sporulation alone. For instance, vegetative bacteria can be killed, e.g., with lysozyme, heat, somotic shock, or chemical exposure. The spores can then be collected from the cell debris, e.g., by centrifugation or filtration. In some embodiment, the biological component comprises at least 50%, 60%, 70%, 80%, or 90% bacterial spores, e.g., prior to lyophilization.

The dried bacteria can also be resistant to desiccation, extreme heat, such as temperatures that are greater than 90° C., and to environmental threats, such as radiation and chemical threats, such as exposure to certain chemicals.

In accordance with some embodiments, freeze-drying the bacteria may comprise cooling the bacteria to a temperature in a range between about −10° C. to about −100° C. The bacteria may be cooled at a rate of between about 0.5° C./min to about 100° C./minute. The bacteria may be placed in a freeze or freeze-drying chamber for the freezing process, or may be exposed to liquid nitrogen.

In some embodiments, the biological component comprises a bacterial spore, a protozoan cyst, a cyanobacterial akinete, or a fungal exospore.

In some embodiments, nucleic acid (e.g., DNAzyme) or bacterial spores are subjected to lyophilization. Lyophilization generally exposes a substance to low pressure and low temperature. More specifically, lyophilization can involve stages of pretreatment (e.g., adding a lyoprotectant), freezing (e.g., using dry ice or liquid nitrogen, to a temperature below the triple point of water, about 0.01° C.), and drying under conditions that allow sublimation. The drying phase can include a first step of exposing the sample to partial vacuum (e.g., a few millibars), at a temperature of about the triple point of water, resulting in about 95% of water being lost. The drying phase can also include a second step of removing remaining liquid water molecules, e.g., at a higher temperature and lower pressure than the first phase.

In some embodiments, the biological component is a genetically engineered bacterium, e.g., a bacterial spore. In embodiments, the biological component comprises a spore-forming bacterium comprising a riboswitch (e.g., a fluoride-binding riboswitch, e.g., a riboswitch having a sequence of:

(SEQ ID NO: 1)
ATGTAATAATTATAGGCGATGGAGTTCGCCATAAACGCTGCTTAGCTAAT
GACTCCTACCAGTATCACTACTGGTAGGAGTCTATTTTTTTGAGCAAGCT
ATTTAAAGAGG

or a riboswitch having a similar structure). In embodiments, the riboswitch has no more than 5, 4, 3, 2, or 1 substitutions, deletions, or insertions relative to SEQ ID NO: 1. In embodiments, the riboswitch has a similar structure to the structure shown in FIG. 1A or 1B of Baker et al., “Widespread Genetic Switches and Toxicity Resistance Proteins for Fluoride” Science. 2012 Jan. 13; 335(6065): 233-235, which is herein incorporated by reference in its entirety. In embodiments, the riboswitch has the same number and length of double stranded regions and the same number and length of single stranded regions as the riboswitches shown in FIG. 1A or 1B of Baker et al. In embodiments, the riboswitch modulates accessibility of a ribosome binding site.

In embodiments, the nucleic acid comprises a B. subtilis lysine promoter driving expression of the riboswitch, e.g., as shown below:

(SEQ ID NO: 2)
AAAAATAATGTTGTCCTTTTAAATAAGATCTGATAAAATGTGAACTAAAT
GTAATAATTATAGGCGATGGAGTTCGCCATAAACGCTGCTTAGCTAATGA
CTCCTACCAGTATCACTACTGGTAGGAGTCTATTTTTTTGAGCAAGCTAT
TTAAAGAGG

In embodiments, the riboswitch-lacZ containing construct for integration into the amyE locus has the following sequence:

(SEQ ID NO: 3)

| | |
|---|---|
| 1 | aattcttgaa gacgaaaggg cctcgtgata cgcctatttt |
| | tataggttaa tgtcatgata |
| 61 | ataatggttt cttagacgtc aggtggcact tttcggggaa |
| | atgtgcgcgg aacccctatt |
| 121 | tgtttatttt tctaaataca ttcaaatatg tatccgctca |
| | tgagacaata accctgataa |
| 181 | atgcttcaat aatattgaaa aaggaagagt atgagtattc |
| | aacatttccg tgtcgccctt |
| 241 | attccctttt ttgcggcatt ttgccttcct gtttttgctc |
| | acccagaaac gctggtgaaa |
| 301 | gtaaaagatg ctgaagatca gttgggtgca cgagtgggtt |
| | acatcgaact ggatctcaac |
| 361 | agcggtaaga tccttgagag ttttcgcccc gaagaacgtt |
| | ttccaatgat gagcactttt |
| 421 | aaagttctgc tatgtggcgc ggtattatcc cgtgttgacg |
| | ccgggcaaga gcaactcggt |
| 481 | cgccgcatac actattctca gaatgacttg gttgagtact |
| | caccagtcac agaaaagcat |
| 541 | cttacggatg gcatgacagt aagagaatta tgcagtgctg |
| | ccataaccat gagtgataac |
| 601 | actgcggcca acttacttct gacaacgatc ggaggaccga |
| | aggagctaac cgcttttttg |
| 661 | cacaacatgg gggatcatgt aactcgcctt gatcgttggg |
| | aaccggagct gaatgaagcc |
| 721 | ataccaaacg acgagcgtga caccacgatg cctgcagcaa |
| | tggcaacaac gttgcgcaaa |
| 781 | ctattaactg gcgaactact tactctagct tcccggcaac |
| | aattaataga ctggatggag |
| 841 | gcggataaag ttgcaggacc acttctgcgc tcggcccttc |
| | cggctggctg gtttattgct |
| 901 | gataaatctg gagccggtga gcgtgggtct cgcggtatca |
| | ttgcagcact ggggccagat |
| 961 | ggtaagccct cccgtatcgt agttatctac acgacgggga |
| | gtcaggcaac tatggatgaa |
| 1021 | cgaaatagac agatcgctga gataggtgcc tcactgatta |
| | agcattggta actgtcagac |
| 1081 | caagtttact catatatact ttagattgat ttaaaacttc |
| | atttttaatt taaaaggatc |
| 1141 | taggtgaaga tcctttttga taatctcatg accaaaatcc |
| | cttaacgtga gttttcgttc |
| 1201 | cactgagcgt cagaccccgt agaaaagatc aaaggatctt |
| | cttgagatcc tttttttctg |
| 1261 | cgcgtaatct gctgcttgca aacaaaaaaa ccaccgctac |
| | cagcggtggt ttgtttgccg |
| 1321 | gatcaagagc tatcaactct ttttccgaag gtaactggct |
| | tcagcagagc gcagatacca |
| 1381 | aatactgtcc ttctagtgta gccgtagtta ggccaccact |
| | tcaagaactc tgtagcaccg |
| 1441 | cctacatacc tcgctctgct aatcctgtta ccagtggctg |
| | ctgccagtgg cgataagtcg |
| 1501 | tgtcttaccg ggttggactc aagacgatag ttaccggata |
| | aggcgcagcg gtcgggctga |
| 1561 | acggggggtt cgtgcacaca gcccagcttg gagcgaacga |
| | cctacaccga actgagatac |
| 1621 | ctacagcgtg agctatgaga aagcgccacg cttcccgaag |
| | ggagaaaggc ggacaggtat |
| 1681 | ccggtaagcg gcagggtcgg aacaggagag cgcacgaggg |
| | agcttccagg gggaaacgcc |
| 1741 | tggtatcttt atagtcctgt cgggtttcgc cacctctgac |
| | ttgagcgtcg atttttgtga |
| 1801 | tgctcgtcag gggggcggag cctatggaaa aacgccagca |
| | acgcggcccg acctcgagct |
| 1861 | ggatacttcc cgtccgccag ggggacatgc cggcgatgct |
| | gaaggtcgcg cgcattcccg |
| 1921 | atgaagaggc cggttaccgc ctgtttgagg atatagtaat |
| | ctttctaaat agctttggat |
| 1981 | tggaggagta tggccactaa tactaagttc agctaataaa |
| | aaaatttgct aaagaactcc |
| 2041 | agaaaagtaa gcacctgtta ttgcaataaa attagcctaa |
| | ttgagagaag tttctataga |
| 2101 | atttttcata tacttaacga gtgctttcac ctttgaatat |
| | agtccttccc acttatcatc |
| 2161 | acactctccc cgatagcctt ttctagctat atccagtaaa |
| | gttacatgct ctttaggtaa |
| 2221 | aagaggtata gcccattctg cagcgacatc tttcgaggta |
| | atttcaccag tagtcactgt |
| 2281 | ttgccacatt cgagctaggg ttaaaattac attacgctca |
| | tcacctttta tcccctcaat |
| 2341 | tagttctggc aaagaatcct taattgctct tcgaatatct |
| | gtcaaaggta cggagacaag |
| 2401 | tatacttgaa gaatcaggac caaatagaga aatactattc |
| | tttcttgctt gtgctaaaac |
| 2461 | aatagccaaa tcaggatcat agcttggttc ctgaatttgt |
| | ccattctcaa attcacccct |
| 2521 | gagccactca ccgtatataa attctctttt tggaggatat |
| | tgccaaggga caacttcact |
| 2581 | cctatttata accgtaactt caagtggtct aacagaatcc |
| | gtatttccaa tctttcctga |
| 2641 | tatagtcatt agtctttctg ttagtttttt tcgagttaat |
| | tgaggtaaac tatgattcac |
| 2701 | gacgactaga acatctacat cgctgttaat gcgtaaacca |
| | ccatttactg ctgaaccaaa |
| 2761 | tagatatact ccaactattg aacttccaaa taaatctttt |
| | acgattttta atgtttgaat |
| 2821 | cgcttgattt ggtatttttc cgttaatcaa attgctcatg |
| | atttcacctc gttgattatg |
| 2881 | ttcatataaa gtttatattg atactcaatt tacttaccct |
| | agattggaca tatacttaaa |
| 2941 | ttactgttca ataaagctga ccgttagcgt ttaagtacat |
| | cctttcacaa tttgtctaca |
| 3001 | gattaataat tattctttat tatacagatc gatcctctag |
| | acctaggcct taagatctga |
| 3061 | tcatatgcat ccgcgggccc gggttaacgc gtaatccatg |
| | gatcaagaga caggatgagg |
| 3121 | atcgtttcgc atgattgaac aagatggatt gcacgcaggt |
| | tctccggtgc cctgaatgaa |
| 3181 | ctgcagaaag agctggtagt tggcgcactg ttcgaagaac |
| | tgccgatgtc cagtaagatt |
| 3241 | cttactatgc tggttgaacc ggatgctggt aaagctactt |
| | gggttgctgc ttctacttat |
| 3301 | ggtaccgata caactactgg tgaggaagtt aaaggagctc |
| | ttaaagaaat ccacttcagt |
| 3361 | aggtggcccg gctccatgca ccgcgacgca acgcggggag |
| | gcagacaagg tatagggcgg |
| 3421 | cgcctacaat ccatgccaac ccgttccatg tgctcgccga |
| | ggcggcataa atcgccgtga |
| 3481 | cgatcagcgg tccagtgatc gaagttaggc tggtaagagc |
| | cgcgagcgat ccttgaagct |
| 3541 | gtccctgatg gtcgtcatct acctgcctgg acagcatggc |
| | ctgcaacgcg ggcatcccga |
| 3601 | tgccgccgga agcgagaaga atcataatgg ggaaggccat |
| | ccagcctcgc gtcgcgacta |
| 3661 | agaaaatgcc gtcaaatccg ctcgccatga cttcactaac |
| | gatgcctttg aaaatcttca |
| 3721 | agttcttttc tactaattca aggcgtgtct caccaggttt |
| | ttggtttgct ccggcgcaaa |
| 3781 | tgcagacaat atcagcatcc ttgcagggta tgtttctctt |
| | tgatgtcttt ttgtttgtga |
| 3841 | agtatttcac atttatattg tgcaacactt cacaaacttt |
| | tgcaagagaa aagttttgtc |
| 3901 | tgatttatga acaaaaaaga aaccatcatt gatggtttct |
| | ttcggtaagt cccgtctagc |
| 3961 | cttgccctca atggggaaga gaaccgctta agcccgagtc |
| | attatataaa ccatttagca |
| 4021 | cgtaatcaaa gccaggctga ttctgaccgg gcacttgggc |
| | gctgccatta ttaaaaatca |
| 4081 | cttttgcgtt ggttgtatcc gtgtccgcag gcagcgtcag |
| | cgtgtaaatt ccgtctgcat |
| 4141 | ttttagtcat tggttttcca ggccaagatc cggtcaattc |
| | aattactcgg ctcccatcat |
| 4201 | gtttatagat ataagcattt acctggctcc aatgattcgg |
| | attttgatag ccgatggttt |
| 4261 | tggccgacgc tggatctctt ttaacaaaac tgtatttctc |
| | ggtcctcgtt acaccatcac |
| 4321 | tgttcgttcc ttttaacatg atggtgtatg ttttgccaaa |
| | ttggatctcc ttttccgatt |
| 4381 | gtgaattgat ctccatcctt aaacgcctgt cgtctggtcc |
| | attattgatt tgataaacgg |
| 4441 | cttttgttgt attcgcatct gcacgcaagg taatcgtcag |
| | ttgatcattg aaagaatgtg |
| 4501 | ttacacctgt tttgtaattc tcaaggaaaa catgaggcgc |
| | ttttgcaata tcatcaggat |
| 4561 | aaagcacagc tacagacctg gcattgatcg tgcctgtcag |
| | tttaccatcg ttcacttgaa |
| 4621 | atgaacccgc tccagcttta ttgtcatacc tgccatcagg |
| | caattttgtt gccgtattga |
| 4681 | tagagacaga ggatgaacct gcatttgcca gcacaacgcc |
| | atgtgagccg cgctgattca |
| 4741 | taaatatctg gttgtttcca ttcgggttcg agagttcctc |
| | aggctgtcca gccatcacat |
| 4801 | tgtgaaatct attgaccgca gtgatagcct gatcttcaaa |
| | taaagcactc ccgcgatcgc |
| 4861 | ctatttggct tttccccggg aacctcacac catttccgcc |
| | tccctcaggt ctggaaaaga |
| 4921 | aaagaggcgt actgcctgaa cgagaagcta tcaccgccca |
| | gcctaaacgg atatcatcat |
| 4981 | cgctcatcca tgtcgacgct ctcccttatg cgactcctgc |
| | attaggaagc agcccagtag |
| 5041 | taggttgagg ccgttgagca ccgccgccgc aaggaatggt |
| | gcatgcaagg agatggcgcc |
| 5101 | caacagtccc ccggccacgg ggcctgccac catacccacg |
| | ccgaaacaag cgctcatgag |
| 5161 | cccgaagtgg cgagcccgat cttccccatc ggtgatgtcg |
| | gcgatatagg cgccagcaac |
| 5221 | cgcacctgtg gcgccggtga tgccggccac gatgcgtccg |
| | gcgtagagga tctggagctg |
| 5281 | taatataaaa accttcttca actaacgggg caggttagtg |
| | acattagaaa accgactgta |
| 5341 | aaaagtacag tcggcattat ctcatattat aaaagccagt |
| | cattaggcct atctgacaat |
| 5401 | tcctgaatag agttcataaa caatcctgca tgataaccat |
| | cacaaacaga atgatgtacc |
| 5461 | tgtaaagata gcggtaaata tattgaatta cctttattaa |
| | tgaattttcc tgctgtaata |
| 5521 | atgggtagaa ggtaattact attattattg atatttaagt |
| | taaacccagt aaatgaagtc |
| 5581 | catggaataa tagaaagaga aaaagcattt tcaggtatag |
| | gtgttttggg aaacaatttc |
| 5641 | cccgaaccat tatatttctc tacatcagaa aggtataaat |
| | cataaaactc tttgaagtca |
| 5701 | ttctttacag gagtccaaat accagagaat gttttagata |
| | caccatcaaa aattgtataa |
| 5761 | agtggctcta acttatccca ataacctaac tctccgtcgc |
| | tattgtaacc agttctaaaa |
| 5821 | gctgtatttg agtttatcac ccttgtcact aagaaaataa |
| | atgcagggta aaatttatat |
| 5881 | ccttcttgtt ttatgtttcg gtataaaaca ctaatatcaa |
| | tttctgtggt tatactaaaa |
| 5941 | gtcgtttgtt ggttcaaata atgattaaat atctcttttc |
| | tcttccaatt gtctaaatca |
| 6001 | attttattaa agttcatttg atatgcctcc taaattttta |
| | tctaaagtga atttaggagg |
| 6061 | cttacttgtc tgctttcttc attagaatca atcctttttt |
| | aaaagtcaat attactgtaa |
| 6121 | cataaatata tattttaaaa atatcccact ttatccaatt |
| | ttcgtttgtt gaactaatgg |
| 6181 | gtgctttagt tgaagaataa agaccacatt aaaaaatgtg |
| | gtcttttgtg tttttttaaa |
| 6241 | ggatttgagc gtagcgaaaa atccttttct ttcttatctt |
| | gataataagg gtaactattg |
| 6301 | ccgatgataa gctgtcaaac atgagaattc aaaaataatg |
| | ttgtcctttt aaataagatc |
| 6361 | tgataaaatg tgaactaaat gtaataatta taggcgatgg |
| | agttcgccat aaacgctgct |
| 6421 | tagctaatga ctcctaccag tatcactact ggtaggagtc |
| | tatttttttg agcaagctat |
| 6481 | ttaaagaggg gatccccagc ttgttgatac actaatgctt |
| | ttatataggg aaaaggtggt |
| 6541 | gaactactgt ggaagttact gacgtaagat tacgggtcga |
| | ccgggaaaac cctggcgtta |
| 6601 | cccaacttaa tcgccttgca gcacatcccc ctttcgccag |
| | ctggcgtaat agcgaagagg |
| 6661 | cccgcaccga tcgcccttcc caacagttgc gcagcctgaa |
| | tggcgaatgg cgctttgcct |
| 6721 | ggtttccggc accagaagcg gtgccggaaa gctggctgga |
| | gtgcgatctt cctgaggccg |
| 6781 | atactgtcgt cgtcccctca aactggcaga tgcacggtta |
| | cgatgcgccc atctacacca |
| 6841 | acgtaaccta tcccattacg gtcaatccgc cgtttgttcc |
| | cacggagaat ccgacgggtt |
| 6901 | gttactcgct cacatttaat gttgatgaaa gctggctaca |
| | ggaaggccag acgcgaatta |
| 6961 | tttttgatgg cgttaactcg gcgtttcatc tgtggtgcaa |
| | cgggcgctgg gtcggttacg |
| 7021 | gccaggacag tcgtttgccg tctgaatttg acctgagcgc |
| | atttttacgc gccggagaaa |
| 7081 | accgcctcgc ggtgatggtg ctgcgttgga gtgacggcag |
| | ttatctggaa gatcaggata |
| 7141 | tgtggcggat gagcggcatt ttccgtgacg tctcgttgct |
| | gcataaaccg actacacaaa |
| 7201 | tcagcgattt ccatgttgcc actcgcttta atgatgattt |
| | cagccgcgct gtactggagg |
| 7261 | ctgaagttca gatgtgcggc gagttgcgtg actacctacg |
| | ggtaacagtt tctttatggc |
| 7321 | agggtgaaac gcaggtcgcc agcggcaccg cgcctttcgg |
| | cggtgaaatt atcgatgagc |
| 7381 | gtggtggtta tgccgatcgc gtcacactac gtctgaacgt |
| | cgaaaacccg aaactgtgga |
| 7441 | gcgccgaaat cccgaatctc tatcgtgcgg tggttgaact |
| | gcacaccgcc gacggcacgc |
| 7501 | tgattgaagc agaagcctgc gatgtcggtt tccgcgaggt |
| | gcggattgaa aatggtctgc |
| 7561 | tgctgctgaa cggcaagccg ttgctgattc gaggcgttaa |
| | ccgtcacgag catcatcctc |
| 7621 | tgcatggtca ggtcatggat gagcagacga tggtgcagga |
| | tatcctgctg atgaagcaga |
| 7681 | acaactttaa cgccgtgcgc tgttcgcatt atccgaacca |
| | tccgctgtgg tacacgctgt |
| 7741 | gcgaccgcta cggcctgtat gtggtggatg aagccaatat |
| | tgaaacccac ggcatggtgc |
| 7801 | caatgaatcg tctgaccgat gatccgcgct ggctaccggc |
| | gatgagcgaa cgcgtaacgc |
| 7861 | gaatggtgca gcgcgatcgt aatcacccga gtgtgatcat |
| | ctggtcgctg gggaatgaat |
| 7921 | caggccacgg cgctaatcac gacgcgctgt atcgctggat |
| | caaatctgtc gatccttccc |
| 7981 | gcccggtgca gtatgaaggc ggcggagccg acaccacggc |
| | caccgatatt atttgcccga |
| 8041 | tgtacgcgcg cgtggatgaa gaccagccct tcccggctgt |
| | gccgaaatgg tccatcaaaa |
| 8101 | aatggctttc gctacctgga gagacgcgcc cgctgatcct |
| | ttgcgaatac gcccacgcga |
| 8161 | tgggtaacag tcttggcggt ttcgctaaat actggcaggc |
| | gtttcgtcag tatccccgtt |
| 8221 | tacagggcgg cttcgtctgg gactgggtgg atcagtcgct |
| | gattaaatat gatgaaaacg |
| 8281 | gcaacccgtg gtcggcttac ggcggtgatt ttggcgatac |
| | gccgaacgat cgccagttct |
| 8341 | gtatgaacgg tctggtcttt gccgaccgca cgccgcatcc |
| | agcgctgacg gaagcaaaac |
| 8401 | accagcagca gtttttccag ttccgtttat ccgggcaaac |
| | catcgaagtg accagcgaat |
| 8461 | acctgttccg tcatagcgat aacgagctcc tgcactggat |
| | ggtggcgctg gatggtaagc |
| 8521 | cgctggcaag cggtgaagtg cctctggatg tcgctccaca |
| | aggtaaacag ttgattgaac |
| 8581 | tgcctgaact accgcagccg gagagcgccg ggcaactctg |
| | gctcacagta cgcgtagtgc |
| 8641 | aaccgaacgc gaccgcatgg tcagaagccg ggcacatcag |
| | cgcctggcag cagtggcgtc |
| 8701 | tggcggaaaa cctcagtgtg acgctccccg ccgcgtccca |
| | cgccatcccg catctgacca |
| 8761 | ccagcgaaat ggatttttgc atcgagctgg gtaataagcg |
| | ttggcaattt aaccgccagt |
| 8821 | caggctttct ttcacagatg tggattggcg ataaaaaaca |
| | actgctgacg ccgctgcgcg |
| 8881 | atcagttcac ccgtgcaccg ctggataacg acattggcgt |
| | aagtgaagcg acccgcattg |
| 8941 | accctaacgc ctgggtcgaa cgctggaagg cggcgggcca |
| | ttaccaggcc gaagcagcgt |
| 9001 | tgttgcagtg cacggcagat acacttgctg atgcggtgct |
| | gattacgacc gctcacgcgt |
| 9061 | ggcagcatca ggggaaaacc ttatttatca gccggaaaac |
| | ctaccggatt gatggtagtg |
| 9121 | gtcaaatggc gattaccgtt gatgttgaag tggcgagcga |
| | tacaccgcat ccggcgcgga |
| 9181 | ttggcctgaa ctgccagctg gcgcaggtag cagagcgggt |
| | aaactggctc ggattagggc |
| 9241 | cgcaagaaaa ctatcccgac cgccttactg ccgcctgttt |
| | tgaccgctgg gatctgccat |
| 9301 | tgtcagacat gtataccccg tacgtcttcc cgagcgaaaa |
| | cggtctgcgc tgcgggacgc |
| 9361 | gcgaattgaa ttatggccca caccagtggc gcggcgactt |
| | ccagttcaac atcagccgct |
| 9421 | acagtcaaca gcaactgatg gaaaccagcc atcgccatct |
| | gctgcacgcg gaagaaggca |
| 9481 | catggctgaa tatcgacggt ttccatatgg ggattggtgg |
| | cgacgactcc tggagcccgt |
| 9541 | cagtatcggc ggaatttcag ctgagcgccg gtcgctacca |
| | ttaccagttg gtctggtgtc |
| 9601 | aaaaataata ataaccgggc aggccatgtc tgcccgtatt |
| | tcgcgtaagg aaatccatta |
| 9661 | tgtactatcg atcagaccag tttttaattt gtgtgtttcc |
| | atgtgtccag tttggaatac |
| 9721 | tcttaacctc attggaaatc gcggcataat cactggtggt |
| | atgattgatg accgcgtcaa |
| 9781 | caatgacctt tatgccatat tcttcagcgg ctgcacacat |
| | ttctttaaat tcttgttcag |
| 9841 | tacctaagta acggttgcca atttgatacg atgtcggctg |
| | atacagccag taccagttcg |
| 9901 | acatgctttt atctccttga ttcccttcct ttacttggtt |
| | aatcggagat gtctgaatgg |
| 9961 | ctgtatatcc tgcatcatga atatccttca tattgtgttt |
| | taacgtattg aacgaccaat |
| 10021 | tccatgcatg aagaatggtt ccgcttttga tcgacggtgc |
| | tgtaagctca ttcgatttgt |
| 10081 | tcgccgtttc agcactcgca gccgccggtc ctgccagaac |
| | caaatgaaac agcaataaaa |
| 10141 | atccagcgaa taacggcagt aaagaggttt tgaatcgttt |
| | tgcaaacatt cttgacactc |
| 10201 | cttatttgat tttttgaaga cttacttcgg agtcaaaaat |
| | ccctcttact tcattcttcc |
| 10261 | gcttcctcct ttcaaaccga tgtgaagact ggagaatttt |
| | gtt. |

In embodiments, the riboswitch-lux containing construct for integration into the amyE locus has the following sequence:

(SEQ ID NO: 4)

| | |
|---|---|
| 1 | agtagttcac caccttttcc ctatataaaa gcattagtgt |
| | atcaacaagc tggggatcct |
| 61 | agaagcttat cgaattctca tgtttgacag cttatcatcg |
| | gcaatagtta cccttattat |
| 121 | caagataaga aagaaaagga tttttcgcta cgctcaaatc |
| | ctttaaaaaa acacaaaaga |
| 181 | ccacattttt taatgtggtc tttattcttc aactaaagca |
| | cccattagtt caacaaacga |
| 241 | aaattggata aagtgggata tttttaaaat atatatttat |
| | gttacagtaa tattgacttt |
| 301 | taaaaaagga ttgattctaa tgaagaaagc agacaagtaa |
| | gcctcctaaa ttcactttag |
| 361 | ataaaaattt aggaggcata tcaaatgaac tttaataaaa |
| | ttgatttaga caattggaag |
| 421 | agaaaagaga tatttaatca ttatttgaac caacaaacga |
| | cttttagtat aaccacagaa |
| 481 | attgatatta gtgttttata ccgaaacata aaacaagaag |
| | gatataaatt ttaccctgca |
| 541 | tttattttct tagtgacaag ggtgataaac tcaaatacag |
| | cttttagaac tggttacaat |
| 601 | agcgacggag agttaggtta ttgggataag ttagagccac |
| | tttatacaat ttttgatggt |
| 661 | gtatctaaaa cattctctgg tatttggact cctgtaaaga |
| | atgacttcaa agagttttat |
| 721 | gatttatacc tttctgatgt agagaaatat aatggttcgg |
| | ggaaattgtt tcccaaaaca |
| 781 | cctatacctg aaaatgcttt ttctctttct attattccat |
| | ggacttcatt tactgggttt |
| 841 | aacttaaata tcaataataa tagtaattac cttctaccca |
| | ttattacagc aggaaaattc |
| 901 | attaataaag gtaattcaat atatttaccg ctatctttac |
| | aggtacatca ttctgtttgt |
| 961 | gatggttatc atgcaggatt gtttatgaac tctattcagg |
| | aattgtcaga taggcctaat |
| 1021 | gactggcttt tataatatga gataatgccg actgtacttt |
| | ttacagtcgg ttttctaatg |
| 1081 | tcactaacct gccccgttag ttgaagaagg tttttatatt |
| | acagctccag atcctctacg |
| 1141 | ccggacgcat cgtggccggc atcaccggcg ccacaggtgc |
| | ggttgctggc gcctatatcg |
| 1201 | ccgacatcac cgatggggaa gatcgggctc gccacttcgg |
| | gctcatgagc gcttgtttcg |
| 1261 | gcgtgggtat ggtggcaggc cccgtggccg ggggactgtt |
| | gggcgccatc tccttgcatg |
| 1321 | caccattcct tgcggcggcg gtgctcaacg gcctcaacct |
| | actactgggc tgcttcctaa |
| 1381 | tgcaggagtc gcataaggga gagcgtcgac atggatgagc |
| | gatgatgata tccgtttagg |
| 1441 | ctgggcggtg atagcttctc gttcaggcag tacgcctctt |
| | ttcttttcca gacctgaggg |
| 1501 | aggcggaaat ggtgtgaggt tcccggggaa aagccaaata |
| | ggcgatcgcg ggagtgcttt |
| 1561 | atttgaagat caggctatca ctgcggtcaa tagatttcac |
| | aatgtgatgg ctggacagcc |
| 1621 | tgaggaactc tcgaacccga atggaaacaa ccagatattt |
| | atgaatcagc gcggctcaca |
| 1681 | tggcgttgtg ctggcaaatg caggttcatc ctctgtctct |
| | atcaatacgg caacaaaatt |
| 1741 | gcctgatggc aggtatgaca ataaagctgg agcgggttca |
| | tttcaagtga acgatggtaa |
| 1801 | actgacaggc acgatcaatg ccaggtctgt agctgtgctt |
| | tatcctgatg atattgcaaa |
| 1861 | agcgcctcat gttttccttg agaattacaa aacaggtgta |
| | acacattctt tcaatgatca |
| 1921 | actgacgatt accttgcgtg cagatgcgaa tacaacaaaa |
| | gccgtttatc aaatcaataa |
| 1981 | tggaccagac gacaggcgtt taaggatgga gatcaattca |
| | caatcggaaa aggagatcca |
| 2041 | atttggcaaa acatacacca tcatgttaaa aggaacgaac |
| | agtgatggtg taacgaggac |
| 2101 | cgagaaatac agttttgtta aaagagatcc agcgtcggcc |
| | aaaaccatcg gctatcaaaa |
| 2161 | tccgaatcat tggagccagg taaatgctta tatctataaa |
| | catgatggga gccgagtaat |
| 2221 | tgaattgacc ggatcttggc ctggaaaacc aatgactaaa |
| | aatgcagacg gaatttacac |
| 2281 | gctgacgctg cctgcggaca cggatacaac caacgcaaaa |
| | gtgattttta ataatggcag |
| 2341 | cgcccaagtg cccggtcaga atcagcctgg ctttgattac |
| | gtgctaaatg gtttatataa |
| 2401 | tgactcgggc ttaagcggtt ctcttcccca ttgagggcaa |
| | ggctagacgg gacttaccga |
| 2461 | aagaaaccat caatgatggt ttcttttttg ttcataaatc |
| | agacaaaact tttctcttgc |
| 2521 | aaaagtttgt gaagtgttgc acaatataaa tgtgaaatac |
| | ttcacaaaca aaaagacatc |
| 2581 | aaagagaaac ataccctgca aggatgctga tattgtctgc |
| | atttgcgccg gagcaaacca |
| 2641 | aaaacctggt gagacacgcc ttgaattagt agaaaagaac |
| | ttgaagattt tcaaaggcat |
| 2701 | cgttagtgaa gtcatggcga gcggatttga cggcattttc |
| | ttagtcgcga cgcgaggctg |
| 2761 | gatggccttc cccattatga ttcttctcgc ttccggcggc |
| | atcgggatgc ccgcgttgca |
| 2821 | ggccatgctg tccaggcagg tagatgacga ccatcaggga |
| | cagcttcaag gatcgctcgc |
| 2881 | ggctcttacc agcctaactt cgatcactgg accgctgatc |
| | gtcacggcga tttatgccgc |
| 2941 | ctcggcgagc acatggaacg ggttggcatg gattgtaggc |
| | gccgccctat accttgtctg |
| 3001 | cctccccgcg ttgcgtcgcg gtgcatggag ccgggccacc |
| | tactgaagtg gatttcttta |
| 3061 | agagctcctt taacttcctc accagtagtt gtatcggtac |
| | cataagtaga agcagcaacc |
| 3121 | caagtagctt taccagcatc cggttcaacc agcatagtaa |
| | gaatcttact ggacatcggc |
| 3181 | agttcttcga acagtgcgcc aactaccagc tctttctgca |
| | gttcattcag ggcaccggag |
| 3241 | aacctgcgtg caatccatct tgttcaatca tgcgaaacga |
| | tcctcatcct gtctcttgat |
| 3301 | ccatggatta cgcgttaacc cgggcccgcg gatgcatatg |
| | atcagatctt aaggcctagg |
| 3361 | tctagaggat cgatctgtat aataaagaat aattattaat |
| | ctgtagacaa attgtgaaag |
| 3421 | gatgtactta aacgctaacg gtcagcttta ttgaacagta |
| | atttaagtat atgtccaatc |
| 3481 | tagggtaagt aaattgagta tcaatataaa ctttatatga |
| | acataatcaa cgaggtgaaa |
| 3541 | tcatgagcaa tttgattaac ggaaaaatac caaatcaagc |
| | gattcaaaca ttaaaaatcg |
| 3601 | taaaagattt atttggaagt tcaatagttg gagtatatct |
| | atttggttca gcagtaaatg |
| 3661 | gtggtttacg cattaacagc gatgtagatg ttctagtcgt |
| | cgtgaatcat agtttacctc |
| 3721 | aattaactcg aaaaaaacta acagaaagac taatgactat |
| | atcaggaaag attggaaata |
| 3781 | cggattctgt tagaccactt gaagttacgg ttataaatag |
| | gagtgaagtt gtcccttggc |
| 3841 | aatatcctcc aaaaagagaa tttatatacg gtgagtggct |
| | caggggtgaa tttgagaatg |
| 3901 | gacaaattca ggaaccaagc tatgatcctg atttggctat |
| | tgttttagca caagcaagaa |
| 3961 | agaatagtat ttctctattt ggtcctgatt cttcaagtat |
| | acttgtctcc gtacctttga |
| 4021 | cagatattcg aagagcaatt aaggattctt tgccagaact |
| | aattgagggg ataaaaggtg |
| 4081 | atgagcgtaa tgtaatttta accctagctc gaatgtggca |
| | aacagtgact actggtgaaa |
| 4141 | ttacctcgaa agatgtcgct gcagaatggg ctatacctct |
| | tttacctaaa gagcatgtaa |
| 4201 | ctttactgga tatagctaga aaaggctatc ggggagagtg |
| | tgatgataag tgggaaggac |
| 4261 | tatattcaaa ggtgaaagca ctcgttaagt atatgaaaaa |
| | ttctatagaa acttctctca |
| 4321 | attaggctaa ttttattgca ataacaggtg cttacttttc |
| | tggagttctt tagcaaattt |
| 4381 | ttttattagc tgaacttagt attagtggcc atactcctcc |
| | aatccaaagc tatttagaaa |
| 4441 | gattactata tcctcaaaca ggcggtaacc ggcctcttca |
| | tcgggaatgc gcgcgacctt |
| 4501 | cagcatcgcc ggcatgtccc cctggcggac gggaagtatc |
| | cagctcgagg tcgggccgcg |
| 4561 | ttgctggcgt ttttccatag gctccgcccc cctgacgagc |
| | atcacaaaaa tcgacgctca |
| 4621 | agtcagaggt ggcgaaaccc gacaggacta taaagatacc |
| | aggcgtttcc ccctggaagc |
| 4681 | tccctcgtgc gctctcctgt tccgaccctg ccgcttaccg |
| | gatacctgtc cgcctttctc |
| 4741 | ccttcgggaa gcgtggcgct ttctcatagc tcacgctgta |
| | ggtatctcag ttcggtgtag |
| 4801 | gtcgttcgct ccaagctggg ctgtgtgcac gaaccccccg |
| | ttcagcccga ccgctgcgcc |
| 4861 | ttatccggta actatcgtct tgagtccaac ccggtaagac |
| | acgacttatc gccactggca |
| 4921 | gcagccactg gtaacaggat tagcagagcg aggtatgtag |
| | gcggtgctac agagttcttg |
| 4981 | aagtggtggc ctaactacgg ctacactaga aggacagtat |
| | ttggtatctg cgctctgctg |
| 5041 | aagccagtta ccttcggaaa aagagttgat agctcttgat |
| | ccggcaaaca aaccaccgct |
| 5101 | ggtagcggtg gtttttttgt ttgcaagcag cagattacgc |
| | gcagaaaaaa aggatctcaa |
| 5161 | gaagatcctt tgatcttttc tacggggtct gacgctcagt |
| | ggaacgaaaa ctcacgttaa |
| 5221 | gggattttgg tcatgagatt atcaaaaagg atcttcacct |
| | agatcctttt aaattaaaaa |
| 5281 | tgaagtttta aatcaatcta aagtatatat gagtaaactt |
| | ggtctgacag ttaccaatgc |
| 5341 | ttaatcagtg aggcacctat ctcagcgatc tgtctatttc |
| | gttcatccat agttgcctga |
| 5401 | ctccccgtcg tgtagataac tacgatacgg gagggcttac |
| | catctggccc cagtgctgca |
| 5461 | atgataccgc gagacccacg ctcaccggct ccagatttat |
| | cagcaataaa ccagccagcc |
| 5521 | ggaagggccg agcgcagaag tggtcctgca actttatccg |
| | cctccatcca gtctattaat |
| 5581 | tgttgccggg aagctagagt aagtagttcg ccagttaata |
| | gtttgcgcaa cgttgttgcc |
| 5641 | attgctgcag gcatcgtggt gtcacgctcg tcgtttggta |
| | tggcttcatt cagctccggt |
| 5701 | tcccaacgat caaggcgagt tacatgatcc cccatgttgt |
| | gcaaaaaagc ggttagctcc |
| 5761 | ttcggtcctc cgatcgttgt cagaagtaag ttggccgcag |
| | tgttatcact catggttatg |
| 5821 | gcagcactgc ataattctct tactgtcatg ccatccgtaa |
| | gatgcttttc tgtgactggt |
| 5881 | gagtactcaa ccaagtcatt ctgagaatag tgtatgcggc |
| | gaccgagttg ctcttgcccg |
| 5941 | gcgtcaacac gggataatac cgcgccacat agcagaactt |
| | taaaagtgct catcattgga |
| 6001 | aaacgttctt cggggcgaaa actctcaagg atcttaccgc |
| | tgttgagatc cagttcgatg |
| 6061 | taacccactc gtgcacccaa ctgatcttca gcatctttta |
| | ctttcaccag cgtttctggg |
| 6121 | tgagcaaaaa caggaaggca aaatgccgca aaaaagggaa |
| | taagggcgac acggaaatgt |
| 6181 | tgaatactca tactcttcct ttttcaatat tattgaagca |
| | tttatcaggg ttattgtctc |
| 6241 | atgagcggat acatatttga atgtatttag aaaaataaac |
| | aaataggggt tccgcgcaca |
| 6301 | tttccccgaa aagtgccacc tgacgtctaa gaaaccatta |
| | ttatcatgac attaacctat |
| 6361 | aaaaataggc gtatcacgag gccctttcgt cttcaagaat |
| | taacaaaatt ctccagtctt |
| 6421 | cacatcggtt tgaaaggagg aagcggaaga atgaagtaag |
| | agggattttt gactccgaag |
| 6481 | taagtcttca aaaaatcaaa taaggagtgt caagaatgtt |
| | tgcaaaacga ttcaaaacct |
| 6541 | ctttactgcc gttattcgct ggatttttat tgctgtttca |
| | tttggttctg gcaggaccgg |
| 6601 | cggctgcgag tgctgaaacg gcgaacaaat cgaatgagct |
| | tacagcaccg tcgatcaaaa |
| 6661 | gcggaaccat tcttcatgca tggaattggt cgttcaatac |
| | gttaaaacac aatatgaagg |
| 6721 | atattcatga tgcaggatat acagccattc agacatctcc |
| | gattaaccaa gtaaaggaag |
| 6781 | ggaatcaagg agataaaagc atgtcgaact ggtactggct |
| | gtatcagccg acatcgtatc |
| 6841 | aaattggcaa ccgttactta ggtactgaac aagaatttaa |
| | agaaatgtgt gcagccgctg |
| 6901 | aagaatatgg cataaaggtc attgttgacg cggtcatcaa |
| | tcataccacc agtgattatg |
| 6961 | ccgcgatttc caatgaggtt aagagtattc caaactggac |
| | acatggaaac acacaaatta |
| 7021 | aaaactggtc tgatcgatag tacataatgg atttccttac |
| | gcgaaatacg ggcagacatg |
| 7081 | gcctgcccgg ttattaaaaa ataatgttgt ccttttaaat |
| | aagatctgat aaaatgtgaa |
| 7141 | ctaaatgtaa taattatagg cgatggagtt cgccataaac |
| | gctgcttagc taatgactcc |
| 7201 | taccagtatc actactggta ggagtctatt tttttgagca |
| | agctatttaa agaggtacta |
| 7261 | gtactgcagt ccggcaaaaa agggcaaggt gtcctagtaa |
| | ggtcgacagg aggactctct |
| 7321 | atgaaatttg gaaacttttt gcttacatac caacctcccc |
| | aattttctca aacagaggta |
| 7381 | atgaaacgtt tggttaaatt aggtcgcatc tctgaggagt |
| | gtggttttga taccgtatgg |
| 7441 | ttactggagc atcatttcac ggagtttggt ttgcttggta |
| | acccttatgt cgctgctgca |
| 7501 | tatttacttg gcgcgactaa aaaattgaat gtaggaactg |
| | ccgctattgt tcttcccaca |
| 7561 | gcccatccag tacgccaact tgaagatgtg aatttattgg |
| | atcaaatgtc aaaaggacga |
| 7621 | tttcggtttg gtatttgccg agggctttac aacaaggact |
| | ttcgcgtatt cggcacagat |
| 7681 | atgaataaca gtcgcgcctt agcggaatgc tggtacgggc |
| | tgataaagaa tggcatgaca |
| 7741 | gagggatata tggaagctga taatgaacat atcaagttcc |
| | ataaggtaaa agtaaacccc |
| 7801 | gcggcgtata gcagaggtgg cgcaccggtt tatgtggtgg |
| | ctgaatcagc ttcgacgact |
| 7861 | gagtgggctg ctcaatttgg cctaccgatg atattaagtt |
| | ggattataaa tactaacgaa |
| 7921 | aagaaagcac aacttgagct ttataatgaa gtggctcaag |
| | aatatgggca cgatattcat |
| 7981 | aatatcgacc attgcttatc atatataaca tctgtagatc |
| | atgactcaat taaagcgaaa |
| 8041 | gagatttgcc ggaaatttct ggggcattgg tatgattctt |
| | atgtgaatgc tacgactatt |
| 8101 | tttgatgatt cagaccaaac aagaggttat gatttcaata |
| | aagggcagtg gcgtgacttt |
| 8161 | gtattaaaag gacataaaga tactaatcgc cgtattgatt |
| | acagttacga aatcaatccc |
| 8221 | gtgggaacgc cgcaggaatg tattgacata attcaaaaag |
| | acattgatgc tacaggaata |
| 8281 | tcaaatattt gttgtggatt tgaagctaat ggaacagtag |
| | acgaaattat tgcttccatg |
| 8341 | aagctcttcc agtctgatgt catgccattt cttaaagaaa |
| | aacaacgttc gctattatat |
| 8401 | tagctaagga ggtaaagaaa tgaaatttgg attgttcttc |
| | cttaacttca tcaattcaac |
| 8461 | aactgttcaa gaacaaagta tagttcgcat gcaggaaata |
| | acggagtatg ttgataagtt |
| 8521 | gaattttgaa cagattttag tgtatgaaaa tcatttttca |
| | gataatggtg ttgtcggcgc |
| 8581 | tcctctgact gtttctggtt ttctgctcgg tttaacagag |
| | aaaattaaaa ttggttcatt |
| 8641 | aaatcacatc attacaactc atcatcctgt cgccatagcg |
| | gaggaagctt gcttattgga |
| 8701 | tcagttaagt gaagggagat ttattttagg gtttagtgat |
| | tgcgaaaaaa aagatgaaat |
| 8761 | gcattttttt aatcgcccgg ttgaatatca acagcaacta |
| | tttgaagagt gttatgaaat |
| 8821 | cattaacgat gctttaacaa caggctattg taatccagat |
| | aacgattttt atagcttccc |
| 8881 | taaaatatct gtaaatcccc atgcttatac gccaggcgga |
| | cctcggaaat atgtaacagc |
| 8941 | aaccagtcat catattgttg agtgggcggc caaaaaaggt |
| | attcctctca tctttaagtg |
| 9001 | ggatgattct aatgatgtta gatatgaata tgctgaaaga |
| | tataaagccg ttgcggataa |
| 9061 | atatgacgtt gacctatcag agatagacca tcagttaatg |
| | atattagtta actataacga |
| 9121 | agatagtaat aaagctaaac aagagacgcg tgcatttatt |
| | agtgattatg ttcttgaaat |
| 9181 | gcaccctaat gaaaatttcg aaaataaact tgaagaaata |
| | attgcagaaa acgctgtcgg |
| 9241 | aaattatacg gagtgtataa ctgcggctaa gttggcaatt |
| | gaaaagtgtg gtgcgaaaag |
| 9301 | tgtattgctg tcctttgaac caatgaatga tttgatgagc |
| | caaaaaaatg taatcaatat |
| 9361 | tgttgatgat aatattaaga agtaccacat ggaatatacc |
| | taataggtac caggaggaag |
| 9421 | gcaaatatga ctaaaaaaat ttcattcatt attaacggcc |
| | aggttgaaat ctttcccgaa |
| 9481 | agtgatgatt tagtgcaatc cattaatttt ggtgataata |
| | gtgtttacct gccaatattg |
| 9541 | aatgactctc atgtaaaaaa cattattgat tgtaatggaa |
| | ataacgaatt acggttgcat |
| 9601 | aacattgtca attttctcta tacggtaggg caaagatgga |
| | aaaatgaaga atactcaaga |
| 9661 | cgcaggacat acattcgtga cttaaaaaaa tatatgggat |
| | attcagaaga aatggctaag |
| 9721 | ctagaggcca attggatatc tatgatttta tgttctaaag |
| | gcggccttta tgatgttgta |
| 9781 | gaaaatgaac ttggttctcg ccatatcatg gatgaatggc |
| | tacctcagga tgaaagttat |
| 9841 | gttcgggctt ttccgaaagg taaatctgta catctgttgg |
| | caggtaatgt tccattatct |
| 9901 | gggatcatgt ctatattacg cgcaatttta actaagaatc |
| | agtgtattat aaaaacatcg |
| 9961 | tcaaccgatc cttttaccgc taatgcatta gcgttaagtt |
| | ttattgatgt agaccctaat |
| 10021 | catccgataa cgcgctcttt atctgttata tattggcccc |
| | accaaggtga tacatcactc |
| 10081 | gcaaaagaaa ttatgcgaca tgcggatgtt attgtcgctt |
| | ggggagggcc agatgcgatt |
| 10141 | aattgggcgg tagagcatgc gccatcttat gctgatgtga |
| | ttaaatttgg ttctaaaaag |
| 10201 | agtctttgca ttatcgataa tcctgttgat ttgacgtccg |
| | cagcgacagg tgcggctcat |
| 10261 | gatgtttgtt tttacgatca gcgagcttgt ttttctgccc |
| | aaaacatata ttacatggga |
| 10321 | aatcattatg aggaatttaa gttagcgttg atagaaaaac |
| | ttaatctata tgcgcatata |
| 10381 | ttaccgaatg ccaaaaaaga ttttgatgaa aaggcggcct |
| | attctttagt tcaaaaagaa |
| 10441 | agcttgtttg ctggattaaa agtagaggtg gatattcatc |
| | aacgttggat gattattgag |
| 10501 | tcaaatgcag gtgtggaatt taatcaacca cttggcagat |
| | gtgtgtacct tcatcacgtc |
| 10561 | gataatattg agcaaatatt gccttatgtt caaaaaaata |
| | agacgcaaac catatctatt |
| 10621 | tttccttggg agtcatcatt taaatatcga gatgcgttag |
| | cattaaaagg tgcggaaagg |
| 10681 | attgtagaag caggaatgaa taacatattt cgagttggtg |
| | gatctcatga cggaatgaga |
| 10741 | ccgttgcaac gattagtgac atatatttct catgaaaggc |
| | catctaacta tacggctaag |
| 10801 | gatgttgcgg ttgaaataga acagactcga ttcctggaag |
| | aagataagtt ccttgtattt |
| 10861 | gtcccataat ggaggtaaaa gtatggaaaa tgaatcaaaa |
| | tataaaacca tcgaccacgt |
| 10921 | tatttgtgtt gaaggaaata aaaaaattca tgtttgggaa |
| | acgctgccag aagaaaacag |
| 10981 | cccaaagaga aagaatgcca ttattattgc gtctggtttt |
| | gcccgcagga tggatcattt |
| 11041 | tgctggtctg gcggaatatt tatcgcggaa tggatttcat |
| | gtgatccgct atgattcgct |
| 11101 | tcaccacgtt ggattgagtt cagggacaat tgatgaattt |
| | acaatgtcta taggaaagca |
| 11161 | gagcttgtta gcagtggttg attggttaac tacacgaaaa |
| | ataaataact tcggtatgtt |
| 11221 | ggcttcaagc ttatctgcgc ggatagctta tgcaagccta |
| | tctgaaatca atgcttcgtt |
| 11281 | tttaatcacc gcagtcggtg ttgttaactt aagatattct |
| | cttgaaagag ctttagggtt |
| 11341 | tgattatctc agtctaccca ttaatgaatt gccggataat |
| | cttgattttg aaggccataa |
| 11401 | attgggtgct gaagtctttg cgagagattg tcttgatttt |
| | ggttgggaag atttagcttc |
| 11461 | tacaattaat aacatgatgt atcttgatat accgtttatt |
| | gcttttactg caaataacga |
| 11521 | taattgggtc aagcaagatg aagttatcac attgttatca |
| | aatattcgta gtaatcgatg |
| 11581 | caagatatat tctttgttag gaagttcgca tgacttgagt |
| | gaaaatttag tggtcctgcg |
| 11641 | caatttttat caatcggtta cgaaagccgc tatcgcgatg |
| | gataatgatc atctggatat |
| 11701 | tgatgttgat attactgaac cgtcatttga acatttaact |
| | attgcgacag tcaatgaacg |
| 11761 | ccgaatgaga attgagattg aaaatcaagc aatttctctg |
| | tcttaaagaa tcctgaggag |
| 11821 | gaaaacaggt atgacttcat atgttgataa acaagaaatt |
| | acagcaagct cagaaattga |
| 11881 | tgatttgatt ttttcgagcg atccattagt gtggtcttac |
| | gacgagcagg aaaaaatcag |
| 11941 | aaagaaactt gtgcttgatg catttcgtaa tcattataaa |
| | cattgtcgag aatatcgtca |
| 12001 | ctactgtcag gcacacaaag tagatgacaa tattacggaa |
| | attgatgaca tacctgtatt |
| 12061 | cccaacatcg gtttttaagt ttactcgctt attaacttct |
| | caggaaaacg agattgaaag |
| 12121 | ttggtttacc agtagcggca cgaatggttt aaaaagtcag |
| | gtggcgcgtg acagattaag |
| 12181 | tattgagaga ctcttaggct ctgtgagtta tggcatgaaa |
| | tatgttggta gttggtttga |
| 12241 | tcatcaaata gaattagtca atttgggacc agatagattt |
| | aatgctcata atatttggtt |
| 12301 | taaatatgtt atgagtttgg tggaattgtt atatcctacg |
| | acatttaccg taacagaaga |
| 12361 | acgaatagat tttgttaaaa cattgaatag tcttgaacga |
| | ataaaaaatc aagggaaaga |
| 12421 | tctttgtctt attggttcgc catactttat ttatttactc |
| | tgccattata tgaaagataa |
| 12481 | aaaaatctca ttttctggag ataaaagcct ttatatcata |
| | accggaggcg gctggaaaag |
| 12541 | ttacgaaaaa gaatctctga aacgtgatga tttcaatcat |
| | cttttatttg atactttcaa |
| 12601 | tctcagtgat attagtcaga tccgagatat atttaatcaa |
| | gttgaactca acacttgttt |
| 12661 | ctttgaggat gaaatgcagc gtaaacatgt tccgccgtgg |
| | gtatatgcgc gagcgcttga |
| 12721 | tcctgaaacg ttgaaacctg tacctgatgg aacgccgggg |
| | ttgatgagtt atatggatgc |
| 12781 | gtcagcaacc agttatccag catttattgt taccgatgat |
| | gtcgggataa ttagcagaga |
| 12841 | atatggtaag tatcccggcg tgctcgttga aattttacgt |
| | cgcgtcaata cgaggacgca |
| 12901 | gaaagggtgt gctttaagct taaccgaagc gtttgatagt |
| | tga. |

In embodiments, the riboswitch is operatively linked to a reporter-encoding sequence such that the riboswitch controls activity, e.g., translation, of the reporter-encoding sequence reporter-encoding sequence. In embodiments, nucleic acid (e.g., DNA) encoding the riboswitch is integrated into a bacterial genome, e.g., in the amyE or crcB locus. In embodiments, the spore-forming bacterium is of the phylum Firmicute, e.g., of the genus Bacillus or Clostridium. In embodiments, the bacterium further comprises a crcB deficiency, e.g., deletion. A crcB disruption can be made at the same time as integration of the biosensor gene, or can be made in a separate step. In some embodiments, the endogenous crcB riboswitch sequence is preserved and a reporter gene is inserted in place of the crcB coding sequence, thus adding a reporter and disrupting the crcB locus with one integration event. In embodiments, the bacterium is B. subtilis, e.g., B. subtilis PY97, which optionally comprises a trpC mutation in 168 (e.g., requiring tryptophan supplementation in minimal medium).

In embodiments, the riboswitch binds fluoride ion. In embodiments, the riboswitch is a crcB, 78 Psy, eriC, or eriC^{F} riboswitch. In embodiments, the riboswitch is a Methylobacterium extorquens DM4 riboswitch, e.g., a fluoride riboswitch. Exemplary suitable riboswitches are described in Baker et al., “Widespread Genetic Switches and Toxicity Resistance Proteins for Fluoride” Science. 2012 Jan. 13; 335(6065): 233-235, which is herein incorporated by reference in its entirety.

Numerous reporters can be used, e.g., in conjunction with a riboswitch, including fluorescent reporter proteins (e.g., GFP, EGFP, RFP, mRFPmars, YFP, iYFP, mYPET, CFP, BFP, EBFP, Azurite, mKalama1, mCherry, mOrange, TagBFP, mTurquoise, Cerulean, ECFP, CyPet, mTurquoise2, Citrine, Venus, YPet, sapphire GFP, sgBFP, sgGFP, dsRed, eqFP611, Dronpa, TagRFPs, KFP, EosFP, Dendra, IrisFP, FbFPs, smURFP), luminescent reporter proteins (e.g., aequorin, luciferase, one or more (e.g., all) components of the lux operon, e.g., LuxCDABE), or colorimetric reporter proteins such as lacZ.

In embodiments where the reporter protein is fluorescent, the device will generally comprise a source of an excitation wavelength. In embodiments where the reporter protein is luminescent, a source of excitation wavelength is generally not needed.

The riboswitch can also be operatively linked to an intermediate regulatory protein, wherein the intermediate regulatory protein modulates levels of a reporter protein. The reporter-encoding sequence need not be operatively linked to the riboswitch in such embodiments. This design adds an additional step between sensing analyte and outputting an optical characteristic. As an example, the riboswitch drives expression of T7 RNA polymerase, and expression of the reporter protein (e.g., a fluorescent protein) is driven by a T7 promoter. Thus, upon binding of the analyte to the riboswitch, T7 RNA polymerase is produced, which leads to expression of the reporter protein. In embodiments, the riboswitch, T7 pol gene, T7 promoter, and reporter gene are in the same nucleic acid, and in some embodiments, the riboswitch and T7 pol gene are on a first nucleic acid and the T7 promoter and reporter gene are on a second nucleic acid. When all four components are on one nucleic acid, a terminator may be introduced (e.g., upstream of the T7 promoter) to prevent unintended transcription of the reporter gene from a promoter other than the T7 promoter. Alternatively or in combination, when all four components are on the same nucleic acid, the two coding regions may be placed in opposite orientations to prevent unintended transcription of the reporter gene from a promoter other than the T7 promoter.

The biological component (e.g., a biotic component, e.g., yeast, e.g., S. cerevisiae) can comprise an olfactory sensor, e.g., Olfr226, Olfr2, or MOR226-1, that modulates levels of a reporter protein, e.g., a reporter protein described herein, e.g., a fluorescent, luminescent, or colorimetric reporter. In embodiments, the strain (e.g., WIF-1 or WIF-1α) comprises other components of the olfactory signaling pathway). In embodiments, the biological component is configured to sense DNT or TNT. An exemplary DNT biosensor is described in Radhika et al., “Chemical sensing of DNT by engineered olfactory yeast strain” Nature Chemical Biologic 3(6) 325-330 (June 2007), which is herein incorporated by reference in its entirety.

The biological component (e.g., a biotic component, e.g., bacteria) can comprise a sensor for a reactive oxygen species, e.g., H₂O₂. The bacterium can comprise an H₂O₂ sensing protein such as bacterial OxyR, that modulates levels of a reporter protein, e.g., a reporter protein described herein, e.g., a fluorescent, luminescent, or colorimetric reporter. An exemplary H₂O₂ biosensor is described in Ermakova et al., “Red fluorescent genetically encoded indicator for intracellular hydrogen peroxide” Nat Commun. 2014 Oct. 21; 5:5222, which is herein incorporated by reference in its entirety.

The biological component (e.g., a biotic component, e.g., bacteria) can comprise a sensor for radiation, e.g., ionizing radiation, e.g., gamma irradiation. The bacterium can comprise a radiation sensor such as a stress promoter, e.g., a promoter for recA, grpE, or katG. The promoter can drive expression of a reporter protein, e.g., a reporter protein described herein, e.g., a fluorescent, luminescent, or colorimetric reporter. An exemplary radiation biosensor is described in Min et al., “Detection of radiation effects using recombinant bioluminescent Escherichia coli strains” Radiat Environ Biophys (2000) 39:41-45, which is herein incorporated by reference in its entirety.

The biological component (e.g., a biotic component, e.g., bacteria) can comprise a sensor for a pollutant, e.g., m-xylene, toluene, or 3-methylbenzylalcohol (3MBA). The bacterium can comprise a pollutant sensor such as a transcription factor that binds and is modulated by the pollutant, e.g., XylR. The activated transcription factor can drive expression of a reporter protein, e.g., a reporter protein described herein, e.g., a fluorescent, luminescent, or colorimetric reporter. An exemplary m-xylene biosensor is described in de las Heras et al., “Increasing Signal Specificity of the TOL Network of Pseudomonas putida mt-2 by Rewiring the Connectivity of the Master Regulator XylR” PLOS Genetics, 8:10 e1002963 (October 2012), which is herein incorporated by reference in its entirety.

The biological component (e.g., a biotic component, e.g., bacteria) can comprise a sensor for a heavy metal, e.g., uranium, cadmium, chromate, or dichromate. The bacterium can comprise a heavy metal sensor such as a P_{urcA} promoter. The promoter may be a promoter of a heavy metal responsive protein of a Caulobacler species, e.g. Caulobacler crescenlus CC3302, CC1777; CC3500; CC1532; and CC3291, or a homolog or variant thereof. The promoter can drive expression of a reporter protein, e.g., a reporter protein described herein, e.g., a fluorescent, luminescent, or colorimetric reporter. Exemplary heavy metal sensors are described in U.S. Pat. No. 8,697,388, which is herein incorporated by reference in its entirety.

The biological component (e.g., a biotic component, e.g., bacteria) can comprise a sensor for a heavy metal, e.g., mercury. The bacterium can comprise a heavy metal sensor such as a mercury-inducible promoter e.g., the mer promoter from transposon Tn21. The promoter can drive expression of a reporter protein, e.g., a reporter protein described herein, e.g., a fluorescent, luminescent, or colorimetric reporter. An exemplary mercury biosensor is described in Virta et al., “A Luminescence-Based Mercury Biosensors” Anal. Chem. 67, 667-669 (1995), which is herein incorporated by reference in its entirety.

The biological component (e.g., a biotic component, e.g., bacteria) can comprise a sensor for a heavy metal, e.g., copper or zinc. The bacterium can comprise a heavy metal sensor that drives expression of a reporter protein, e.g., a reporter protein described herein, e.g., a fluorescent, luminescent, or colorimetric reporter. Exemplary copper and zinc biosensors are described in Chaudri et al., “Response of a Rhizobium-based luminescence biosensor to Zn and Cu in soil solutions from sewage sludge treated soils” Volume 32, Issue 3, March 2000, Pages 383-388, which is herein incorporated by reference in its entirety.

According to some embodiments, the biological component is abiotic. For example, in some embodiments, the biological component comprises a protein. In accordance with certain aspects, the proteins may be enzymes. In embodiments, the enzyme catalyzes a chemical reaction yielding a detectable reaction product, e.g., a molecule that absorbs and/or emits light. In embodiments, the enzyme produces light.

In accordance with one embodiment, the abiotic may be a nucleic acid enzyme such as a ribozyme, DNAzyme, an RNA/DNA hybrid enzyme, or a peptide nucleic acid (PNA)zyme. Nucleic acid enzymes are nucleic acid molecules that catalyze a chemical reaction. In some embodiments, the abiotic biological component is a DNA molecule, such as a deoxyribozyme (DNAzyme or DNA enzyme). Using abiotics as the biological component presents a cell-free method for detecting target analytes.

In accordance with some embodiments, the nucleic acid enzyme, such as a DNAzyme, may be lyophilized or otherwise subjected to a drying process. The drying process may include placing the nucleic acid enzyme in a solution and then performing a freeze-drying process. In some embodiments, the solution may be an aqueous solution. The solution may contain one or more stabilizers such as a surfactant. Lyophilization can be performed, e.g., as described above.

The biological component may be an engineered material, including organisms, biomolecules, and/or nucleic acid enzymes for detection of analytes, signal transduction, and signal readout. As used herein, engineered material refers to any material that has been created and/or altered by man and is therefore a non-naturally-occurring material. According to one or more embodiments, the engineered biological component may be tuned to react in any one or more different ways in response to sensing a target analyte. For example, the engineered biological component may be engineered to fluoresce or emit light at certain wavelengths or produce an electrical output. In accordance with certain embodiments, the engineered material may be at least one of bacteria, enzymes, including nucleic acid enzymes, antibodies, nucleic acid, cells, tissues, and organelles.

In general, engineering the biological component may allow the biological component to be tuned to a particular target analyte, such as one or more chemicals. For example, according to some embodiments, the engineered material may be tuned to sense one or more gas analytes, such as chlorine, carbon dioxide, carbon monoxide, mercury, ethylene oxide, sulfur dioxide, hydrogen sulfide, etc. In some embodiments, the engineered material may be tuned to sense an analyte, where the analyte is a chemical presented as a vapor or an aerosol.

According to other embodiments, the engineered material may be tuned to sense one or more chemicals (which may also be in the form of a gas, vapor, or aerosol), that are associated with explosives, such as trinitrotoluene (TNT), 2,4,-dinitrotoluene (DNT) which is released as an impurity by TNT, nitroglycerine (NG), ethylene glycol dinitrate (EGNG), ammonium nitrate (NH₄NO₃), cyclohexanone, benzoquinone, 2-ethyl hexanol, Triacetin, diphenylamine (DPA), ethylene glycol dinitrate (EGDN), and dimethyl dinitrobutane (DMNB).

In accordance with other embodiments, the engineered material may be tuned to a radioactive target analyte, and may thus be configured to detect a certain level of gamma radiation from one or more radioisotopes. Non-limiting examples include radioisotopes of uranium, plutonium, cesium, cobalt, iridium, strontium, and radium. In other embodiments, the engineered material may be tuned to one or more contaminants, such as arsenic or selenium. In other embodiments, the engineered material may be tuned to one or more allergens, such as foodstuffs, pollen, dust, mold, animal dander, latex, and drugs. In some embodiments, the engineered material may be tuned to one or more pollutants, such as particulate matter, carbon monoxide, nitrogen oxides, sulfur dioxide, and lead. In some embodiments, the engineered material may be tuned to one or more agents of chemical and/or biological warfare, such as anthrax. In some embodiments, the engineered material may be tuned to one or more agents that provoke a physiological response, such as fragrances or other odors, or pheromones.

In accordance with some embodiments, the biological component may be genetically engineered. As used herein, genetically engineered or otherwise genetically modified material refers to any material that has been altered using genetic engineering techniques, e.g., is a daughter cell of a cell that was altered using a genetic engineering technique or is a nucleic acid copy of a nucleic acid that was altered using a genetic engineering technique. For example, one or more desired genetic traits may be artificially introduced using a genetic manipulation technique. In some embodiments, the genetically engineered material includes one or more living organisms that have been genetically engineered so as to change their genetic composition.

According to one embodiment, the genetically engineered material comprises bacteria. The bacteria may be any type of bacteria, for example, gram positive bacteria or gram negative bacteria. The bacteria may be aerobic or anaerobic. The bacteria may be of any morphology. For example, the bacteria may be spherical or rod-shaped. The bacteria may be of any metabolic type. For example, the bacteria may be heterotrophic or autotrophic. The bacteria can be, e.g., of the phylum Firmicute, e.g., of the genus Bacillus or Clostridium. According to some embodiments, the bacteria comprise Bacillus subtilis. In certain embodiments, more than one type of bacteria may be used to coordinate sensing in a multiplexed way.

According to other aspects, the genetically engineering material may include an engineered bacterium that is tuned to react in any one or more of a number of different ways in response to sensing a target analyte. For example, the bacteria may be genetically engineered to fluoresce or to produce an electrical output. According to some embodiments, the genetically engineered bacteria may be tuned to produce certain small molecules (e.g., autoinducers for quorum sensing), nucleotides (e.g., cyclic di-GMP to activate riboswitches to control gene expression), or biomolecules (e.g., signaling peptides that facilitate the SEC secretion system in gram negatives) as indirect response elements.

In accordance with some embodiments, the bacteria may be genetically engineered by a process of introducing a foreign DNA, for example, a plasmid, into the bacteria. This process comprises fully integrating foreign DNA into the chromosomes of the bacteria, and thus changes the genetic make-up of the bacteria. In embodiments, this can be accomplished via homologous recombination, in which the gene sequence that is to be integrated into the chromosome contains a region that is homologous to the bacterial chromosome. The integration occurs via endogenous recombination machinery such as that of E. coli or mediated by a phage specific integrase. As will be appreciated by those of skill in the art, according to some embodiments, homologous recombination involves the breakage of a double-strand of DNA. Sections of DNA around the 5′ ends of the break are cut away in a process called resection. In the strand invasion step that follows, an overhanging 3′ end of the broken DNA molecule then “invades” a similar or identical DNA molecule that is not broken. After strand invasion, the sequence of events may then follow either of two main pathways: the double-strand break repair pathway, or the synthesis-dependent strand annealing pathway. Integration can also be performed as site-specific integration or random integration.

In accordance with some embodiments, the genetically engineered material may be prepared so as to selectively detect a target or analyte. In some embodiments, a genetically engineered material may be prepared to be sensitive towards chlorine. For example, a gene from E. coli that has been identified to specifically encode a regulatory protein that may be introduced into another bacterium, such as B. subtilis. The regulator protein is capable of specifically binding chlorine. While the unbound form of the regulatory protein binds to a corresponding promoter and represses expression, the chlorine-bound form of the regulatory protein has reduced affinity for the promoter, and dissociates, allowing transcription of a gene controlled by the promoter. This promoter-regulator system may be paired with reporter genes that react to the presence of a target analyte. For example, the reporter genes may express fluorescence in the presence of the target analyte, such as chlorine.

In accordance with some embodiments, the genetically engineered material is prepared through a directed evolution process, which favors the organism that expresses the best response to the target analyte. In some embodiments, the bacteria are exposed to the target analyte and under conditions that favor growth or survival of bacteria that can detect the target analyte. For instance, a candidate sensor macromolecule in the bacteria can control the activity or level of a factor that promotes growth or survival, such as an antibiotic resistance gene. The response of the detector system may be reprogrammed to have a particular response, similar to the building of logic circuits.

In accordance with some embodiments, the genetically engineered material may respond to the presence of a general class of analytes. For example, the genetically engineered material that has been tuned to respond to the presence of arsenic may also respond to a class of similar metals. In this example, the genetically engineered material may be engineered with additional specificity, or may be used in combination with a second biological component that provides additional specificity. The additional specificity may be obtained through, for example, logic circuits or custom engineering of genetic and/or protein elements.

According to various aspects, bacteria may be selected to function as the biological component used in the sensor system based on any one or more of a number of different factors. For instance, bacteria may be selected based on its ability to form spores or otherwise tolerate dessication. For instance, bacteria may be selected based on its conduciveness to genetic engineering or manipulation. For example, the bacteria may be selected based on its capability of outputting electrical signals. According to another example, the bacteria may be selected based on its reactivity to one or more target analytes. For instance, the bacteria may be selected based on its reactivity to chlorine, carbon dioxide, and/or heavy metals. According to a further example, the bacteria may be selected based on its fluorescence or bioluminescence. According to some embodiments, the bacteria may be selected based on its ability to sense a target analyte. For example, once the bacteria sense a target, they may produce a characteristic protein, such as an enzyme, that is detectable. According to another aspect, the bacteria may increase the production of another output molecule in reaction to a target analyte, which can then be detected. In certain embodiments, these bacteria are genetically engineered to react using one or more of the modes discussed herein.

According to at least one embodiment, a nucleic acid enzyme may be engineered to exhibit one or more characteristics in the presence of a target analyte. The target analyte may be any of the target analytes recited above in reference to the biotic biological component.

In accordance with some embodiments, functional DNA molecules such as deoxyribozymes or DNAzymes may be isolated for specific targets through an in vitro selection method. The nucleic acid may comprise DNA (e.g., may be a DNAzyme), RNA (e.g., may be a ribozyme), and/or may comprise chemically modified nucleotides.

In embodiments, a biological component comprises two nucleic acid strands which are together in the absence of an analyte and separate in the presence of the analyte, wherein the nucleic acid strands have different optical properties when they are together versus separate, thereby producing an optical signal indicating the presence, absence, or level of the analyte.

For example, in some embodiments, the nucleic acid enzyme (e.g., DNAzyme) comprises a first nucleic acid strand and a second nucleic acid strand which have partial complementarity to each other. The first nucleic acid strand may comprise a fluorophore and the second nucleic acid strand may comprise a quencher, or vice versa, such that when the first and second nucleic acid strands hybridize, the fluorophore and the quencher are in close proximity and fluorescence is blocked. The nucleic acid enzyme may further have a cleavage site (e.g., on the first stand), where the cleavage site is cleaved when an analyte (e.g., uranium or lead) is present. Cleavage converts the first strand into two shorter strands, one or each having a weaker affinity for the second strand. When the fluorophore-containing strand dissociates from the quencher-containing strand, fluorescence increases. Exemplary DNAzymes for detecting uranium or lead are described in Liu et al “A catalytic beacon sensor for uranium with parts-per-trillion sensitivity and millionfold selectivity” PNAS 104:7 2056-2061 (2007), which is incorporated herein by reference in its entirety.

In some embodiments, the nucleic acid enzyme is a Multicomponent nucleic acid enzyme (MNAzyme), e.g., as described in US Pat. App. Pub. No. 2012/0178078, which is incorporated herein by reference in its entirety.

In some embodiments, the biosensor comprises a first nucleic acid strand having a first fluorophore (e.g., a quantum dot, e.g., a CdTe quantum dot), and a second nucleic acid having a second fluorophore (e.g., TAMRA). The first and second nucleic acids can have at least partial complementarity, allowing them to hybridize to each other. The first and second fluorophores can be capable of fluorescent resonance energy transfer (FRET) such that their fluorescent properties are different when they are in proximity versus distant. The presence of a DNA analyte (e.g., DNA from a pathogen, e.g., H. pylori) can compete for binding, causing the two strands to separate, which in turn causes loss of FRET. A sensor of this type is described in Shanehsaz et al., “Detection of Helicobacter pylori with a nanobiosensor based on fluorescence resonance energy transfer using CdTe quantum dots” Microchimica Acta, February 2013, Volume 180, Issue 3, pp 195-202, which is herein incorporated by reference in its entirety.

In some embodiments, the biosensor comprises an enzyme and a non-fluorescent substrate that is converted to a fluorescent form in the presence of an analyte. For instance, in embodiments the biosensor comprises horseradish peroxidase (HRP) and N-acetyl-3,7-dihydroxyphenoxazine (Amplex Red) for the detection of hydrogen peroxide. In embodiments, the Amplex Red is enzymatically converted to the fluorescent molecule resorufin in the presence of hydrogen peroxide, such that presence of the analyte leads to an increase in fluorescence. A biosensor of this type is described, e.g., in Zhou et al., “A Stable Nonfluorescent Derivative of Resorufin for the Fluorometric Determination of Trace Hydrogen Peroxide: Applications in Detecting the Activity of Phagocyte NADPH Oxidase and Other Oxidases” Analytical Biochemistry 253, 162-168 (1997), which is herein incorporated by reference in its entirety. In embodiments, the biosensor comprises an aptamer (a nucleic acid able to bind another molecule with high affinity). In embodiments, during manufacturing, a baseline amount of an analyte (e.g., TNT) is coated on a surface of the device. When the device is used, an aptamer conjugated to a label having an optical characteristic (e.g., a fluorescent moiety) is added to the surface. In the absence of analyte in the environment, the aptamer binds the surface, resulting in an optical signal (e.g., fluorescence) on the surface. When analyte is present in the environment, it competes away aptamer from the surface, resulting in reduced levels of the optical signal on the surface. A biosensor of this type, for detecting TNT, is described in Ehrentreich-Förster et al., “Biosensor-based on-site explosives detection using aptamers as recognition elements” Anal Bioanal Chem (2008) 391:1793-1800, which is herein incorporated by reference in its entirety.

In accordance with one embodiment, one or more biological components may be configured to detect uranyl and fluoride ions. Uranium hexafluoride (UF₆) is the chemical form of uranium that is used during the uranium enrichment process. Although UF₆ does not react with other components in the air, such as oxygen, nitrogen, carbon dioxide, or dry air, it reacts with water vapor to form hydrogen fluoride (HF) and uranyl fluoride (UO₂F₂). The presence of both uranyl and fluoride ions is therefore an indicator of uranium enrichment. In some embodiments, one biological component may be engineered to detect uranyl ions and another biological component may be engineered to detect fluoride ions. In one embodiment, bacteria such as Bacillus subtilis is configured to detect and fluoresce in the presence of fluoride ions and a nucleic acid enzyme, such as a DNAzyme, is configured to detect uranyl ions and fluoresce when the ions are present. According to other embodiments, a single biological component may be engineered to detect both uranyl and fluoride ions.

According to some embodiments, the biological component may be configured to detect low concentrations of the target analyte. For example, in some embodiments, the biological component is configured to detect the target analyte at concentration levels down to a level of about 1 ppm. In one embodiment, the biological composition is an engineered bacteria that detects fluoride ions at a concentration level of 1 ppm. According to other embodiments, the biological component is configured to detect the target analyte at concentrations of about 1 ppb. In one embodiment, the biological component is an engineered DNAzyme that is configured to detect uranyl ions at a concentration level of about 1 ppb.

In accordance with certain embodiments, instead of a biological component, the sensor may comprise a non-biological component. In accordance with one or more aspects, the lifetime of non-biological sensors can advantageously be extended using the devices described herein, e.g., when (similar to a biological component) the non-biological component is activated when placed in solution, but is subject to degradation or inactivation when in solution for extended periods of time, i.e., the length of time the sensor is positioned at a monitoring site before activation.

In some embodiments, the sensor comprises a nanoparticle fluorophore/quencher system. For example, the sensor can comprise a fluorophore, e.g. quantum dots, e.g., GSH-capped CdTe QDs. The sensor can further comprise a quencher, e.g., a nanoparticle, e.g., a gold nanoparticle. In embodiments, the gold nanoparticles quench fluorescence via the inner filter effect. A target analyte can adsorb to the surface of the nanoparticles, blocking their quenching activity, leading to an optical change, e.g., a color change and/or increase in fluorescence. In embodiments, the analyte is a small molecule, e.g., a herbicide, e.g., cyanazine. A sensor of this type is described in Dong et al., “Highly sensitive colorimetric and fluorescent sensor for cyanazine based on the inner filter effect of gold nanoparticles” Journal of Nanoparticle Research June 2016, 18:164, which is herein incorporated by reference in its entirety.

### Biotransducer

According to one or more embodiments, the biological component may include a biotransducer. The biotransducer may function as a recognition-transduction component that converts a biochemical signal to an electronic or optical signal. A physiochemical transducer may also be included in the biotransducer that functions to transform a signal resulting from the interaction of the target analyte with the biological element into another signal that can be more easily measured and quantified. For instance, the biotransducer may convert a biochemical signal to an electronic or optical signal. According to various aspects, the biotransducer comprises two intimately coupled parts, a bio-recognition layer, and a physicochemical transducer.

In accordance with some embodiments, the bio-recognition layer may comprise any component that is bioselective. In some embodiments, the bio-recognition layer may comprise an enzyme or another binding protein, for example, an antibody. In other embodiments, the bio-recognition layer may comprise one or more of oligonucleotide sequences, sub-cellular fragments such as organelles and receptor carrying fragments, single whole cells, small numbers of cells on synthetic scaffolds, or thin slices of animal or plant tissues.

According to certain embodiments, the physiochemical transducer transforms a signal resulting from the interaction of the target analyte with the biological element into another signal that can be more easily measured and quantified. According to some embodiments, the physicochemical transducer may be in close proximity to the recognition layer. As a result of the presence and biochemical action of the analyte, a physicochemical change is produced within the biorecognition layer. The physicochemical change is measured by the physicochemical transducer, which produces a signal, e.g., such as a luminescent or fluorescent signal, that is proportionate to the concentration of the analyte. The physicochemical transducer may be electrochemical, optical, electronic, gravimetric, pyroelectric, or piezoelectric. The physicochemical transducer may be selected based on the signal received from the engineered material. For example, the physicochemical transducer may be an electrode if the engineered material emits an electroactive material. In another example, the physicochemical transducer may be a semiconducting pH electrode if the signal from the engineered material results in a change in pH. The physicochemical transducer may be a thermistor if the engineered material emits heat. In another example, the physicochemical transducer may be a photodetector if the engineered material emits light, or changes color. For instance, bacteria may fluoresce or change color in the presence of certain analytes. According to yet another example, the physicochemical transducer may be a piezoelectric medium if the signal from the engineered material causes a change in mass.

Referring to FIG. 6, an example of a sensor system 600 is shown that illustrates one example of the functionality of a biotransducer. The sensor system 600 comprises an abiotic or biotic element 680 and a biotransducer 690. The abiotic or biotic element 680 may comprise an engineered material, such as biologically sensitive bacteria. The abiotic or biotic element 680 functions to interact or otherwise detect a target analyte 685 and produces a first signal 694 in response. The first signal 694 is transmitted to the biotransducer 690. The biotransducer 690 may comprise a biorecognition layer 691 and a physicochemical transducer 692. The biotransducer 690 transforms the first signal 694 resulting from the interaction of the target analyte 685 with the abiotic or biotic element 680 into a second signal 696 that can be more easily measured and quantified. A reader device 640 comprises signal processors that process the second signal 696 and converts the second signal 696 into a result 697.

According to some embodiments, the biotransducer is an optical biotransducer. An optical biotransducer uses photons in order to collect information about an analyte. Optical biotransducers are highly selective, small in size, and cost effective. The detection mechanism of an optical biotransducer may depend upon the enzyme system that converts analytes into products which are either oxidized or reduced at the working electrode.

In some embodiments, the biotransducer is a field-effect transistor (FET)-based electronic biotransducer. The FET may directly translate the interactions between the analytes and the FET surface into readable electrical signals. In an FET, current flows along the channel which is connected to the source and the drain. The channel conductance between the source and the drain is switched on and off by a gate electrode that is capacitively coupled through a thin dielectric layer. In FET-based biosensors, the channel may be in direct contact with the environment, giving better control over the surface charge.

According to some embodiments, the biotransducer may be a wireless device. In some embodiments, the wireless device emanates a first signal having a first frequency and enables the wireless sensor to emanate a second signal having a second frequency upon attraction of a specific analyte to the wireless sensor.

In accordance with at least one embodiment, the biotransducer may transmit a signal to a detection system. For instance, the biotransducer may transmit an electromagnetic signal to a detection system. The detection system may comprise, for example, a radio frequency identification (RFID) tag and a reader for receiving and processing signals from the RFID tag. The reader may be configured to measure a signal from the RFID tag and at least one additional parameter from which a signal is derived. The RFID tag may be passive, semi-passive, or active. The passive RFID tag does not need a power source for operation, while the semi-passive and active RFID tags rely on the use of onboard power for their operation. In some embodiments, the detection system comprises amplifiers, filters, or multiplexers. In some embodiments, the detection system comprises analog-to-digital converters, linearizers, or compressors.

According to a further aspect, the engineered biological component, such as bacteria, may be “programmed” or otherwise configured to include genetically encoded digital amplifying genetic switches that function to detect one or more target analytes and, in response, perform signal digitization and/or amplification. For instance, bacteria may comprise digital amplifying switches that are capable of signal amplification, and in some cases, can also perform data storage and record transient signals. Thus, not only can the bacteria detect and amplify a signal associated with a target analyte, but the bacteria may also be capable of storing data for a period of time. This is useful in situations where there is a delay in time in retrieving or otherwise obtaining information from the bacteria.

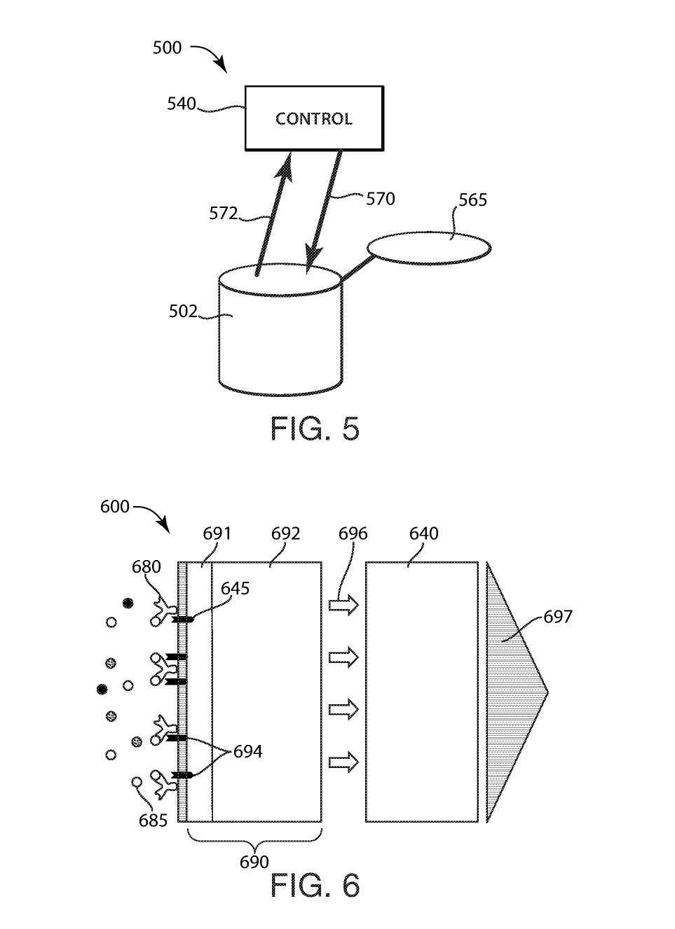

Referring to FIG. 5, a pictorial diagram of an example process 500 in accordance with one or more of the methods and systems disclosed herein is shown. According to the process 500 shown in FIG. 5, an activation signal 570 is sent to by a controller 540 to a sensor system 502. The sensor system 502 may be configured with similar features and elements as described previously with respect to FIGS. 2A and 2B. The activation signal 570 may be initiated by the controller based on any one of a number of different criteria, such as a predetermined time period, detection of a sample within the assay chamber, or based on external factors, such as suspected releases of the target analyte within the vicinity of the sensor system 502. In some instances, the activation signal 570 may prompt the sensor system 502 to obtain a sample 565. According to some embodiments, the sensor system 502 detects the presence of a sample 565 in the assay chamber 502 and therefore the activation signal 570 may be produced by a controller integrated within the sensor system 502 itself.

Once the sample 565 is obtained by the sensor system 502, a process for determining the presence or absence of the target analyte is conducted as described above in reference to the process 300 of FIGS. 3A and 3B. An output signal 572 may be transmitted by the sensor system 502 after process 300 is performed. The output signal 572 can contain data such as raw analysis data, (such as fluorescence measurements) that may be later interpreted by a user, or may contain more sophisticated analysis data performed by the analysis device, such as a concentration calculation of a target analyte.

### EXAMPLES

The function and advantages of the described and other embodiments will be more fully understood from the following examples. The examples are intended to be illustrative in nature and are not to be considered as limiting the scope of the systems and methods described herein.

### Example 1—Thermal Stability of Lyophilized Bacteria

Wild-type bacteria were lyophilized, subjected to different temperatures for a predetermined period of time, and then reconstituted in culture media, where their growth rates were measured. Two different strains of Bacillus subtilis (B. subtilis PY79 and B. subtilis AG174) spores were freeze-dried from vegetative cells. The bacteria were grown in a medium that promoted sporulation until starvation (1-7 days), after which the vegetative cells and spores were collected via a centrifugation or filtration process. The vegetative cells were destroyed by incubation with lysozyme at 37° C. Cellular debris was washed away with distilled water. The spores were then frozen and subjected to a lyophilization process.

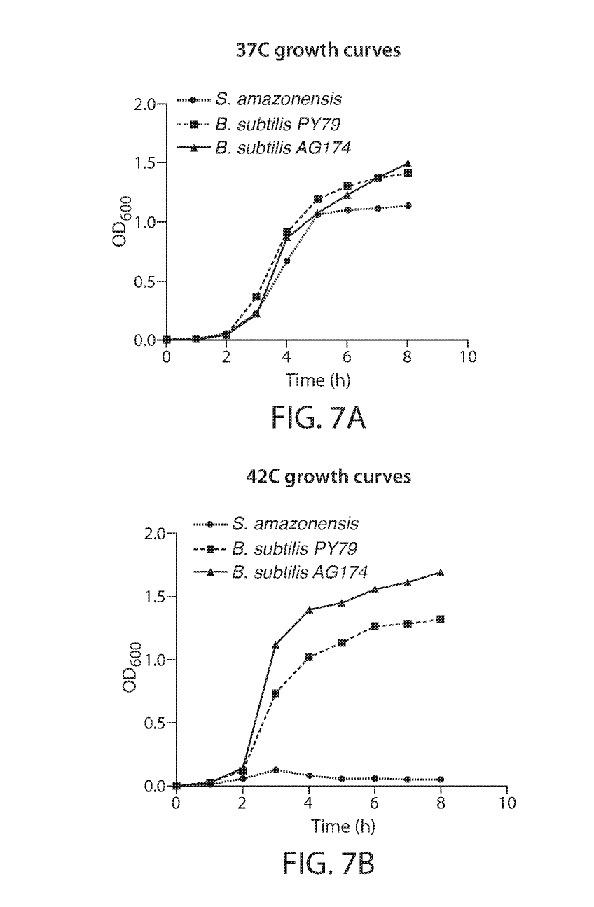

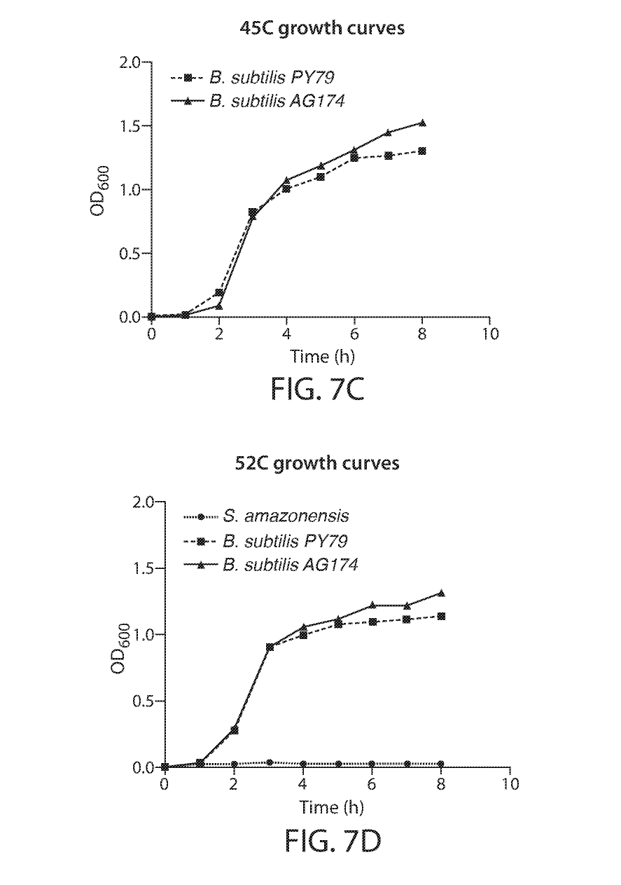

The lyophilized bacteria were then placed in sterile culture media as described above and grown at one of four different temperatures (37° C., 42° C., 45° C., 52° C.) for a total time period of 8 hours. During the culture growth, the optical density (OD at 600 nm) was measured every hour, and the results are shown in FIGS. 7A-7D.

The results shown in FIGS. 7A-7D indicate that certain strains of bacteria that have undergone sporulation, lyophilization, and reconstitution, are viable at temperatures exceeding 35° C. FIG. 7A indicates that the two strains of B. subtilis perform slightly better than S. amazonensis at 37° C. At 42° C. and 52° C., the two strains of B. subtilis are viable and undergo cell division, whereas S. amazonensis did not show marked growth, as shown in FIGS. 7B and 7D. FIG. 7C compares the growth rates of the two strains of B. subtilis at 45° C., with B. subtilis AG174 having a slightly higher optical density than B. subtilis PY79. At all the temperatures tested, B. subtilis AG174 performed slightly better than B. subtilis PY79. Since biosensors may be deposited and activated in environments where temperatures exceed 35° C., bacteria capable of surviving these temperatures, such as B. subtilis, may be a useful option for the disclosed biosensors.

### Example 2: Culture Media Comparison with Desert Temperature Simulation

Temperatures in a desert environment can range in a single day from a low of about 10° C. to a high temperature of about 45° C. Spores of B. subtilis PY79 were exposed to temperatures simulating this desert environment for a time period of 22 hours. The spores were then germinated to return the spores to their vegetative state. Germination was performed by exposing the spores to a culture medium according to known methods (J. H. Miller, “Experiments in Molecular Genetics,” Cold Spring Harbor Laboratory, (1972), pg. 433). For germination, 1 mM of L-Alanine was added to the culture media. After germination, the vegetative cells were then grown for 18 hours at 37° C. in culture media with different additives. The culture media used in this experiment are listed below in Table 1. Water was used as one additive, an amino acid (isoleucine) was added as another type of additive, and an osmolyte (glycine betaine) was added as another type of additive.

TABLE 1
Culture Media

| | Culture Media |
|---|---|
| 1 | Standard nutrient media + water |
| 2 | Standard nutrient media |
| 3 | Standard nutrient media + 100 μM isoleucine |
| 4 | Standard nutrient media + 1 mM glycine betaine |

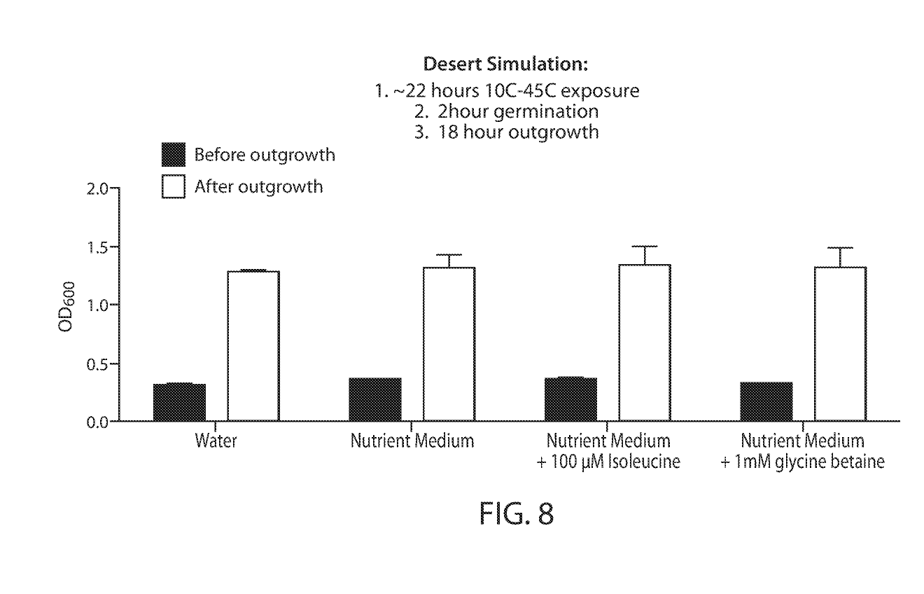

The optical density (OD at 600 nm) was measured both before outgrowth commenced, and after 18 hours, with the results shown in FIG. 8. The pre-outgrowth rates levels were all of about equivalent values. Of the different media tested, all allowed for growth. The data also indicates that certain strains of B. subtilis remain viable and capable of growth after being exposed to desert-type temperatures in their lyophilized state, and are able to grow at 37° C. once they are reconstituted.

### Example 3—Thermal Stability of DNAzyme

A lyophilized and engineered DNAzyme described in Liu et al “A catalytic beacon sensor for uranium with parts-per-trillion sensitivity and millionfold selectivity” PNAS 104:7 2056-2061 (2007) was purchased and suspended in reaction media. The DNAzyme was configured to fluoresce in the presence of the uranyl ion (UO₂²⁺), which is indicative of uranium. A first 500 nM sample of the DNAzyme was exposed to a source of uranyl ion (UO₂²⁺) at a concentration of 2 μM, and a second 500 nM sample of the DNAzyme was suspended in water without any presence of uranyl ion. A third sample was prepared that contained water and 2 μM of the uranyl analyte. One set of the three samples was kept at a temperature of 37° C., and a second set of the three samples was kept at a temperature of 52° C. Both sets were kept at their respective temperatures for a time period of 14 days.

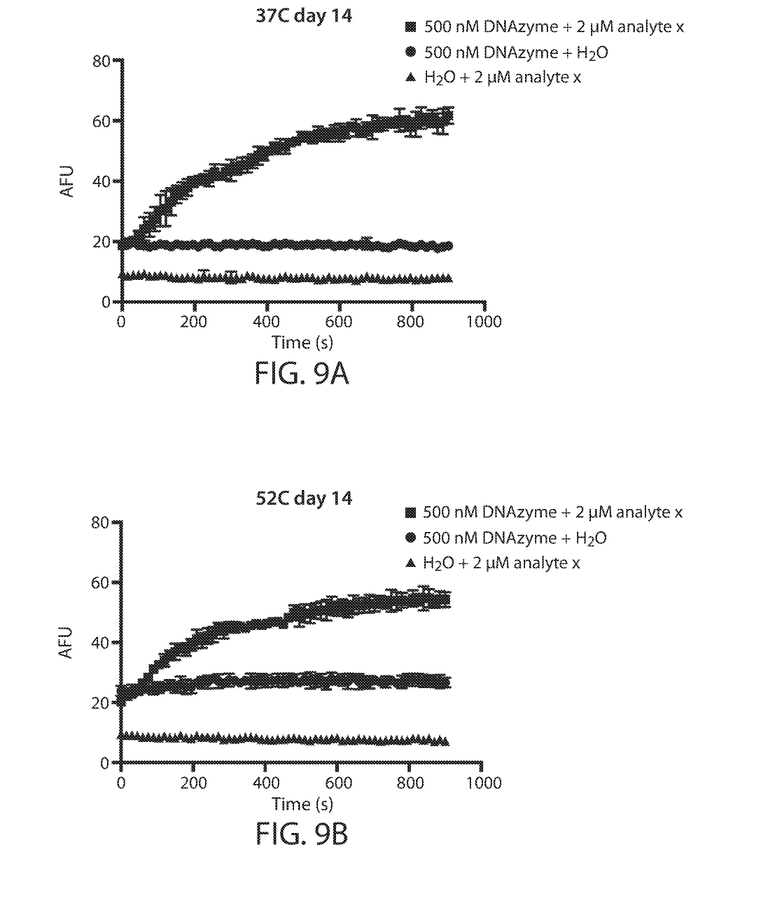

On the 14^{th} day, fluorescence measurements were taken by a fluorometer for both sets of samples. FIG. 9A plots the fluorescence (arbitrary fluorescence units, AFU) vs. time plots for the first set of samples for the 37° C. temperature condition, and FIG. 9B plots the same data for the 52° C. temperature condition. The results indicated that the engineered DNAzyme remained stable and capable of activity after reconstitution, was capable of withstanding temperatures of 52° C. for extended periods of time. In some environments, such as the desert, the DNAzyme may be exposed to temperatures reaching 52° C. may for time periods of about 6 hours per day. It is estimated that the DNAzyme is capable of remaining stable for a time period of about 56 days under these conditions.

### Example 4—Luminescent and Absorbance Data for Engineered Bacteria

B. subtilis bacteria were engineered with a chromosome-integrated transgene to detect and respond to fluoride ions by exhibiting absorption and luminescent optical characteristics. More specifically, the bacteria sense fluoride using a riboswitch according to SEQ ID NO: 1, wherein fluoride binding by the riboswitch upregulates translation of a reporter gene. For absorbance, the lacZ reporter gene was used, which can cleave a chromogenic substrate, X-gal, converting it into a blue pigment with an absorbance maxima of 615 nm, thereby turning a yellow culture green). For luminescence, the lux operon was used as a reporter.

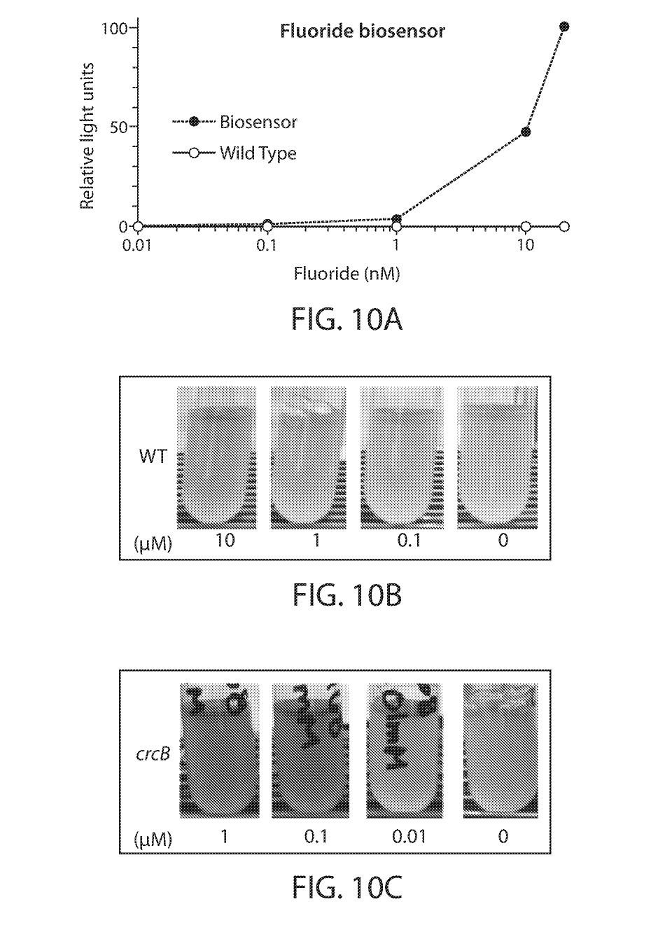

FIG. 10A is a graph comparing luminescent data for a wild type B. subtilis bacteria that was not engineered to luminesce against an engineered B. subtilis bacteria (labeled as “Luminescence Biosensor” in FIG. 10A) that were both exposed to four different concentrations of fluoride (0.0 mM, 0.1 mM, 1 mM, and 10 mM). The results indicated that the wild type exhibited no luminescent response to fluoride, whereas the engineered bacteria showed a dose-dependent increase in luminescence in response to increasing concentration of fluoride ions.

To increase sensitivity, the crcB gene of the engineered B. subtilis bacteria (specifically B. subtilis 168) was disrupted. This mutation increased the sensitivity of the system by a factor of at least 10×. FIG. 10B is a series of photographs of B. subtilis bacteria expressing a lacZ reporter as described above, and a corresponding strain where crcB is disrupted. The bacteria were exposed to four different concentration levels of fluoride (0 μM, 0.1 μM, 1 μM, and 10 μM), and the results are shown in FIG. 10B. This strain displayed stronger absorbance as the fluoride concentration increased. FIG. 10C is a series of photographs of the crcB B. subtilis bacteria at four different concentration levels of fluoride (0 μM, 0.01 μM, 0.1 μM, and 1 μM). Again, as the concentration of fluoride increased, the amount of light absorbed by the crcB B. subtilis bacteria changed (i.e., the culture displayed a color change, becoming a more intense green color). The sensitivity was much stronger for the crcB B. subtilis bacteria. FIG. 10B indicates sensitivity at a level of about 190 ppb, but other results (not shown) indicated that the crcB B. subtilis PY79 bacteria observed color change at concentrations as low as 0.001 mM (19 ppb).

The methods and systems described herein are not limited in their application to the details of construction and the arrangement of components set forth in the previous description or illustrations in the figures. The methods and systems described herein are capable of other embodiments and of being practiced or of being carried out in various ways. Also, the phraseology and terminology used herein is for the purpose of description and should not be regarded as limiting. The use of “including,” “comprising,” “having,” “containing,” “involving,” “characterized by,” “characterized in that,” and variations thereof herein is meant to encompass the items listed thereafter, equivalents thereof, as well as alternate embodiments consisting of the items listed thereafter exclusively.

Use of ordinal terms such as “first,” “second,” “third,” and the like in the specification and claims to modify an element does not by itself connate any priority, precedence, or order of one element over another or the temporal order in which acts of a method are performed, but are used merely as labels to distinguish one element having a certain name from another element having a same name, but for use of the ordinal term, to distinguish the elements.

Those skilled in the art would readily appreciate that the various parameters and configurations described herein are meant to be exemplary and that actual parameters and configurations will depend upon the specific application for which the apparatus and methods of the present disclosure are used. Those skilled in the art will recognize, or be able to ascertain using no more than routine experimentation, many equivalents to the specific embodiments described herein. For example, those skilled in the art may recognize that the system, and components thereof, according to the present disclosure may further comprise a network or systems or be a component of a sensor system. It is, therefore, to be understood that the foregoing embodiments are presented by way of example only and that, within the scope of the appended claims and equivalents thereto, the disclosed systems and methods may be practiced otherwise than as specifically described. The present system and methods are directed to each individual feature, system, or method described herein. In addition, any combination of two or more such features, systems, or methods, if such features, systems, or methods are not mutually inconsistent, is included within the scope of the present disclosure. The steps of the methods disclosed herein may be performed in the order illustrated or in alternate orders and the methods may include additional or alternative acts or may be performed with one or more of the illustrated acts omitted.

Further, it is to be appreciated that various alterations, modifications, and improvements will readily occur to those skilled in the art. Such alterations, modifications, and improvements are intended to be part of the disclosure, and are intended to be within the spirit and scope of the disclosure. In other instances, an existing system may be modified to utilize or incorporate any one or more aspects of the methods and systems described herein. Accordingly, the foregoing description and figures are by way of example only. Further, the depictions in the figures do not limit the disclosures to the particularly illustrated representations.

While exemplary embodiments of the disclosure have been disclosed, many modifications, additions, and deletions may be made therein without departing from the spirit and scope of the disclosure and its equivalents, as set forth in the following claims.

What is claimed is:

1. A method for monitoring, comprising:
pressurizing a pressure chamber to apply a first pressure to an activation fluid contained in an activation chamber;
pressurizing the activation fluid to apply a second pressure to a first membrane, the second pressure sufficient to rupture the first membrane and introduce the activation fluid to a biological chamber through the ruptured first membrane;
combining in the biological chamber the activation fluid and a dried biological component contained in the biological chamber to form a reconstituted biological component; and
pressurizing the biological chamber to apply a third pressure to a second membrane, the third pressure sufficient to rupture the second membrane and causing an entry of the reconstituted biological component to an assay chamber containing a sample such that the reconstituted biological component contacts the sample.

2. The method of claim 1, further comprising:
determining a presence or an absence of at least one analyte of interest present in the sample using at least a portion of the reconstituted biological component that has contacted the sample; and
sending a signal to a receiving device responsive to determining the presence or the absence of the at least one analyte of interest.

3. The method of claim 2, further comprising measuring at least one optical characteristic of the portion of the reconstituted biological component that has contacted the sample.

4-5. (canceled)

6. The method of claim 3, wherein determining the presence or the absence of the at least one analyte of interest is based on an electric current output.

7. The method of claim 2, wherein the at least one analyte of interest includes fluoride and uranyl ions.

8. The method of claim 1, further comprising receiving an activation signal to pressurize the pressure chamber, wherein pressurizing the pressure chamber comprises generating one or more gases within the pressure chamber.

9. (canceled)

10. The method of claim 8, wherein the pressure chamber comprises an aqueous solution, an anode, and a cathode, and generating the one or more gases comprises applying an electric current between the anode and the cathode so as to cause electrolysis of the aqueous solution.

11. The method of claim 8, wherein the gas deforms an expandable membrane positioned between the pressure chamber and the activation chamber such that the activation fluid is pressurized by the deformed expandable membrane.

12-20. (canceled)

21. An integrated sensor system comprising:
at least one unit cell, the at least one unit cell including:
an activation chamber containing an activation fluid;
a biological chamber containing a dried biological component;
a first membrane disposed in between the activation chamber and the biological chamber;
an assay chamber configured to receive a sample; and
a second membrane disposed in between the assay chamber and the biological chamber;
an analysis device in communication with the assay chamber and configured to transmit an output signal based on detection of at least one characteristic of a biological component in contact with the sample; and
a controller in communication with the analysis device and configured to receive the output signal.

22. The integrated sensor system of claim 21, further comprising:
a pressure chamber; and
an expandable membrane disposed between the pressure chamber and the activation chamber and configured to deform such that the expandable membrane applies pressure to the activation fluid.

23. The integrated sensor system of claim 22, wherein the pressure chamber further includes at least one electrode.

24. The integrated sensor system of claim 23, wherein the controller is configured to send an activation signal to the pressure chamber such that a voltage is applied the at least one electrode, and the pressure chamber contains a pressurization fluid.

25-27. (canceled)

28. The integrated sensor system of claim 21, wherein biological component comprising the dried biological component is configured to detect at least one analyte and to exhibit the at least one characteristic in the presence of the analyte.

29. The integrated sensor system of claim 28, wherein the unit cell further comprises a housing that at least partially encapsulates the activation chamber and the biological chamber.

30. The integrated sensor system of claim 29, wherein the housing is constructed from a light-transmissive material.

31. The integrated sensor system of claim 30, wherein the at least one characteristic is an optical characteristic selected from the group consisting of fluorescence, luminescence, and an absorption parameter.

32. (canceled)

33. The integrated sensor system of claim 31, wherein the analysis device is a fluorimeter configured to measure fluorescence.

34. The integrated sensor system of claim 29, wherein the housing includes a patterned electrode configured to detect an electric current output by the portion of reconstituted biological component that contacted the sample and the analysis device is configured to measure the electric current.

35-37. (canceled)

38. The integrated sensor system of claim 21, wherein the activation fluid is at least one of a culture media and a reaction media.

39. The integrated sensor system of claim 21, wherein the dried biological component is at least one of an engineered biotic and an engineered abiotic.

40. The integrated sensor system of claim 39, wherein the engineered biotic comprises at least one genetically engineered microbe.

41. The integrated sensor system of claim 40, wherein the genetically engineered microbe is a bacteria.

42. The integrated sensor system of claim 39, wherein the engineered abiotic comprises at least one engineered DNA molecule.

43. (canceled)

44. The integrated sensor system of claim 21, wherein the reconstituted biological component is formed from at least a portion of the activation fluid entering through the first membrane and reconstituting at least a portion of the dried biological component contained in the biological chamber.

45-46. (canceled)

47. A method of facilitating an integrated sensor system for monitoring one or more target analytes, comprising:
providing an integrated sensor system, the integrated sensor system comprising at least one unit cell, the at least one unit cell including:
an activation chamber containing an activation fluid;
a biological chamber containing a dried biological component;
a first membrane disposed in between the activation chamber and the biological chamber;
an assay chamber configured to receive a sample; and
a second membrane disposed in between the assay chamber and the biological chamber;
an analysis device in communication with the assay chamber and configured to transmit an output signal based on detection of at least one characteristic of a biological component in contact with the sample; and
a controller in communication with the analysis device and configured to receive the output signal; and
providing instructions for activating the integrated sensor system.
